(19) United States
(12) Patent Application Publication
(10) Pub. No.: US 2020/0375868 A1
(43) Pub. Date: Dec. 3, 2020

(54) Systemic Delivery of Polypeptides

(71) Applicants: President and Fellows of Harvard College, Cambridge, MA (US); Massachusetts Institute of Technology, Cambridge, MA (US); The Brigham and Women's Hospital, Inc., Boston, MA (US); The General Hospital Corporation, Boston, MA (US)

(72) Inventors: Denitsa M. Milanova, Boston, MA (US); George M. Church, Brookline, MA (US); Noah Davidsohn, Brookline, MA (US); Carl Schoellhammer, Medford, MA (US); Robert S. Langer, Newton, MA (US); Anna I. Mandinova, Newton, MA (US); Carlo Giovanni Traverso, Newton, MA (US); Li Li, Cambridge, MA (US)

(21) Appl. No.: 16/612,632

(22) PCT Filed: May 14, 2018
(86) PCT No.: PCT/US18/32597

§ 371 Date: Nov. 11, 2019

Related U.S. Application Data
(60) Provisional application No. 62/505,359, filed on May 12, 2017.

(51) Int. Cl. A61K 8/64; C12N 15/86; A61K 8/99; A61Q 19/08

(52) U.S. Cl. CPC A61K 8/64; C12N 15/86; A61K 8/99; C12N 2750/14143; A61K 2800/82; A61K 2800/86; A61Q 19/08

(57) ABSTRACT

A method of delivering a recombinant virus to a skin tissue is provided. The method includes applying ultrasound to the skin tissue, and administering the recombinant virus to the skin tissue.

### RELATED APPLICATION DATA

This application is a National Stage Application under 35 U.S.C. 371 of co-pending PCT application PCT/US18/32597 designating the United States and filed May 14, 2018; which claims the benefit of U.S. provisional application No. 62/505,359 filed on May 12, 2017 each of which are hereby incorporated by reference in their entireties.

### STATEMENT OF GOVERNMENT INTERESTS

This invention was made with government support under Grant Nos. HG008525, MH113279, and EB000244 from the National Institutes of Health. The government has certain rights in the invention.

### BACKGROUND

Gene therapy has shown great promise to prevent, treat and cure a variety of skin diseases and conditions in human and animals. Skin is the largest and one of the most complex organs in the human body. It performs a diverse set of functions, ranging from protection, sensation, heat regulation, absorption of gases, excretion of sweat, control of evaporation and water resistance. Skin's structure and function gradually deteriorate with age (intrinsic aging) and in response to varying environmental conditions (extrinsic aging) such as exposure to solar radiation and a variety of chemicals becoming prone to common benign and malignant skin lesions such as Seborrheic keratosis, Actinic keratosis and non-melanoma skin cancers. Furthermore, skin's health gradually declines in response to chronic conditions including HIV, diabetes, atherosclerosis, and even inadequate nutrition. As a result, skin accumulates high mutational loads evinced in altered translation of key proteins maintaining skin homeostasis. At tissue level, the stratum corneum loses its ability to barrier function, regeneration and wound healing; the epidermis becomes prone to errors in metabolic reprogramming and the rete ridges lose surface area; the dermis becomes thinner and less elastic; the sebaceous and eccrine glands contract and secrete less oils and sweat; and the Langerhans immune cells decline in number and function.

As a superficial organ, the skin is an easily accessible target for gene therapy. To perform gene therapy, recombinant viral vectors have been developed as attractive alternatives to non-viral vectors to deliver genes and nucleic acid molecules of interest to the skin. These recombinant viral vectors include recombinant retrovirus, adenovirus, adeno-associated virus (AAV), vaccinia virus and herpes simplex virus. However, the efficacy of skin gene therapy is hampered by low level of transgene expression, due to difficulty of viral permeation in the skin tissue. There is a continuing need in the art to improve the efficacy of skin gene therapy by enhancing viral permeation in the skin tissue.

### SUMMARY

Aspects of the present disclosure are directed to methods of non-invasive delivery of nucleic acid molecules including genes via recombinant viral vectors to skin tissue in vivo and in vitro. In certain embodiments, the delivery method comprises electroporation such as by applying short high voltage pulses to the skin, heat (between about 32° C.-39° C.), needleless injections such as by firing liquid at supersonic speed through the stratum corneum, pressure waves generated by laser radiation, fraction laser, or radiofrequency (about 100 kHz), magnetophoresis by external magnetic field, iontophoresis, chemical peels, abrasion techniques such as diamond or sand paper abrasion, tape stripping, and the like. In an exemplary embodiment, ultrasonic pre-treatment of skin tissue is used to enable increased tissue permeation before administering the recombinant viruses to the treated skin tissue. In one embodiment, recombinant adeno-associated viruses are used to deliver nucleic acid molecules of interest to skin tissue/cells to modify target gene expression. The methods disclosed herein are suitable for simultaneously modifying the expression of sets of target genes involved in maintaining skin homeostasis and health. Aspects of the present disclosure are directed to methods of introducing nucleic acid molecules comprising nucleic acid sequences for expression in skin cells. The nucleic acid sequences encode RNA and polypeptides that function to activate or repress target gene expression. The nucleic acid sequences can also integrate into the cell's genome and modulate target gene expression. Recombinant viral vectors are employed to package and deliver the nucleic acid molecules. For example, nucleic acid molecules are packaged in recombinant adeno-associated viral (rAAV) vectors. The methods of the present disclosure have demonstrated long-term transgene expression and modulated protein translation from rAAV vectors in animal (in vivo) and human (ex vivo and in vitro) experimental models. In some embodiments, the methods of the present disclosure include optimal tissue specificity and efficiency of gene transfer based on rAAV vector serotypes such that these vectors selectively target one, more, or all skin tissue layers and structures (i.e. stratum corneum, epidermis, basement membrane, dermis, hair follicles, and sebaceous and eccrine glands). The methods of the present disclosure improve skin gene therapies and are well-suited to enable reversal of skin aging phenotypes and phenotypes resulting from complex disease- and age-associated skin pathologies.

According to one aspect, a method of delivering a recombinant virus to a skin tissue is provided. In one embodiment, the method includes applying ultrasound to the skin tissue, and administering the recombinant virus to the skin tissue. In one embodiment, the recombinant virus is delivered to the skin tissue of a subject in vivo. In some embodiments, the skin tissue comprises native and autogeneic, isogeneic, xenogeneic and allogeneic skin tissue. In another embodiment, the recombinant virus is delivered to the skin tissue in vitro. In some embodiments, the skin tissue comprises skin explants and artificial skin tissues. In one embodiment, the ultrasound is applied prior to administering the recombinant virus. In another embodiment, the ultrasound is stopped prior to administering the recombinant virus. In some embodiments, the ultrasound is applied at a frequency between about 20 kHz and about 100 kHz. In other embodiments, the ultrasound is applied at a frequency of about 10 kHz, 20 kHz, 30 kHz, 40 kHz, 50 kHz, 60 kHz, 70 kHz, 80 kHz, 90 kHz and 100 kHz. In some embodiments, the ultrasound is applied at an intensity of about 1, 2, 3, 4, 5, 6, 7, 8, 9 and 10 W/cm². In other embodiments, the ultrasound is applied at an intensity between about 1 W/cm² and about 10 W/cm². In some embodiments, the ultrasound is applied for a duration between about one minute to about 10 minutes. In other embodiments, the ultrasound is applied for a duration of about 1, 2, 3, 4, 5, 6, 7, 8, 9 and 10 minutes. In some embodiments, the ultrasound is applied at duty cycles in the range between about 10% and 100%. In some embodiments, the ultrasound is applied at duty cycles in the range between about 10%, 25%, 50%, 75% and 100%. In certain embodiments, the ultrasound is applied topically or intra-dermally. In other embodiments, the methods further include delivering the recombinant virus to the skin tissue via electroporation, heat, needleless injections, pressure waves generated by laser radiation, fraction laser, or radiofrequency (100 kHz), magnetophoresis by external magnetic field, iontophoresis, chemical peels, abrasion techniques including diamond or sand paper abrasion, tape stripping, and the like. In some embodiments, the recombinant virus is selected from retrovirus, adenovirus, adeno-associated virus (AAV), vaccinia virus and herpes simplex virus. In other embodiments, the recombinant AAV includes serotypes 1-9. In one embodiment, the recombinant virus comprises a heterologous nucleic acid sequence. In another embodiment, the nucleic acid sequence encodes a gene which is expressible in the skin tissue. In one embodiment, expression of the gene effects treatment of a skin disease or condition. In certain embodiments, the gene is selected from COL1A1, COL3A1, TIMP1, TIMP2, SMAD2, SMAD3, CTGF, TGF-beta1, KRT6A, NOTCH1(icd), TET2, TET3, Sirt1, Sirt6, Pck1, Pparg, and Cisd2, MDH1, MDH2, Aco1, Aco2, IDH1, IDH2, IDH3, ENO1, GOT1, GOT2, MUC1, and MCU. In one embodiment, the gene encodes a green fluorescent protein (GFP). In some embodiments, the skin disease or condition includes Epidermolysis Bullosa, Recessive Dystrophic Epidermolysis Bullosa, Junctional Epidermolysis Bullosa, Epidermolysis Bullosa Simplex, Pachyonychia Congenita, Melanoma, non-melanoma skin cancer, Ichthyosis, Harlequin Ichthyosis, Sjogren-Larsson Syndrome, Xeroderma Pigmentosum, Wound Healing, Netherton Syndrome, age-associated skin pathologies, benign and malignant skin lesions, inflammatory and autoimmune skin disorders. In other embodiments, the recombinant virus is delivered to keratinocytes, epidermal stem cells, fibroblast cells, mesenchymal stem cells, immune cells, melanocytes, vascular endothelial cells, adipocytes, Merkel cells and peripheral neural cells of the skin tissue. In some embodiments, the recombinant virus is delivered to skin tissue layers and structures including stratum corneum, epidermis, basement membrane, dermis, hair follicles, blood vessels and sebaceous and eccrine glands. In certain embodiments, multiple recombinant viruses comprising multiple genes are delivered to the skin tissue. In some embodiments, the subject is human or non-human mammal. In other embodiment, the non-human mammal is selected from a mouse, rat, cow, pig, sheep, goat, and horse.

According to another aspect, a recombinant virus comprising a heterologous nucleic acid sequence is provided. In one embodiment, the nucleic acid sequence encodes a gene which is expressible in a skin tissue. In some embodiments, expression of the gene effects treatment of a skin disease or condition. In certain embodiments, the gene is selected from COL1A1, COL3A1, TIMP1, TIMP2, SMAD2, SMAD3, CTGF, TGF-beta1, KRT6A, NOTCH1(icd), TET2, TET3, Sirt1, Sirt6, Pck1, Pparg, and Cisd2, MDH1, MDH2, Aco1, Aco2, IDH1, IDH2, IDH3, ENO1, GOT1, GOT2, MUC1, and MCU. In one embodiment, the gene encodes a green fluorescent protein (GFP). In some embodiments, the recombinant virus is selected from retrovirus, adenovirus, adeno-associated virus (AAV), vaccinia virus and herpes simplex virus. In other embodiments, the recombinant AAV includes serotypes 1-9.

According to yet another aspect, a method of delivering a polypeptide to a skin tissue is provided. In one embodiment, the method includes applying ultrasound to the skin tissue, and administering a nucleic acid sequence encoding the polypeptide to the skin tissue. In another embodiment, the nucleic acid sequence encoding the polypeptide is delivered to the skin tissue of a subject in vivo. In one embodiment, the skin tissue comprises native and autogeneic, isogeneic, xenogeneic and allogeneic skin tissue. In another embodiment, the nucleic acid sequence encoding the polypeptide is delivered to the skin tissue in vitro. In one embodiment, the skin tissue comprises skin explants and artificial skin tissues. In another embodiment, the ultrasound is applied prior to administering the nucleic acid sequence encoding the polypeptide. In one embodiment, the ultrasound is stopped prior to administering the nucleic acid sequence encoding the polypeptide. In some embodiments, the ultrasound is applied at a frequency between about 20 kHz and about 100 kHz. In other embodiments, the ultrasound is applied at a frequency of about 10 kHz, 20 kHz, 30 kHz, 40 kHz, 50 kHz, 60 kHz, 70 kHz, 80 kHz, 90 kHz and 100 kHz. In some embodiments, the ultrasound is applied at an intensity of about 1, 2, 3, 4, 5, 6, 7, 8, 9 and 10 W/cm². In other embodiments, the ultrasound is applied at an intensity between about 1 W/cm² and about 10 W/cm². In some embodiments, the ultrasound is applied for a duration between about one minute to about 10 minutes. In other embodiments, the ultrasound is applied for a duration of about 1, 2, 3, 4, 5, 6, 7, 8, 9 and 10 minutes. In some embodiments, the ultrasound is applied at duty cycles in the range between about 10% and 100%. In some embodiments, the ultrasound is applied at duty cycles in the range between about 10%, 25%, 50%, 75% and 100%. In certain embodiments, the ultrasound is applied topically or intra-dermally. In certain embodiments, the nucleic acid sequence encoding the polypeptide is DNA or RNA. In one embodiment, wherein the polypeptide is expressible in the skin tissue. In another embodiment, expression of the polypeptide effects treatment of a skin disease or condition. In some embodiments, the nucleic acid sequence encodes a gene selected from COL1A1, COL3A1, TIMP1, TIMP2, SMAD2, SMAD3, CTGF, TGF-beta1, Anti-MMP1, anti-MMP2, KRT6A, NOTCH1(icd), TET2, TET3, Sirt1, Sirt6, HIF-1a, Pten, Pck1, Pparg, and Cisd2, MDH1/2, Aco1/2, IDH1/2/3, Enolase, GOT1/2, MUC1, and MCU. In one embodiment, the nucleic acid sequence encodes a green fluorescent protein (GFP). In some embodiments, the skin disease or condition includes Epidermolysis Bullosa, Recessive Dystrophic Epidermolysis Bullosa, Junctional Epidermolysis Bullosa, Epidermolysis Bullosa Simplex, Pachyonychia Congenita, Melanoma, non-melanoma skin cancer, Ichthyosis, Harlequin Ichthyosis, Sjogren-Larsson Syndrome, Xeroderma Pigmentosum, Wound Healing, Netherton Syndrome, age-associated skin pathologies, benign and malignant skin lesions, inflammatory and autoimmune skin disorders. In some embodiments, the nucleic acid sequence encoding the polypeptide is delivered to keratinocytes, epidermal stem cells, fibroblast cells, mesenchymal stem cells, immune cells, melanocytes, vascular endothelial cells, adipocytes, Merkel cells and peripheral neural cells of the skin tissue. In other embodiments, the nucleic acid sequence encoding the polypeptide is delivered to skin tissue layers and structures including stratum corneum, epidermis, basement membrane, dermis, hair follicles, blood vessels and sebaceous and eccrine glands. In some embodiments, multiple nucleic acid sequences encoding multiple polypeptides are delivered to the skin tissue. In some embodiments, native polypeptide is delivered to the skin tissue.

According to one aspect, a heterologous nucleic acid sequence encoding a gene which is expressible in a skin tissue is provided. In one embodiment, expression of the gene effects treatment of a skin disease or condition. In some embodiments, the heterologous nucleic acid encodes a gene selected from COL1A1, COL3A1, TIMP1, TIMP2, SMAD2, SMAD3, CTGF, TGF-beta1, Anti-MMP1, anti-MMP2, KRT6A, NOTCH1(icd), TET2, TET3, Sirt1, Sirt6, HIF-1a, Pten, Pck1, Pparg, Cisd2, MDH1/2, Aco1/2, IDH1/2/3, Enolase, GOT1/2, MUC1, and MCU. In one embodiment, the heterologous nucleic acid sequence encodes a green fluorescent protein (GFP).

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 shows images and image processing algorithm for the estimation of native EGFP fluorescence in large skin tissue sections.

FIGS. 2 A & C show results of fold increase in signal intensity relative to the signal of negative control which was treated with ultrasound but no therapy was administered. FIGS. 2 B & D show results of percent transduced tissue area in 30-year old (A, B) and 52-year-old (C, D) donors.

FIGS. 3A-3E show immunofluorescent images of human breast skin explants treated with recombinant AAV vectors expressing EGFP. The tissues were stained with Vimentin, anti-EGFP, and Cytokeratin 19. FIG. 3A. Composite overlay images in 4 fluorescent channels, starting from blue, green, red, and far red. FIG. 3B. anti-EGFP images for control+treated with AAV2/1 AAV2/2, AAV2/8, and AAV2/9. Higher magnification images for exemplary skin structures: FIG. 3C. Epidermis (AAV2/2), FIG. 3D. Hair follicle and the niche (AAV2/1), and FIG. 3E. Sebaceous glands (AAV2/9). All rAAV hybrids are packaged with EGFP and used to quantify gene transfer efficiency.

FIGS. 4A-4D depict immunofluorescent images of human facial skin explants treated with recombinant AAV vectors expressing EGFP.-control (FIG. 4A and FIG. 4B) vs treated with AAV2/2: EGFP (signal is shown in white) (FIG. 4C and FIG. 4D). FIG. 4A. K15-positive, EGFP-negative proliferating stem cells in hair follicles. FIG. 4B. K15-positive, EGFP-negative stem cells located in the basement membrane. FIG. 4C. K15-positive, EGFP-positive proliferating stem cells in hair follicles. FIG. 4D. K15-positive, EGFP-positive stem cells located in the basement membrane.

FIGS. 5A-5F show methods and results of in vivo delivery of gene therapy. FIGS. 5A-5D (Steps 1-4). FIG. 5A. Step 1: Skin was pretreated via ultrasound of 5 W/cm² intensity, 50% duty cycle of 30s, and 20 kHz frequency; FIG. 5B. Step 2: rAAV2/2: COL3A1 was delivered topically; FIG. 5C. Step 3: therapy was let passively diffuse; and FIG. 5D. Step 4: tissue was harvested and analyzed by Western blot to quantify protein content. FIG. 5E Western blot and FIG. 5F protein quantification of Western blot for target gene COL3A1 and housekeeping gene ACTB in human skin control samples, and for two biological replicates (2 hairless mice). The negative control tissue was taken from the mouse stomach. Signal is normalized relative to the protein expression of ACTB and negative untreated control tissue in two biological replicates. Human skin was used as a control tissue for Collagen III amount. Error bars represent the standard error to the mean of 3 tissue samples within a biological replicate. Native content of Collagen III in human skin is shown as positive control.

FIGS. 6A-6G show methods and results of in vitro delivery of rAAV2/2: COL3A1. FIGS. 6A-6D. (Steps 1-4). FIG. 6A. Step 1: Skin was pretreated via ultrasound of 5 W/cm² intensity, 50% duty cycle of 30s, and 20 kHz frequency; FIG. 6B. Step 2: rAAV2/2: COL3A1 was delivered topically; FIG. 6C. Step 3: therapy was let passively diffuse; and FIG. 6D. Step 4: tissue was harvested and analyzed by RT-qPCR and Western blot to quantify changes in gene expression protein content, respectively. FIG. 6E. RT-qPCR of GFP and COL3A1 expression in samples treated with rAAV2/2: EGFP and rAAV2/2: COL3A1. Signal is expressed as fold change overexpression of target genes (GFP and COL3A1) normalized to ACTB expression and negative untreated control sample; FIG. 6F. Protein quantification using Western blot for target genes GFP and COL3A1 and housekeeping gene ACTB. Signal is normalized relative to the protein expression of ACTB and negative untreated control tissue in a single reconstructed skin tissue. Untreated tissue was used as a control, and FIG. 6G. Histological analysis of control and rAAV2/2: COL3A1-treated tissue samples using Picro-sirius red and trichrome staining (arrows point to regions of newly synthesized collagen fibrils).

FIGS. 7A-7B show schematic illustration of the network propagation method according to an embodiment of the disclosure. FIG. 7A shows three identical networks before network propagation with three different nodes were assigned with values. FIG. 7B demonstrates the network after propagation. Higher brightness of a node responds to a higher score.

FIGS. 8A-8B show the visualization results of the networks according to certain embodiments of the disclosure. FIG. 8A shows a proposed network built upon the top 10 most significantly enriched (non-disease) KEGG pathways of the analysis with an FDR q-value<0.01. FIG. 8B shows the top genes with highest scoring (as generated by integrating the distant network and gene expression of the neighborhood) involved in one or multiple of the enriched top pathways.

FIGS. 9A-9B show results of MDH2 levels in aging skin progenitors of primary cultures. FIG. 9A shows protein production of MDH2 in aging skin progenitors of primary cultures that were measured using Western blot. FIG. 9B shows quatitative measure of FIG. 9A in bar graph.

FIGS. 10A-10H show results of gene transfer to whole skin according to certain embodiments of the disclosure. FIG. 10A shows a schematic of a length-optimized modular vector with EGFP gene inserted. FIG. 10B shows a typical workflow of topical delivery of AAV vectors to human skin explants pre-treated with low frequency (20 kHz) ultrasound. FIG. 10C shows results of EGFP expression levels in human skin explants after AAV-treatment, human skin explants were cultured in 1 cm-transwells for 8 days after which tissues were analyzed for gene expression. FIG. 10D shows the results of the absolute gene expression copy number that was evaluated based on a standard curve built upon known amounts of input transgene. FIGS. 10C and 10D show mean and standard error to the mean of N=2 replicates. FIGS. 10E-10F show results of AAV2 EGFP expression levels administered to human skin explants under various promoters. FIGS. 10G-10H show results of AAV2/8-hEF1a-EGFP expression levels administered to human skin explants.

FIGS. 11A-11E show results of gene delivery efficiency to dermal skin cells according to certain embodiments of the disclosure. FIG. 11A shows the process for one untreated, one ultrasound-treated, and one AAV-treated tissue sample. A schematic illustration of AAV-CMV-EGFP vector is shown in FIG. 11B. Recombinant AAV viruses of serotypes 2/1, 2/2, 2/5, 2/6.2, 2/8, 2/9 were administered at a dose of 2E+11 GC per tissue explant and the fluorescence signal is reported for two donors, one young (of ages 30) and one old (of age 52) as shown in FIG. 11C. FIG. 11D shows a heatmap illustrating the amount of protein expression in the tissue samples. FIG. 11E shows the results of EGFP expression in populations of single EGFP-positive cells and double EGFP/K15-positive cells.

FIGS. 12A-12C show the results of EGFP expression in keratinocyte cells. FIG. 12A shows EGFP levels in various AAV serotypes. FIG. 12B shows EGFP levels using AAV2/2 at a dose of 2E+11 GC per explant under CMV, CASI, shEF1a, and hEF1a promoters. FIG. 12C shows a dose dependency response using AA8-hEF1a from 5E+10 to 5E+11 GC per explant.

FIGS. 13A-13D show results of long-term expression of genes in skin tissues according to certain embodiments of the disclosure. FIG. 13A shows the differentiated keratinocyte population that was further analyzed for therapy efficacy towards progenitor stem cells expressing markers either for Cytokeratin 15, a6-Integrin, or both. Based on their ability to infect progenitor and stems cells, the top 5 most efficacious AAV-serotypes measured by GFP and K15 signal are listed in FIG. 13B. FIG. 13C shows results of expression of K15 and a6-integrin using various AAV vectors. FIG. 13D shows the results of the correlation of infection towards epidermal stem and progenitor cells.

FIGS. 14A-14E show results of expression of human collagen III (alpha domain) driven by a truncated hEF1a promoter in human skin explants according to certaine embodiments of the disclosure. FIG. 14A shows a diagram of delivery of rAAV to skin using low frequency ultrasound. FIG. 14B shows results of Collagen III expression in the skin explants. FIG. 14C shows the results of protein levels for Collagen III analyzed by Western blot. FIG. 14D shows results of Collagen III expression in the another donor skin explants. FIG. 14E shows the results of protein levels for Collagen III analyzed by Western blot.

FIGS. 15A-15B shows the results of modulation of 4 age-related genes in SKH-1E hairless mice. FIG. 15A shows (mouse) KRT6A, (human) TET3, (mouse) TGFb1, and (human) COL3A1 genes. FIG. 15B shows results of gene modulation of the four age-associated genes in SKH-1E hairless mice.

FIGS. 16A-16C show results of long-term expression of Collagen III in in vivo skin rebuilding of skin's extracellular matrix according to certain embodiments of the disclosure. FIG. 16A shows a collagen III production curve as a function of time from 1 week to 32 weeks. FIG. 16B show expression protein levels that were determined by Western blot on mouse skin lysates for N=8 mice. FIG. 16C shows collagen III levels in human skin and levels were compared relative to the last data point in the mouse in vivo experiment.

FIGS. 17A-17B show the results of ultraclean production and purification of rAAV according to certain embodiments of the disclosure. FIG. 17A shows results of high VP protein purity. FIG. 17B shows an image of the virus under the transmission electron microscopy.

FIGS. 18A-18B show an inflammatory panel at Day 3 and Day 8, respectively, run on epidermal cells dissociated from human skin explants after treatment with rAAV-GFP therapy via ultrasound. At Day 3 (FIG. 18A), no inflammatory response above the baseline levels was detected, while at Day 8 (FIG. 18B) a minor transient response was observed as evidenced by slightly increased gene expression levels of Interferon alpha-1 (INFa1) and Interferon beta-1 (INFb1).

### DETAILED DESCRIPTION

The present disclosure describes a method of systemic delivery of a polypeptide to a subject including genetically modifying target skin cells within skin of a subject using an engineered virus or nucleic acid sequences. The engineered virus includes one or more genomic nucleic acid sequences and one or more foreign nucleic acid sequences encoding one or more target polypeptides. The one or more genomic nucleic acid sequences and the one or more nucleic acid sequences encoding one or more target polypeptides are introduced into the target skin cells to produce genetically modified target skin cells. The genetically modified target skin cells produce the one or more target polypeptides.

According to one aspect, an engineered virus is administered to the skin of the subject in a manner to direct the engineered virus to the target skin cells. Various administration methods are contemplated including electroporation, heat, needleless injections, pressure waves generated by laser radiation, fraction laser, or radiofrequency (about 100 kHz), magnetophoresis by external magnetic field, iontophoresis, chemical peels, abrasion techniques including diamond or sand paper abrasion, tape stripping, and the like.

According to one aspect, the skin of the subject may be treated so as to permeabilize the stratum corneum to the presence of the engineered virus or nucleic acid sequences or otherwise improve efficiency of the engineered virus or nucleic acid sequences to traverse the stratum corneum to the target skin cells. After treating the skin surface, the engineered virus or nucleic acid sequences may be topically administered to the skin surface and the engineered virus or nucleic acid sequences may passively diffuse to the target skin cells whereupon the engineered virus infects the target cells to include the one or more nucleic acid sequences encoding one or more target polypeptides, or whereupon the nucleic acid sequences encoding one or more target polypeptides transduce the target cells. The one or more target polypeptides are produced by the genetically modified target cells. In some embodiments, the one or more target polypeptides are excreted from the genetically modified target cells and into the blood stream of the subject. According to one aspect, the one or more target polypeptides are excreted from the genetically modified target cells in a manner to provide a prolonged release of the one or more target polypeptides into the bloodstream of the subject.

According to one aspect, a delivery platform is provided that utilizes human skin to enable a single-step, extended production, such as year-long production of biologics wherein gene-encoded vectors are topically administered to skin in a non-invasive manner so as to treat or prevent a disease. Skin cells are provided with non-integrative viral vectors which, according to one embodiment, may lack specific cytotoxicity and pathogenicity. Delivery of the viral vectors is achieved by “needleless” methods leveraging breakage of the stratum corneum. The genetic modification of skin cells to include the gene-encoded vectors provides for long-lived and efficient translation of a polypeptide, such as a therapeutic agent in vivo to provide a safe and effective gene transfer for treatment or prevention. According to one aspect, skin is pretreated using noninvasive technology, such as ultrasound or microdermabrasion, to premeabilize or score or remove the stratum corneum. The engineered virus, such as a gene-encoding adeno-associated virus (“AAV particles”) is topically administered and delivered to the pretreated skin, which may be a section of skin near active lymph nodes. According to one aspect, target cells, such as dermal fibroblasts, endosome the AAV particles and the AAV particles release the DNA contained therein into the fibroblast cell nucleus. The fibroblast cells translate and secrete the one or more polypeptides to the intercellular matrix of the skin tissue or blood stream. The polypeptides are present within the intercellular matrix of the skin tissue or blood system for therapy or prevention. For example, the one or more polypeptides may be broadly neutralizing antibodies present within the intercellular matrix of the skin tissue or the blood system to prevent infection. In this manner, the skin may be transformed into an in vivo bioreactor for the production of biologics, such as antibodies, for transfer into the blood stream.

Embodiments of the present disclosure are directed to methods of delivering nucleic acid molecules of interest via recombinant viruses to a skin tissue. In addition to gene therapies correcting for one gene as in rare genetic diseases, the disclosed method also includes delivery of multiple sets of genes along key aging and disease signaling pathways affecting skin tissues so as to globally restore healthy and youthful transcriptional and translational profiles of skin cells and tissues. In exemplary embodiments, the method includes two major steps. In step one, ultrasound is applied to a skin tissue to increase tissue permeation. In step two, recombinant viruses carrying foreign nucleic acid molecule(s)/gene(s) of interests are delivered to the skin tissue.

Ultrasound treatment of skin has been known. A skilled in the art can choose the appropriate ultrasound device according to an application. To increase skin tissue permeation, ultrasound is applied to the skin tissue. A skilled in the art can determine the frequency, intensity and duration of ultrasound application that is effective for a specific purpose. In an exemplary embodiment, a treatment with ultrasound at 20 kHz frequency is applied at an intensity of less than 8 W/cm² for up to one minute at 50% duty cycle. The ultrasonic pre-treatment of skin tissue improves tissue diffusivity by increasing its effective diffusion coefficient. This process is enabled by the disruption of skin's stratum corneum.

Alternatively, other delivery methods can be used to deliver the recombinant viruses to the skin. These delivery methods comprise electroporation such as by applying short high voltage pulses to the skin, heat (between about 32° C. and 39° C.), needleless injections such as by firing liquid at supersonic speed through the stratum corneum, pressure waves generated by laser radiation, fraction laser, or radiofrequency (about 100 kHz), magnetophoresis by external magnetic field, iontophoresis, chemical peels, abrasion techniques such as diamond or sand paper abrasion, tape stripping, and the like. A skilled in the art can choose the appropriate delivery method according to an application. These methods can be used in combination with the method of ultrasound pre-treatment of skin and administering of the recombinant viruses as disclosed herein.

According to one aspect, viral vectors may be selected based on the ability to target cell types in a specific manner. Such viral vectors may be identified by multiplexed screening of hybrid capsid variations of adeno-associated viruses (“AAVs”). Hybrid AAV constructs typically exhibit less immunogenicity than the wild-type AAV, and have greater tissue specificity.

A large set of existing viral serotypes is optimized, synthesized and tested in human organotypic cultures. Human abdominal skin is cultured ex vivo, using native fluorescence of reporter genes, FACS, and in situ screening approaches. The method is high-throughput, allows for combinatorial optimization, and accounts for donor-to-donor variability related to immune response and metabolic state. According to one aspect, a human skin explant model is utilized that preserves the physiological complexity, the proliferative capacity and the structural integrity of all skin components for up to 28 days. Viable explants are utilized with a surface area of 15-20 mm to enable topical treatment with test agents and compositions.

According to one aspect, rAAV vector serotypes exhibit tissue specificity and efficiency of gene transfer. To establish delivery efficiency, the native fluorescence was studied of a reporter gene (rAAV: EGFP) distributed over a large surface area in full thickness human (breast) skin tissues (16 mm×2 mm in cross-sectional area) maintained in a culture dish for 24 hours, post-treatment. To enable quantification, the native fluorescence was studied of a reporter gene distributed over a large surface area in full thickness human (breast) skin tissues cultured for 24 hours post-treatment. The signal of EGFP in frozen samples (16 mm×1 mm×20 μm) was analyzed and quantified using a custom MatLab code for image post-processing. This algorithm executes flat-field and background corrections and creates a logical mask of the image. According to one aspect, the skin of the subject may be treated prior to topical application of the engineered virus so as to permeabilize the stratum corneum or otherwise The use of recombinant RNA or DNA viral based vector systems for the delivery of nucleic acids take advantage of highly evolved processes for targeting a virus to specific cells in the skin tissue and trafficking the viral payload to the nucleus. According to certain embodiments, recombinant viral vectors can be administered directly to the skin of a subject (in vivo) or they can be administered to skin tissues or cells in vitro, and skin tissues or cells that were modified by the recombinant viruses may optionally be grafted or administered back to the subject (ex vivo). Conventional recombinant viral based vector systems can include retroviral, lentivirus, adenoviral, adeno-associated virus (AAV), vaccinia virus and herpes simplex virus vectors for gene transfer. Of these viral vectors, recombinant AAV is thought to be the safest due to its lack of pathogenicity. Integration in the host genome is possible with the retrovirus and lentivirus vector transfer methods, often resulting in long term expression of the inserted transgene. Additionally, high transduction efficiencies using these recombinant viruses have been observed in many different cell types and target tissues. In certain embodiments, following ultrasound treatment of the skin, rAAV vectors containing genes of interest are topically applied to the skin tissue and let passively diffuse to reach skin cells in both epidermal and dermal skin layers. The tropism of an AAV can be altered by different capsid proteins. A skilled in the art can select appropriate rAAV serotype, including serotypes 1-9 based on the tropism for a particular cell type. Table 1 shows a list of non-limiting target genes and their functions for skin gene therapy according to certain embodiments of the disclosure.

TABLE 1
A list of target genes.
1. Strengthen the dermis by ECM restructuring

| | |
|---|---|
| COL1A1 | Precursor for collagen type I |
| COL3A1 | Precursor for collagen type III |
| TIMP1 | Tissue MMP protease inhibitors1 |
| TIPM2 | Tissue MMP protease inhibitors2 |
| SMAD2 | Receptor-regulated Smad2 |
| SMAD3 | Receptor-regulated Smad3 |
| CTGF | Connective tissue growth factor |
| TGF-β1 | Transforming growth factor beta 1 |

2. Restore skin barrier function by targeting epidermal cell turnover
3. Prevent non-melanoma skin cancer by modulating tumor suppressor genes

| | |
|---|---|
| KRT6A | Keratin 6 |
| NOTCH1(icd) | Notch1 intracellular domain |
| TET2 | Tet methylcytosine dioxygenase 2 |
| TET3 | Tet methylcytosine dioxygenase 3 |

4. Improve metabolic state

| | |
|---|---|
| Sirt1 | Sirtuin 1 |
| Sirt6 | Sirtuin 6 |
| Pck2 | Phosphoenolpyruvate carboxykinase 1 |
| Pparg | Peroxisome proliferator activated |
| | receptor gamma |
| Cisd2 | CDGSH iron sulfur domain 2 |

5. Improve epidermal stem cell metabolism and reprogramming

| | |
|---|---|
| MDH1 | Malate dehydrogenase 1 |
| MDH2 | Malate dehydrogenase 2 |
| Aco1 | Aconitase 1 |
| Aco2 | Aconitase 2 |
| IDH1 | Isocitrate dehydrogenase 1 |
| IDH2 | Isocitrate dehydrogenase 2 |
| IDH3 | Isocitrate dehydrogenase 3 |
| ENO1 | Enolase 1 |
| GOT1 | Glutamic-Oxaloacetic Transaminase 1 |
| GOT2 | Glutamic-Oxaloacetic Transaminase 2 |
| MUC1 | Mucin 1 |
| MCU | Mitochondrial Calcium Uniporter |

Embodiments of the present disclosure contemplate delivery of nucleic acids encoding genes producing extracellular matrix proteins including but not limited to COL1A1, COL3A1, TIMP1, TIMP2, SMAD2, SMAD3, CTGF, TGF-beta1) (Table 1, 1). The disclosed methods contemplate combating the age-related alterations of the dermis; the largest portion of the skin. The bulk of the epidermis is composed of collagenous extracellular matrix, which confers mechanical strength, elasticity and resilience to the skin. These functions are failing in both chronologically aged and photo aged skin due to alterations in the expression levels of extracellular proteins. The disclosed methods contemplate restoring “youthfull” levels of the extracellular matrix to counteract aging defects.

Embodiments of the present disclosure further contemplate delivery of nucleic acids encoding genes controlling the proper epidermal differentiation and renewal (Table 1, 2).

Embodiments of the present disclosure also contemplate delivery of nucleic acids encoding genes including but not limited to KRT6A, NOTCH1(icd), TET2, and TET3 (Table 1, 3). Photoaged skin sustains more numerous than any other tissue insults to its DNA. As a consequence, skin undergoes “extrinsic aging”, which at molecular level is caused by the high mutational loads evinced in the epidermis of all healthy individuals as early as at the age of 40. The continued degradation (i.e. aging) of the skin is the major factor leading to easily observable changes in skin appearance and pigmentation, and on the other end of the spectrum to onsets of benign and malignant skin lesions such as seborrheic keratoses, actinic keratoses and non-melanoma skin cancer. The disclosed methods contemplate restoring the proper epidermal homeostasis in photoaged skin by delivering genes encoding wild type (not mutated) determinants of epidermal differentiation (Notch) and stem cell renewal (Krt6A, TET2/3).

Embodiments of the present disclosure also contemplate methods for reversing age related alterations in the skin (Table 1, 4 & 5). The disclosure provides for a gene therapy method for the delivery of nucleic acids encoding Sirt1, Sirt6, Pck1, Pparg, and Cisd2, MDH1/2, Aco1/2, IDH1/2/3, Enolase, GOT1/2, MUC1, and MCU. The metabolic state of epidermal progenitors is emerging as an important determinant of skin age. In the epidermis, stem cell's commitment to differentiation, triggered by an increase in intracellular calcium, corresponds to a critical metabolic switch from cytosolic glycolysis to mitochondrial oxidative phosphorylation (OXPHOS). Alterations in mitochondrial OXPHOS is associated with failure to maintain functioning “youth” epidermis. Importantly, the capacity to elevate mitochondrial respiration fails in aging epidermal stem cells simultaneously with decreased expression of rate-limiting mitochondrial enzymes. Thus, to combat the failure in the switch to mitochondrial OXPHOS at the onset of commitment to differentiation during skin aging, the disclosed methods contemplate delivering multiple genes affecting key metabolic pathways to reverse the effects of aging.

Different skin layers, structures and cells can be targeted for gene delivery according to certain embodiments of the disclosed methods. The skin is composed of diverse cells derived from three distinct embryonic origins: neurectoderm, mesoderm, and neural crest. Recombinant viral vectors can be delivered to one or more of the three layers of the skin: the epidermis, dermis, and hypodermis. The epidermis, the outermost layer, is primarily composed of stratified squamous epithelium of keratinocytes, which is derived from neurectoderm and comprises over ninety percent of epidermal cells. The stratified squamous epithelium is further divided into four layers, starting with the outermost layer: stratum corneum (SC), stratum granulosum (SG), stratum spinosum (SS), and stratum basale (SB). Cells of the epidermis including keratinocytes which are responsible for the cohesion of the epidermal structure and the barrier function, pigment-containing melanocytes, antigen-processing Langerhans cells, and pressure-sensing Merkel cells can be targeted by the viral vectors.

The dermis is a connective tissue that is responsible for the mechanical properties of the skin. It is composed of fibroblasts of mesoderm origin, which lie within an extracellular specialized matrix. Collagens are interwoven with elastin, proteoglycans, fibronectin, and other components. The epidermis and dermis are connected by a basement membrane that is composed of various integrins, laminins, collagens, and other proteins that play important roles in regulating epithelial-mesenchymal cross-talk. The superficial papillary dermis is arranged in ridge-like structures called the dermal papillae, which contains microvascular and neural networks and extends the surface area for these epithelial-mesenchymal interactions. Sebaceous glands, eccrine glands, apocrine glands and hair follicles are of neurectoderm origin and develop as downgrowths of the epidermis into the dermis. Outer root sheath of the hair follicle is contiguous with the basal epidermal layer. In addition, the dermis also contains blood vessels and lymphatic vessels of mesoderm origin, and sensory nerve endings of neural crest origin. The hypodermis, which is deep to the dermis, is composed primarily of adipose tissue of mesoderm origin, and separates the dermis from the underlying muscular fascia. Viral vectors can also target these cells, glands, and structures of the dermis and hypodermis.

Recombinant viral vectors can also target skin-specific stem cells which possess the ability for skin tissue to self-renew. Multipotent or unipotent skin stem cells are slowly-cycling cells that reside in at least five distinct niches in the skin: basal (innermost) layer of epidermis, hair follicle bulge, base of sebaceous gland, dermal papillae, and dermis. Not only are these stem cells critical for the long-term maintenance of the skin tissue but also are activated by wounding to proliferate and regenerate the tissue. Skin-specific stem cells include hair follicle stem cells for hair follicle and continual hair regeneration, melanocyte stem cells giving rise to the melanocytes in both the hair matrix and epidermis, stem cells at the base of the sebaceous gland for continually generating terminally differentiated sebocytes, which degenerate to release lipids and sebum through the hair canal and lubricate the skin surface, mesenchymal stem cells that giving rise to fibroblasts, nerves and adipocytes, and a skin-derived precursor stem cell (SKP) distinct from mesenchymal stem cells.

It is to be understood that the basic concepts of the present disclosure described herein are not limited by cell type. Further, cells include any in which it would be beneficial or desirable to regulate a target nucleic acid. Such cells may include those which are deficient in expression of a particular protein leading to a disease or detrimental condition of the skin. Such diseases or detrimental conditions are readily known to those of skill in the art. According to the present disclosure, the nucleic acid responsible for expressing the particular protein may be targeted by the methods described herein and a transcriptional activator resulting in upregulation of the target nucleic acid and corresponding expression of the particular protein. In this manner, the methods described herein provide therapeutic treatment. Such cells may include those which over express a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional repressor resulting in downregulation of the target nucleic acid and corresponding expression of the particular protein. In this manner, the methods described herein provide therapeutic treatment.

According to one aspect, the cell is a mammalian cell. According to one aspect, the cell is a human cell. According to one aspect, the cell is a stem cell whether adult or embryonic. According to one aspect, the cell is a pluripotent stem cell. According to one aspect, the cell is an induced pluripotent stem cell. According to one aspect, the cell is a human induced pluripotent stem cell. According to one aspect, the skin cell is in vitro, in vivo or ex vivo.

According to certain aspects, the skin tissue is in vivo, ex vivo, or in vitro. According to certain aspects, the skin tissue includes skin grafts, explants, artificial skin tissues and skin substitutes.

The skin tissues and cells can derive from a subject of a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Complex signaling pathways that control self-renewal, proliferation, and differentiation are critical for maintaining skin homeostasis and regeneration. The methods of the present disclosure are amenable for skin gene therapy and genome editing therapy that are feasible for modulate gene expression and genome editing of target molecules in the signaling pathways related to maintaining skin homeostasis and regeneration. According to certain aspects, recombinant viral vectors can be designed to combine with the CRISPR system for delivery of nucleic acid molecules that alter target genome and modulate target gene expression of skin cells. For example, the CRISPR type II system is a recent development that has been utilized for genome editing in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W. Y., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. eLife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR is the most well-characterized and widely used. The Cas9-gRNA complex first probes DNA for the protospacer-adjacent motif (PAM) sequence (−NGG for Sp Cas9), after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a double-strand break (DSB) that is predominantly repaired by the non-homologous end joining (NHEJ) pathway or, to a lesser extent, template-directed homologous recombination (HR). CRISPR methods are disclosed in U.S. Pat. Nos. 9,023,649 and 8,697,359. See also, Fu et al., Nature Biotechnology, Vol. 32, Number 3, pp. 279-284 (2014). Additional references describing CRISPR-Cas9 systems including nuclease null variants (dCas9) and nuclease null variants functionalized with effector domains such as transcriptional activation domains or repression domains include J. D. Sander and J. K. Joung, Nature biotechnology 32 (4), 347 (2014); P. D. Hsu, E. S. Lander, and F. Zhang, Cell 157 (6), 1262 (2014); L. S. Qi, M. H. Larson, L. A. Gilbert et al., Cell 152 (5), 1173 (2013); P. Mali, J. Aach, P. B. Stranges et al., Nature biotechnology 31 (9), 833 (2013); M. L. Maeder, S. J. Linder, V. M. Cascio et al., Nature methods 10 (10), 977 (2013); P. Perez-Pinera, D. D. Kocak, C. M. Vockley et al., Nature methods 10 (10), 973 (2013); L. A. Gilbert, M. H. Larson, L. Morsut et al., Cell 154 (2), 442 (2013); P. Mali, K. M. Esvelt, and G. M. Church, Nature methods 10 (10), 957 (2013); and K. M. Esvelt, P. Mali, J. L. Braff et al., Nature methods 10 (11), 1116 (2013).

The practice of the disclosed methods employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Skin diseases and conditions may be characterized by abnormal loss of expression or underexpression of a particular protein or abnormal gain or overexpression of a particular protein. Such skin diseases or conditions can be treated by upregulation or down regulation of the particular protein. Accordingly, methods of treating a skin disease or condition are provided where delivery of nucleic acid sequences via recombinant viruses to skin cells results in up- or down-regulation of expression of the target nucleic acid. One of skill in the art will readily identify such diseases and conditions based on the present disclosure. Examples of target proteins/polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected skin tissues compared with skin tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. Examples of disease-associated genes and polynucleotides of skin are available from McKusick-Nathans Institute of Genetic Medicine, Johns Hopkins University (Baltimore, Md.) and National Center for Biotechnology Information, National Library of Medicine (Bethesda, Md.), available on the World Wide Web. Mutations in these genes and pathways can result in production of improper proteins or proteins in improper amounts which affect skin function. Such genes, proteins and pathways may be the target polynucleotide of the disclosed methods.

Embodiments of the present disclosure provide methods for delivering foreign or heterologous nucleic acids (i.e. those which are not part of a cell's natural nucleic acid composition) encoding genes of interest into a cell of the skin tissue. In one embodiment, the skin tissue is pre-treated with ultrasound prior to deliver of foreign or heterologous nucleic acids. Alternative methods for introducing foreign or heterologous nucleic acids into cells can be used in combination with the delivery methods disclosed herein. These alternative methods are known to those skilled in the art including transfection, transduction, viral transduction, microinjection, lipofection, nucleofection, nanoparticle bombardment, transformation, conjugation and the like. One of skill in the art will readily understand and adapt such methods using readily identifiable literature sources.

Embodiments of the present disclosure provide methods for delivering vectors encoding genes of interest into a cell of the skin tissue. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” or “operatively linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). Vectors according to the present disclosure include those known in the art as being useful in delivering genetic material into a cell and would include regulators, promoters, nuclear localization signals (NLS), start codons, stop codons, a transgene etc., and any other genetic elements useful for integration and expression, as are known to those of skill in the art.

According to certain aspect, the present disclosure provides viral vectors for use in gene therapy methods disclosed herein and these viral vectors are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

According to certain aspects, the present disclosure provides methods of non-viral delivery for use in gene therapy methods disclosed herein. Methods for non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

According to some aspects, the present disclosure provides nucleic acid sequences encoding gene of interest including regulatory elements for optimum expression of the gene of interest in target cell or target tissue. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 0-actin promoter, the phosphoglycerol kinase (PGK) promoter, and the EF1a promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### Example I

### Materials and Methods

Recombinant AAV (rAVV) Vector A “recombinant parvoviral” or “AAV vector” or “rAAV vector” herein refers to a vector comprising one or more polynucleotide sequences of interest, genes of interest or “transgenes” that are flanked by at least one parvoviral or AAV inverted terminal repeat sequences (ITRs). Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions. Thus, in a further aspect the invention relates to a nucleic acid construct comprising a nucleotide sequence encoding a porphobilinogen deaminase as herein defined above, wherein the nucleic acid construct is a recombinant parvoviral or AAV vector and thus comprises at least one parvoviral or AAV ITR. Preferably, in the nucleic acid construct the nucleotide sequence encoding the porphobilinogen deaminase is flanked by parvoviral or AAV ITRs on either side.

The AAV VP proteins are known to determine the cellular tropicity of the AAV virion. The VP protein-encoding sequences are significantly less conserved than Rep proteins and genes among different AAV scrotypes. The ability of Rep and ITR sequences to cross-complement corresponding sequences of other serotypes allows for the production of pseudotyped rAAV particles comprising the capsid proteins of one serotype (e.g., AAV5) and the Rep and/or ITR sequences of another AAV serotype (e.g., AAV2). Such pseudotyped rAAV particles are a part of the present invention. Herein, a pseudotyped rAAV particle or hybrid rAAV may be referred to as being of the type “x/y”, where “x” indicates the source of ITRs and “y” indicates the serotype of capsid, for example a 2/5 rAAV particle has ITRs from AAV2 and a capsid from AAV5. Modified “AAV” sequences also can be used in the context of the present disclosure, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid. sequence identity (e.g., a sequence having from about 75% to about 99% nucleotide sequence identity) to an AAV1, AAV2, AAV3, AAV4, AAV5, AAV6, AAV7, AAV8 or AAV9 ITR, Rep, or VP can be used in place of wild-type AAV ITR, Rep, or VP sequences. Preferred adenoviral vectors are modified to reduce the host response. See, e.g., Russell (2000) J. Gen. Virol. 81:2573-2604; US patent publication no. 20080008690; and Zaldumbide et al. (2008) Gene Therapy 15(4):239-46; all publications incorporated herein by reference.

### AAV Vector and Expression Cassette

The schematic of the backbone vector is as follows:

(SEQ ID NO: 32)
WPRE3_SV40 Late
polyA_ITR_florigin_AmpR_pBR322origin_ITR_shEf1a.
(SEQ ID NO: 33)
Forward primer 5′-ATGTTAGCGGCCGCGCCACCATGATGAGCTTT
GTGCAAAAGGGGAGC
and
(SEQ ID NO: 34)
reverse primer 5′-CTTACGGCTAGCTTATTATAAAAAGCAAACAG
GGCCAACGTCCAC

were used to amplify COL3A1 gene sequence. The bold and italicized part of the forward primer is the Kozak sequence. The bold and italicized part of the reverse primer is the stop codon sequence. The two parts were combined and used in PCR to amplify the COL3A1 sequence. The underlined sequences in both the forward and reverse primers are overhangs attached during PCR to create a fusion COL3A1 sequence for a total length of 3526 base pairs. After restriction digest using unique restriction enzyme site overhangs NotI and NheI, the backbone vector and gene were ligated together.

### AAV Production

The method of AAV production and titer quantification was carried out according to Lock, M. 2010 Human gene therapy; Kwon, O. et al., (2010) J Histochem Cytochem. 58(8):687-694. Briefly, Hek293 cells were triple co-transfected at 75% confluency in one 10 layer Nunc™ Cell Factory™ System from Thermo Scientific (Rockford, Ill.) using PEI transfection reagent following manufacturer's instructions. Cells and supernatant were harvested separately after 72 hours post transfection. The cells were spun down and lysed with 3 freeze-thaw cycles and incubated with Benzonase (E1015-25KU, Sigma). They were then clarified by spinning at 10,500×G for 20 min and the supernatant was added to the rest of the media supernatant. Everything was filtered through a 0.2 uM filter and was then concentrated using lab scale TFF system (EMD Chemicals, Gibbstown, N.J.) down to 15 ml. We used a Pellicon XL 100 kDa filter and followed manufactures instructions (EMD Chemicals, Gibbstown, N.J.). The concentrated prep was re-clarified by centrifugation at 10,500 Å˜g and 15° C. for 20 min and the supernatant was carefully removed to a new tube. Six iodixanol step gradients were formed according to the method of Zolotukhin and colleagues. See Zolotukhin S., (1999) Gene Ther. 6:973-85, with some modifications as follows: Increasingly dense iodixanol (OptiPrep; Sigma-Aldrich, St Louis, Mo.) solutions in phosphate-buffered saline (PBS) containing 10 mM magnesium chloride and 25 mM potassium were successively underlaid in 39 ml of 62 Quick-Seal centrifuge tubes (Beckman Instruments, Palo Alto, Calif.). The steps of the gradient were 4 ml of 15%, 9 ml of 25%, 9 ml of 40%, and 5 ml of 54% iodixanol. Fourteen milliliters of the clarified feedstock was then overlaid onto the gradient and the tube was sealed. The tubes were centrifuged for 70 min at 242,000 Å˜g in a VTi 50 rotor (Beckman Instruments) at 18° C. and the 40% gradient was collected through an 18-gauge needle inserted horizontally at the 54%/40% interface. The virus containing iodixanol was diafiltered using Amicon 15-Ultra and washed 5 times with final formulation buffer (PBS-35 mM NaCl), and concentrated to 1 ml.

### Vector Characterization

DNase I-resistant vector genomes were titered by TaqMan PCR amplification (AppliedBiosystems, Foster City, Calif.), using primers and probes directed against the WPRE3 poly Adenylation signal encoded in the transgene cassette. The purity of gradient fractions and final vector lots were evaluated by sodium dodecyl sulfate-polyacrylamide gel electrophoresis (SDS-PAGE) and the proteins were visualized by SYPRO ruby staining (Invitrogen) and UV excitation.

### Ultrasound Treatment and Delivery

Skin samples were mounted in custom diffusion chambers. Immediately before administration, the donor chamber was filled with 1.5 mL of phosphate buffered saline (PBS). 20 kHz ultrasound was utilized to maximize transient cavitation events, which have previously been shown to be the primary mechanism of enhancement. 20 kHz ultrasound was generated with a 12-element probe (probes 9 mm diameter) driven by a VCX 500 (Sonics and Materials, Inc., Newtown, Conn.). For all applications, the ultrasound probe tip was placed 3 mm away from the surface of the tissue. Ultrasound intensities were calibrated by calorimetry to 5 W/cm². Ultrasound was applied using a 50% duty cycle (5 s on, 5 s off). After administration, the PBS was removed. A solution (10 μl) of AAV was the applied topically on the skin and incubated at 32° C. for 60 minutes in the skin explant experiments, and 5 minutes in the hairless mice experiments. The effective dose range for small animals (mice), skin explants, and reconstructed human skin, following skin permeation by ultrasound, is between 5×10⁸ and 1×10¹² genome copy (gc)/cm². 5×10⁹ and 5×10¹⁰ were used for low and high dose, respectively.

### Image Processing Algorithm

The signal of EGFP in frozen samples from skin explants (16 mm×1 mm×20 μm) were analyzed and quantified using a custom MatLab scripts for image post-processing. The algorithm executes flat-field and background corrections and creates a logical mask of the image. Based on the different emission spectra of tissue auto-fluorescence and signal due to the expression of EGFP, the algorithm performs linear un-mixing of the total fluorescence intensity. Finally, it identified the areas of these unmixed signals of background autofluorescence and GFP signal.

### Histology and Immunofluorescence

Mouse or human skin was harvested and fixed in 10% formalin or 4% paraformaldehyde overnight for frozen sections, respectively. Frozen sections were used for Hematoxylin and Eosin (H&E) staining and histological analysis. H&E staining was carried out following the standard protocol (http://www.ihcworkd.com). Slides were mounted in Entellan New rapid mounting media (Electron Microscopy Sciences). Frozen sections (mounted in OCT embedding compound and frozen at −80 C) were used for immunofluorescence staining: primary antibodies were incubated for 3 hours, and second antibodies were incubated for 1 hour at room temperature in 5% BSA/PBST. Nuclei were stained with DAPI (Invitrogen), and the slides were mounted in Prolong Gold Antifade Mount (Invitrogen). Primary antibodies were used at 1:200 dilution, while secondary antibodies at 1:1000.

### Organ Cultures

Discarded human skin samples from abdominoplasty and/or breast reduction procedures were obtained from Massachusetts General Hospital (Boston, Mass.) under patients' agreement and institutional approval. Skin samples, sterilized in 70% ethanol and cut, after removal of subcutaneous fat, into 1.6 cm diameter pieces, were placed in keratinocyte serum-free medium (KSF, GIBCO-BRL) supplemented with epidermal growth factor (EGF) and bovine pituitary extract (BPE), in 0.25% agar (Sigma). The epidermis was maintained at the air-medium interface. For RNA and protein collection, skin samples were chilled (on ice) and homogenized using PRO200 BIO-GEN tissue homogenizer (Pro Scientific Inc., Oxford, Conn.).

### Western Blot

Protein from all tissues was isolated with RIPA (radioimmunoprecipitation assay) buffer containing protease and phosphatase inhibitors (all reagents purchased from Boston BioProducts, Ashland, Mass.). All specimens were chopped in small pieces and disrupted by PRO200 BIO-GEN tissue homogenizer (Pro Scientific Inc., Oxford, Conn.). Protein concentration in the clear lysates after centrifugation was measured with the Pierce BCA Protein Assay (Pierce Biotechnology, Grand Island, N.Y.). Western blots were quantified using the Fiji image processing software (open-source tool by ImageJ, https://imagej.nih.gov/ij/).

### RT-qPCR

One-step TaqMan (AppliedBiosystems, Foster City, Calif.) RT-qPCR were used with primers and probes directed against human COL3A1 encoded in the transgene cassette to perform quantification for gene expression. ACTB gene was used to quantify reference levels in the RNA samples. Equal amounts (as quantified by Agilent's bioanalyzer) of total RNA were used as input for all gene expression measurements.

### Tissue Harvest

Skin is immediately harvested after euthanasia. Part of it was snap frozen in dry ice for qPCR/qRT-PCR analysis and RNA/DNA-sequencing and the other part of each organ was then PFA-fixed for 3-24 hours depending on size and frozen in OCT buffer in liquid nitrogen bath for sectioning and analysis.

### Animal Euthanasia

Animals are euthanized by the slow fill method of CO₂ administration according to the equipment available in the facility. Typically, animals are euthanized in the home cage out of view from other animals. A regulator is used to ensure the proper flow rate. Animals should lose consciousness rapidly ˜30 sec. At the cessation of breathing (several minutes) animals will undergo a secondary physical method of euthanasia.

### Example II

### Efficacy of Gene Delivery

Most of the presently existing delivery methods for skin gene therapies rely on systemic delivery, electroporation or μ-needle injections. The delivery methods for skin gene therapy disclosed herein contemplate intra-dermal or topical delivery of recombinant viruses in a highly targeted and completely non-invasive manner. The ultrasound pre-treatment described herein is recognized to result in no pain or distress.

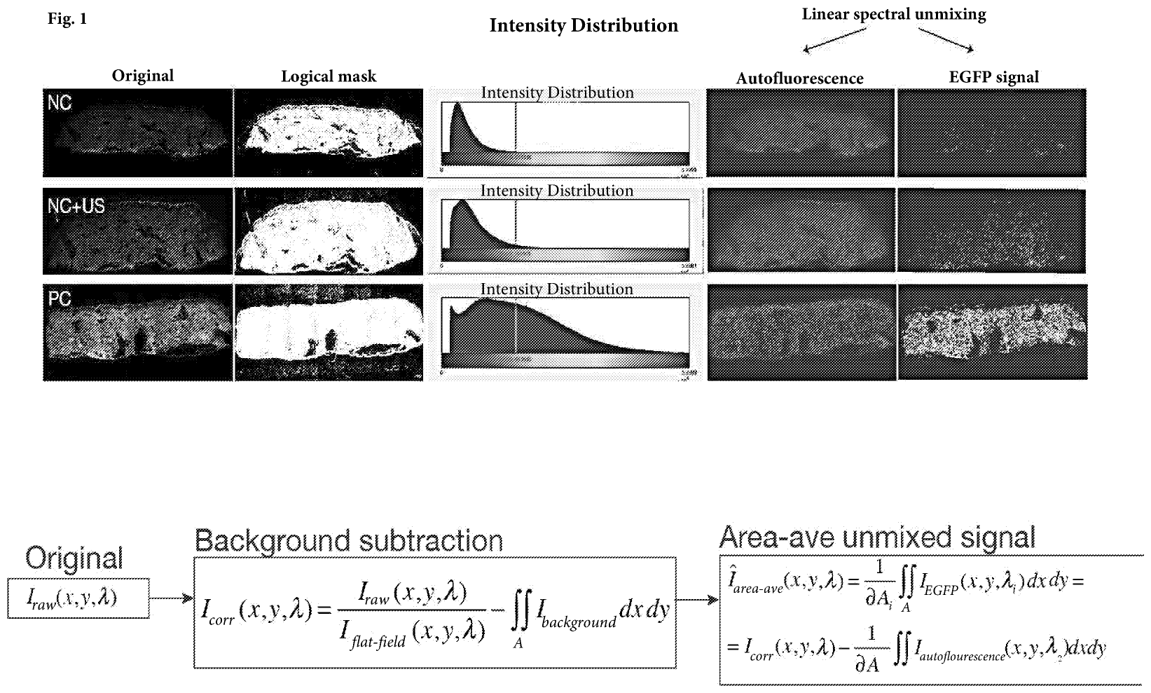

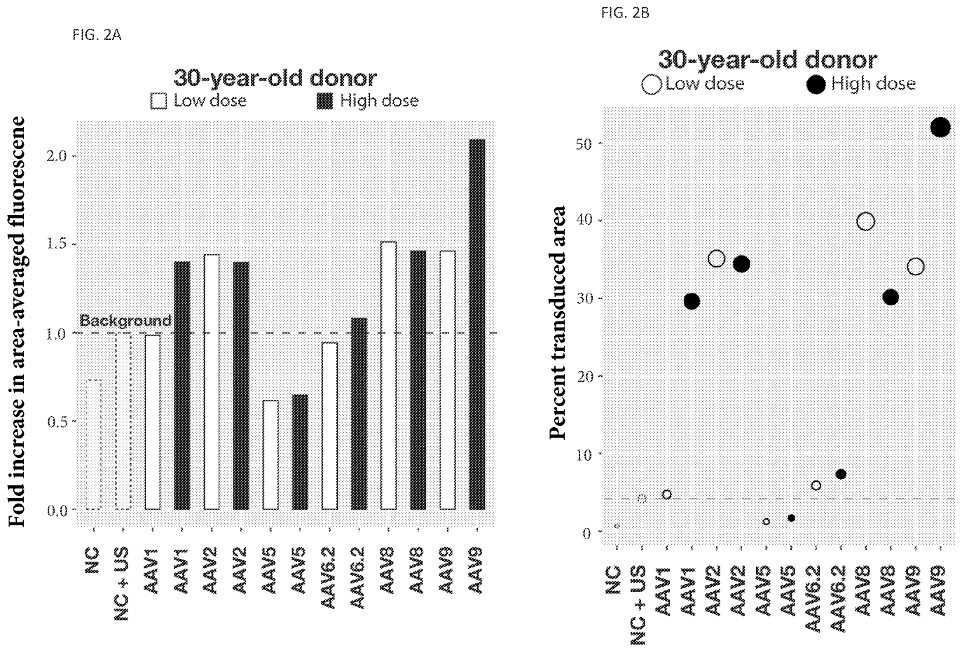

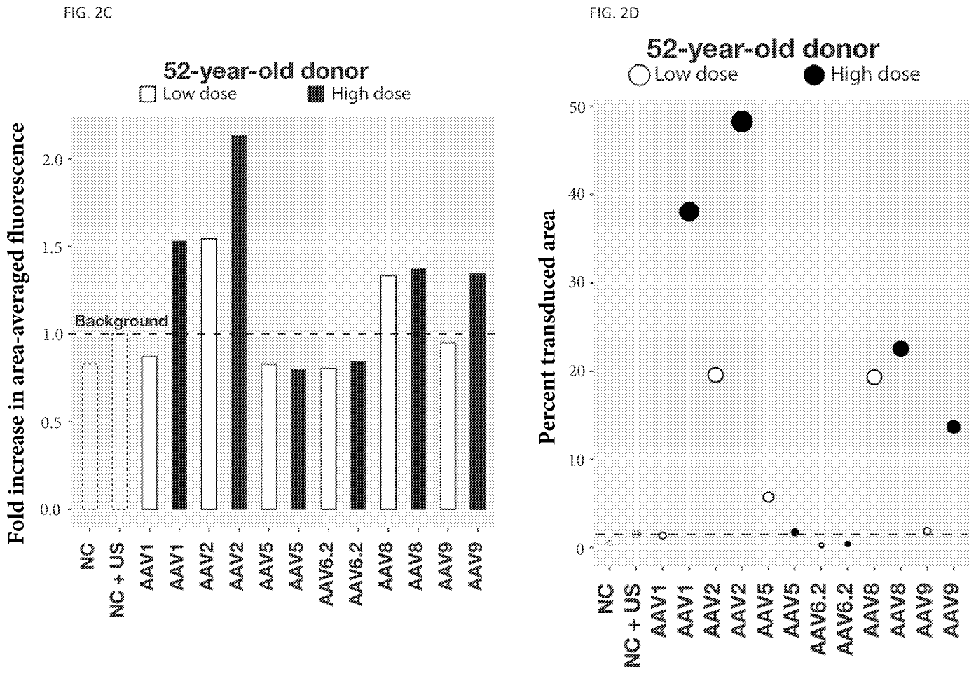

To determine delivery efficiency, the native fluorescence of a reporter gene distributed over a large surface area in full thickness human (breast) skin tissues cultured for 24 hours post-ultrasound treatment was studied. The reporter gene is enhanced GFP, and it is packaged in rAAV. The signal of EGFP in frozen samples (16 mm×1 mm×20 μm) was analyzed and quantified using a custom MatLab code for image post-processing. Briefly, the algorithm executes flat-field and background corrections and creates a logical mask of the image. Based on the different emission spectra of tissue auto-fluorescence and signal due to the expression of EGFP, the algorithm performs linear un-mixing of the total fluorescence intensity. Finally, it identified the areas of these unmixed signals. In FIG. 1, an example of this process was shown for one negative (no virus no ultrasound, NC), one negative ultrasound-treated (no virus, NC+US), and one positive controls (virus-EGFP+ultrasound, PC). In two donors of ages 30 and 52, signal intensities (of treated (virus-EGFP+ultrasound) and untreated (no virus, no ultrasound) skin explants) were reported in FIGS. 2A & 2C and percent transduced tissue areas were calculated and shown in FIGS. 2B & 2D for wild type rAAV2 with EGFP, and hybrid constructs encoding for the Rep gene from AAV2 and Cap gene derived from serotypes AAV1, AAV5, AAV6.2, AAV8, and AAV9 (AAV2/1, AAV2/5, AAV2/6.2, AAV8/2 and AAV9/2). Cell tropism of these hybrid viruses and wild-type AAV2 considerably differs in whole skin tissues and only certain viral capsids displayed successful transductions. It was shown that up to 2.1-fold enhancement in the expression of EGFP and 40-50% of infectivity in surface area (Between ultrasound treated vs. untreated across different hybrid viruses.)

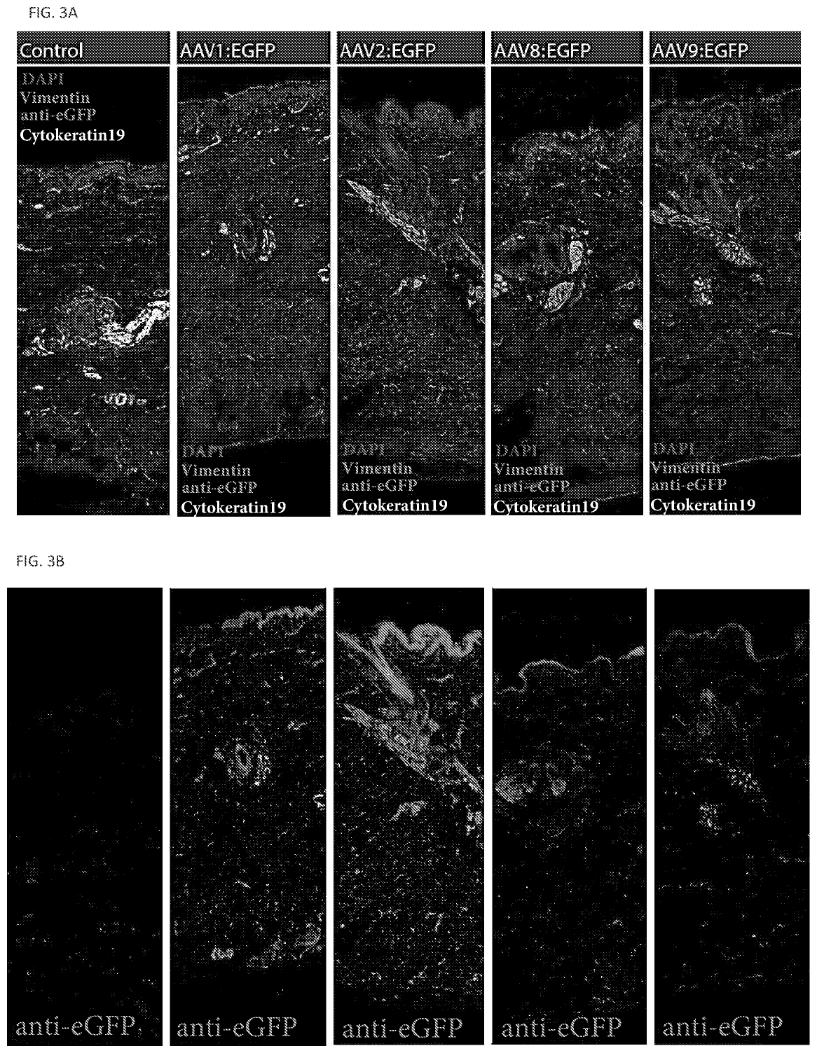

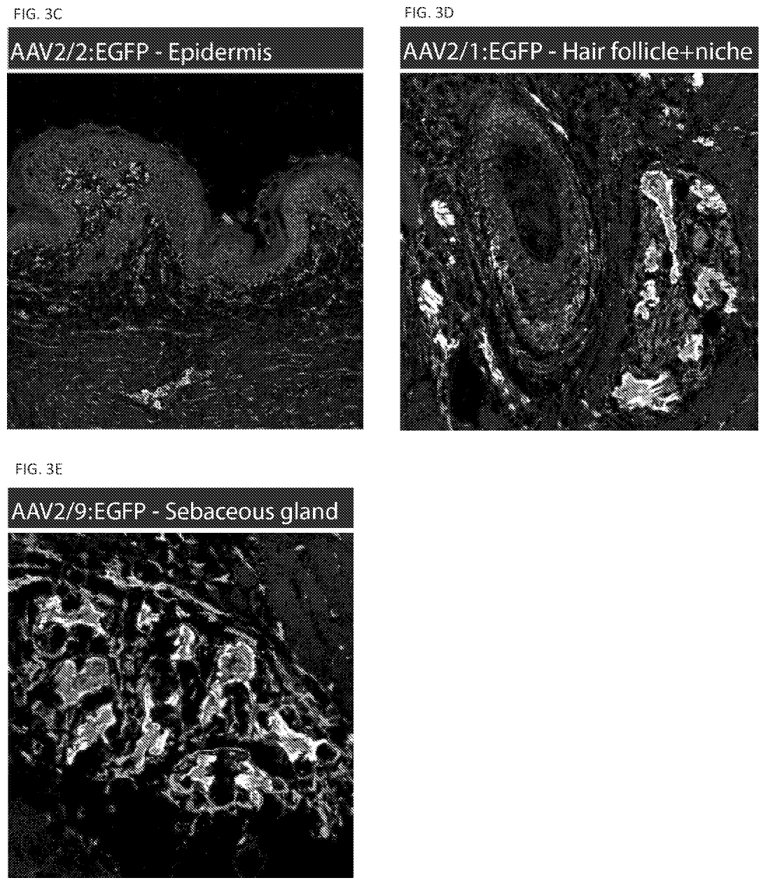

Based on the results described in FIGS. 2A-2D, samples were selected that were infected with pRep2/Cap1: EGFP, wild-type AAV2 (pRep2/Cap2: EGFP), pRep2/Cap8: EGFP, and pRep2/Cap9: EGFP to further analyze using confocal imaging of immunofluorescent staining. The tissues were stained with Vimentin (a fibroblast marker), anti-EGFP (a marker for the reporter gene), and Cytokeratin 19 (a marker for epithelial progenitors) (FIGS. 3A-3E). The findings are qualitatively summarized in Table 2 below.

TABLE 2
Qualitative cell tropism in human ex vivo experiments.

| | pRep2/Cap1 | pRep2/Cap2 | pRep2/Cap8 | pRep2/Cap9 |
|---|---|---|---|---|
| Skin feature | (AAV2/1) | (AAV2/2) | (AAV2/8) | (AAV2/9) |
| Keratinocytes | Moderate | Strong | Weak | Weak |
| Stem cells | Strong | Strong | Strong | Strong |
| (epithelial) | | | | |
| Fibroblasts | Moderate | Strong | Weak | Weak |
| Sebaceous | Strong | Strong | Strong | Strong |
| glands | | | | |
| Hair follicles | Strong | Strong | Strong | Moderate |

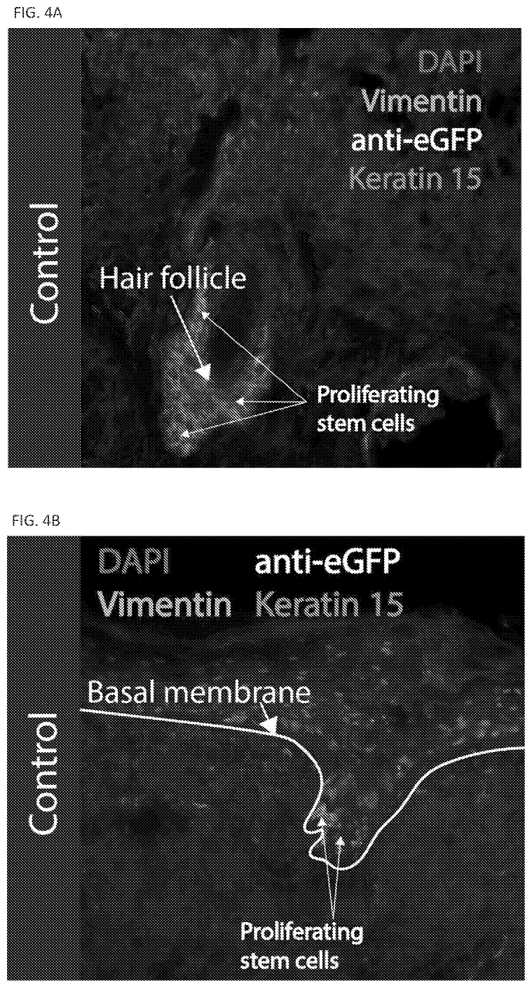

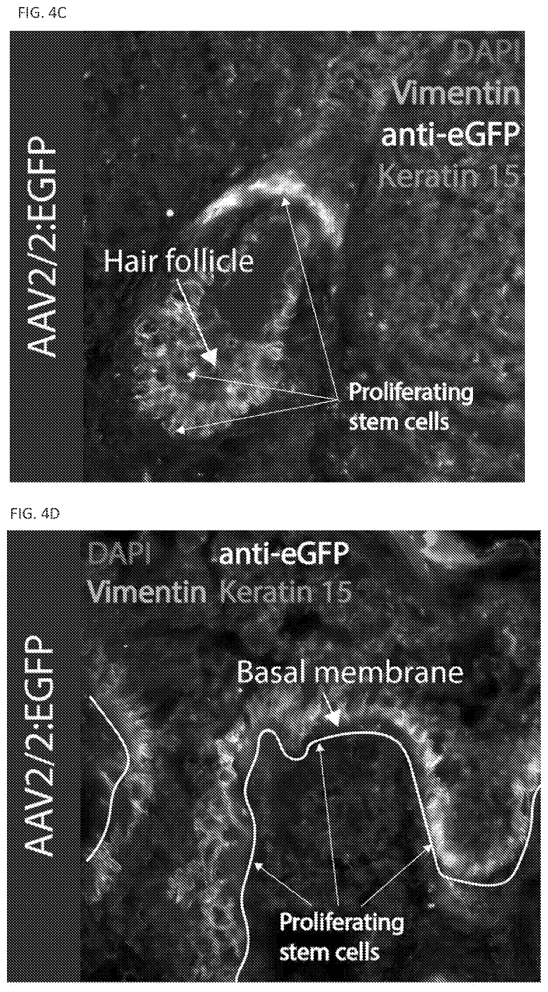

Because wild type AAV2 shows best gene delivery across all skin major cell types, rAAV2/2 was adopted to perform another set of tests in human skin explants (ex vivo) taken from the forehead skin of a 60-year-old donor. For cell tropism analysis, these tissues were stained for Vimentin (a fibroblast marker), anti-EGFP (a marker for the reporter gene), and Keratin 15 (a marker for epithelial stem cells) (FIGS. 4A-4D). Successful gene transfer was observed to all cells positive for Keratin 15—an epithelial stem cell marker against proliferating progenitors residing in the basement membrane of the dermal epidermal junction, hair follicles, and their niche (red and white overlapping signal in FIGS. 4C and 4D).

### Gene and Protein Modulation

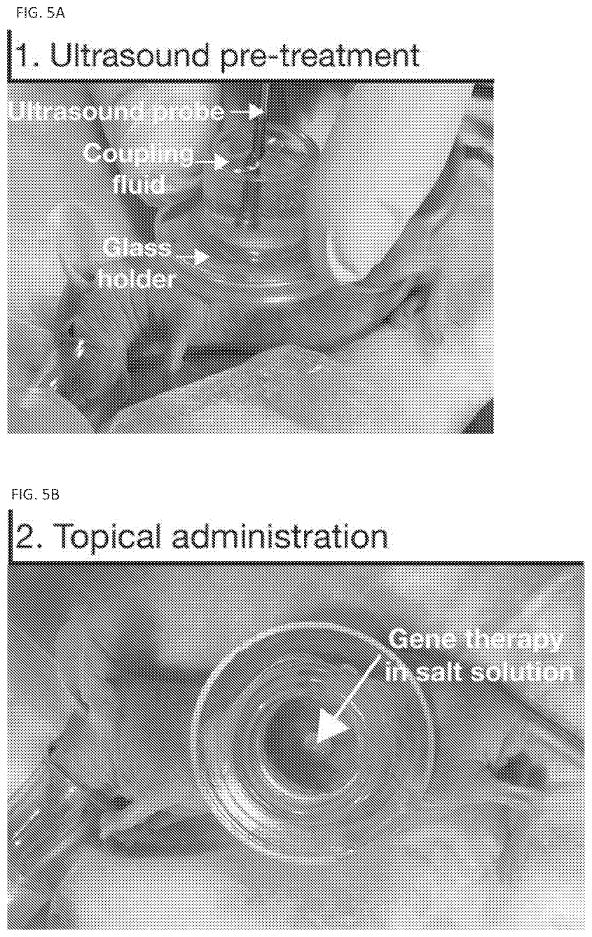

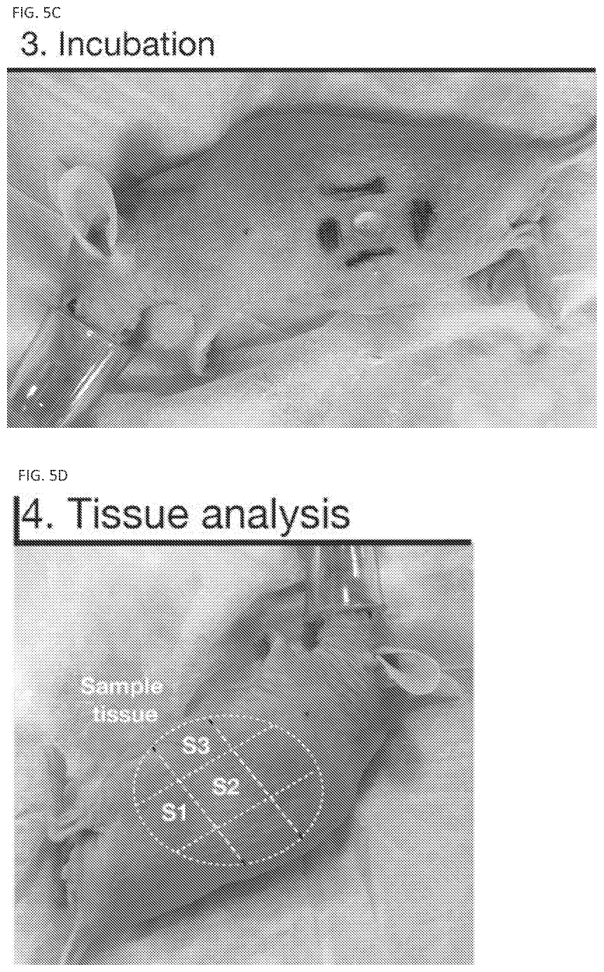

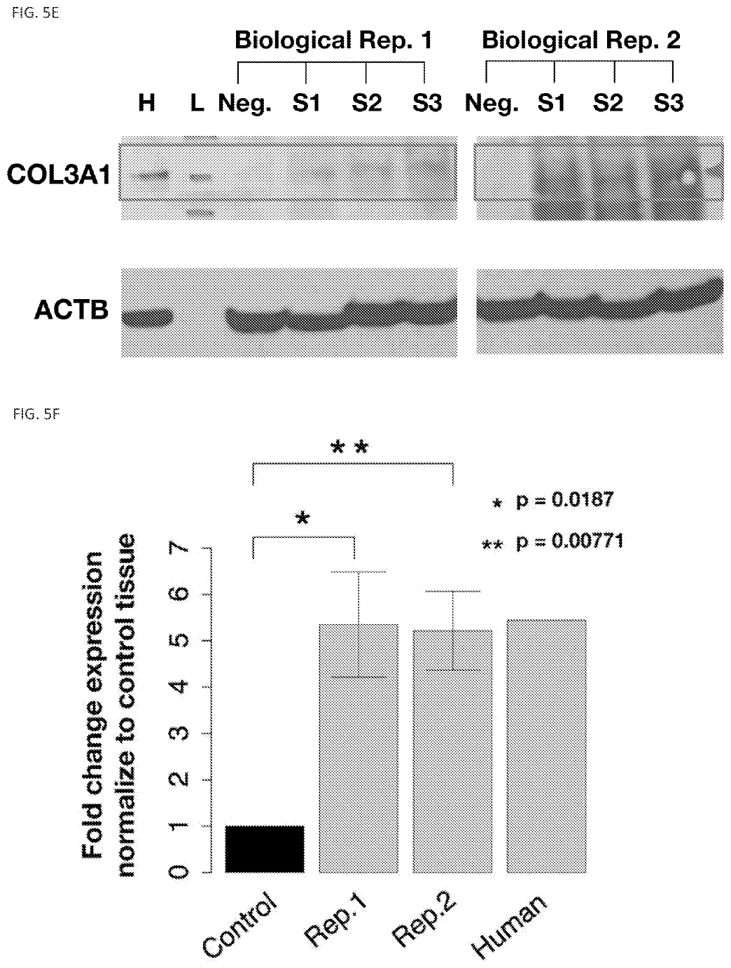

Next, to determine prolonged gene expression and stable protein modulation in vivo, hairless mice model was used. For these proof-of-concept experiments, we chose to deliver a single precursor gene to human collagen III (alpha domain), packaged in rAAV2 (rAAV2/2: COL3A1). Type III collagen is a human gene which encodes for collagen III fibrils which serve as a major component of the skin extracellular matrix, thus being an important target for the purposes of rebuilding aged- and diseased-skin dermis. Protein analysis and quantification using Western blot showed up to 5.4-fold over expression of collagen III, levels comparable to those in human skin (FIGS. 5A-5F). Type III collagen is primarily produced by dermal fibroblast cells. Collagen III is an attractive target for proof of principle experiments because hairless mice don't have this collagen type, which allows for zero-background detection of overexpression that is well-detectable by a human-specific antibody assay. We here compare the amounts of produced protein from overexpression of COL3A1 in hairless mice to the native amounts of protein encoded by COL3A1 in human skin. As a reference control, we used a tissue lysate prepared from a skin explant of an 18-year old individual.

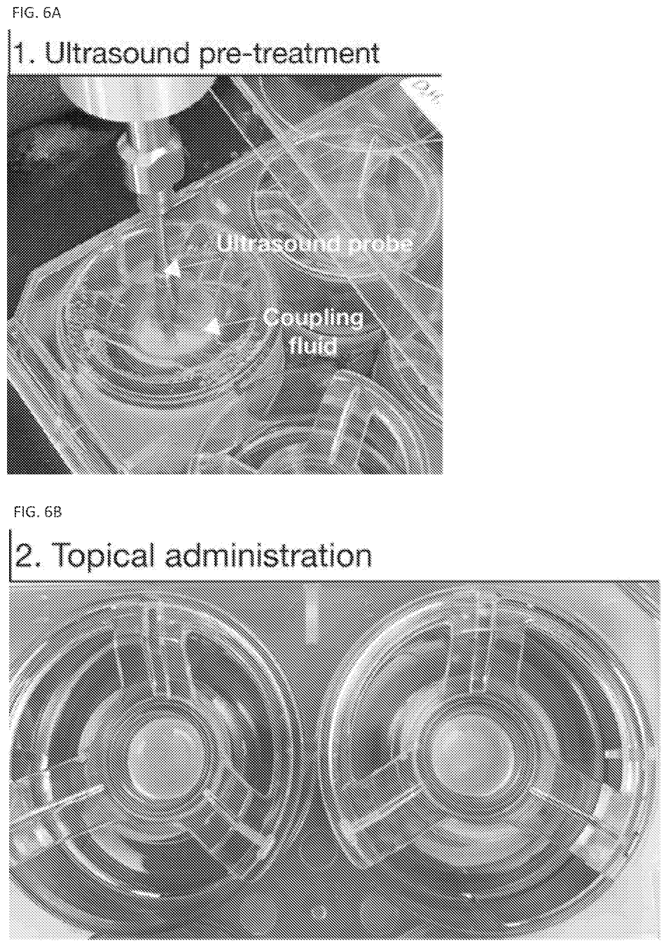

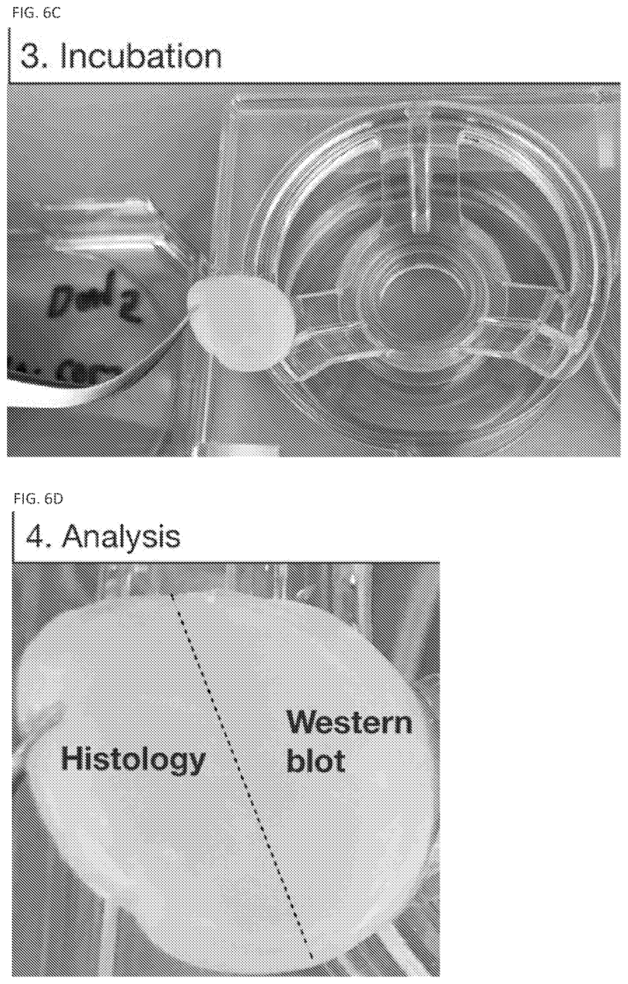

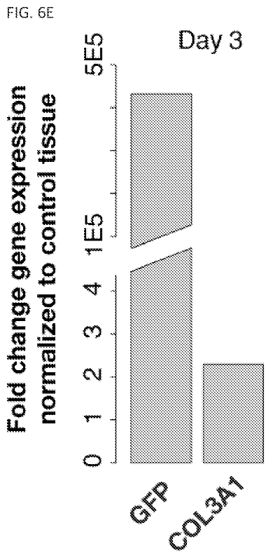

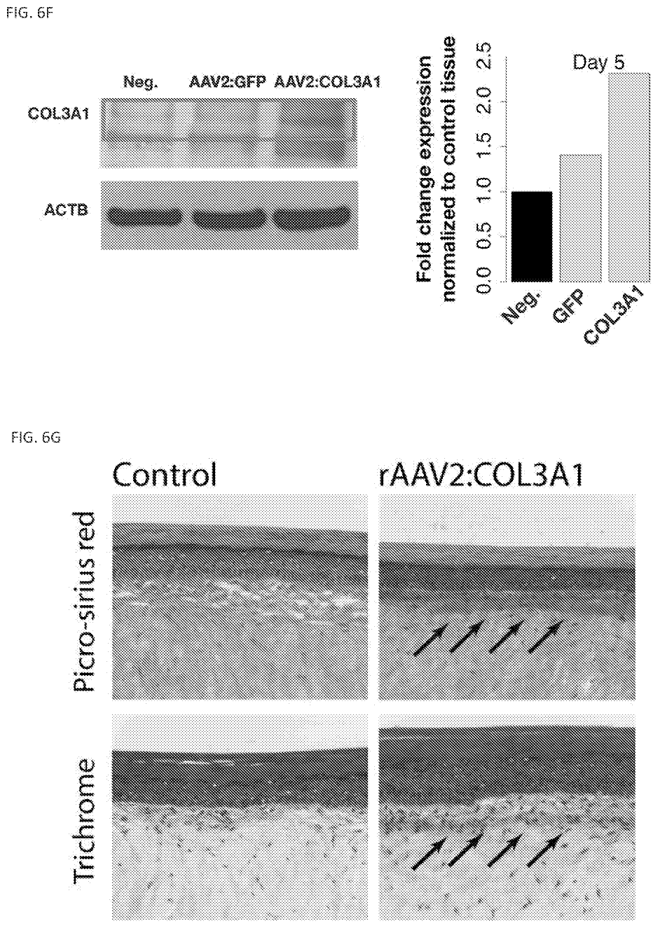

Similar to the in vivo mouse experiments, gene and protein modulation of collagen III was determined in vitro in human artificial skin (EpiDermFT tissues commercially made available by MatTek, Inc.). These artificial skin tissues contain primary human dermal fibroblasts and epidermal keratinocytes that are cultured to form a full thickness multilayer model of human skin. The tissue layers are metabolically and mitotically active and mimic in vivo characteristics. We delivered viral vectors of rAAV2/2: EGFP and rAAV2/2: COL3A1, and measured 40,000-fold and 2.8-fold increase in the gene expression of EGFP and COL3A1 via RT-qPCR. The reported gene expression fold increase is relative to untreated (no-AAV, no ultrasound) control samples. All gene expression levels are normalized to the expression of ACTB. Measured by Western blotting, collagen levels showed 2.3-fold enhancement relative to untreated negative control tissue. As another end-point, we confirmed collagen accumulation by histological analysis using Picro-sirius red and trichrome staining which quantify total collagen content (FIGS. 6A-6G).

| Nucleic Acid Sequences | |
|---|---|
| 1. COL1A1 chimeric DNA, Collagen Type I Alpha 1 Chain | |
| (SEQ ID NO: 1) | |
| 1 | cccacgcgtc cggactagtt ctagatcgcg agcggcccgg agttggggcg ccttgccccg |
| 61 | ggccccccag catgaagacc ccggcggaca cagggtttgc cttcccagat tgggcctaca |
| 121 | aaccggagtc atcccctggc tccaggcaga tccagctgtg gcactttatc ctggagctgc |
| 181 | ttcggaaaga ggagtaccag ggcgtcatcg cttggcaggg ggactacggg gagtttgtca |
| 241 | tcaaggaccc tggtgaacct ggcgagcctg gcggttcagg tccaatgggt ccccgaggtc |
| 301 | cccctggccc tcctggcaag aatggagatg atggggaagc tggcaagccc ggccgtcctg |
| 361 | gtgagcgtgg acctcctgga cctcagggtg ctcgtggatt gcctggaaca gctggcctcc |
| 421 | ctggaatgaa gggacaccga ggcttcagtg gtttggatgg tgccaaagga gatgctggtc |
| 481 | ctgctggtcc taagggagag cccggcagtc ctggtgaaaa cggagctcct ggccagatgg |
| 541 | gtccccgagg tctgcccggt gagagaggtc gccctggacc tcctggcact gctggtgctc |
| 601 | gcggtaacga tggtgctgtt ggtgctgctg gaccccctgg tcccaccggc cccactggcc |
| 661 | ctcctggctt ccctggtgca gttggtgcta agggtgaagc tggtccccaa ggagctagag |
| 721 | gctctgaagg tccccagggt gtgcgtggtg agcccggacc ccctggccct gctggtgctg |
| 781 | ccggccctgc tggaaaccct ggtgctgatg gacaacctgg cgctaaaggt gccaatggtg |
| 841 | ctcctggtat tgctggtgct cctggcttcc ctggtgcccg aggcccctct ggaccccagg |
| 901 | gccccagcgg ccctccaggt cccaagggta acagtggtga acctggtgct cctggcaaca |
| 961 | aaggagacac tggtgccaaa ggagaacccg gtgctactgg agttcaaggt cccccaggcc |
| 1021 | ctgccggaga agaaggaaaa cgaggagccc gtggtgagcc tggaccttcc ggactgcctg |
| 1081 | gacctcctgg cgagcgtggt ggacctggta gccgtggttt ccctggtgct gatggtgttg |
| 1141 | ctggccccaa gggtccttcc ggtgaacgtg gtgctcccgg acctgctggt cccaaaggtt |
| 1201 | ctcctggtga agctggtcgc cccggtgaag ctggtctccc tggtgccaag ggtctcactg |
| 1261 | gcagtcctgg cagccctggt cctgatggca aaaccggccc ccctggtccc gctggtcaag |
| 1321 | atggtcgccc tggacccgca ggtcctcctg gagcccgtgg ccaggctggt gtgatgggat |
| 1381 | tccctggacc taagggtacc gctggagaac ctggaaaggc tggagagcga ggccttcccg |
| 1441 | gaccccctgg cgctgttggt cctgctggca aagatggaga agctggagct cagggagccc |
| 1501 | ctggccctgc tggtcctgct ggtgagagag gtgaacaagg tcccgctggc tcccctggat |
| 1561 | tccagggtct tcctggtcct gccggtcctc ctggtgaagc aggcaagcct ggtgaacagg |
| 1621 | gtgttcctgg agaccttggt gcccctggac cctctggcgc aagaggcgag agaggtttcc |
| 1681 | ctggtgaacg tggtgtacaa ggtcccccag gtcctgctgg tccccgagga aacaatggtg |
| 1741 | cccccggcaa cgatggtgcc aagggtgata ctggtgcccc cggagctccc ggtagccagg |
| 1801 | gtgcccccgg tcttcaggga atgcctggtg aacgtggtgc agctggtctt ccaggtccta |
| 1861 | agggtgacag aggtgatgct ggtcccaaag gtgctgatgg ttctcctggt aaagatggtg |
| 1921 | cccgtggtct gactggtccc attggtcctc ctggccctgc tggtgcccct ggtgacaagg |
| 1981 | gtgaagctgg tcccagtggt cctcccggtc ccaccggagc ccgtggtgct cccggagacc |
| 2041 | gtggtgaggc tggtccccct ggtcctgctg gctttgccgg cccccctggt gctgatggcc |
| 2101 | aacctggtgc gaaaggtgaa cctggtgata ctggtgttaa aggtgatgct ggtcctcctg |
| 2161 | gccctgctgg tcctgctgga ccccccggcc ccattggtaa cgttggtgct cctggaccca |
| 2221 | aaggtcctcg tggtgctgct ggtccccctg gtgctactgg cttccctggt gctgctggcc |
| 2281 | gtgtcggtcc ccctggtccc tctggaaatg ctggaccccc tggccctccc ggtcccgttg |
| 2341 | gcaaagaagg gggcaaaggt ccccgtggtg agactggccc tgctggacgt cctggtgaag |
| 2401 | ttggtccccc aggtcccccc ggtcctgctg gtgagaaagg atctcctggt gctgatggac |
| 2461 | ctgctggctc tcctggtacc cctggacctc agggtattgc tggacaacgt ggtgtggtcg |
| 2521 | gtcttcccgg tcagagagga gaaagaggct tccctggtct tcctggcccc tctggtgaac |
| 2581 | ctggcaaaca aggtccttct ggatcaagtg gtgaacgcgg tccccctggc cccatggggc |
| 2641 | cccctggatt ggctggtccc cctggtgaat ctggacgtga gggatcccct ggtgctgaag |
| 2701 | gctcccctgg aagggatggt gctcccgggg ccaagggtga ccgtggtgag actggccctg |
| 2761 | ctggcccccc tggtgcccct ggtgctcccg gtgctcccgg ccctgttggt cccgctggca |
| 2821 | agaatggcga tcgtggtgag actggtcctg ctggtcctgc tggtcccatt ggccctgctg |
| 2881 | gtgcccgtgg ccctgctgga ccccaaggcc cccgtggtga caagggtgag acaggcgaac |
| 2941 | aaggtgacag aggcataaag ggtcatcgtg gcttctctgg tctccagggt cctcctggtt |
| 3001 | ctcctggttc tcctggtgaa caaggcccct ctggagcttc aggtcctgca ggcccccggg |
| 3061 | gtccccctgg ctctgctggt tctcctggca aagacggact caacggtctc cctggcccca |
| 3121 | ttggtccccc tggtcctcga ggtcgcactg gtgacagcgg ccctgctggt ccccccggcc |
| 3181 | ctcctggacc ccctggccct cctggacctc ccagtggcgg ttatgacttc agcttcctgc |
| 3241 | ctcagccacc tcaagagaag tctcaagatg gtggccgcta ctaccgggcc gatgatgcta |
| 3301 | acgtggttcg tgaccgtgac cttgaggtgg acaccaccct caagagcctg agtcagcaga |
| 3361 | ttgagaacat ccgcagcccc gaaggcagcc gcaagaaccc tgcccgcaca tgccgcgacc |
| 3421 | tcaagatgtg ccactctgac tggaagagcg gagagtactg gatcgaccct aaccaaggct |
| 3481 | gcaacctgga cgccatcaag gtctactgca acatggagac aggtcagacc tgtgtgttcc |
| 3541 | ctactcagcc gtctgtgcct cagaagaact ggtacatcag cccgaacccc aaggaaaaga |
| 3601 | agcacgtctg gtttggagag agcatgaccg atggattccc gttcgagtac ggaagcgagg |
| 3661 | gctccgaccc cgccgatgtc gctatccagc tgaccttcct gcgcctaatg tccaccgagg |
| 3721 | cctcccagaa catcacctat cactgcaaga acagcgtagc ctacatggac cagcagactg |
| 3781 | gcaacctcaa gaaggccctg ctcctccagg gatccaacga gatcgagctc agaggcgaag |
| 3841 | gcaacagtcg cttcacctac agcacccttg tggacggctg cacgagtcac accggaactt |
| 3901 | ggggcaagac agtcatcgaa tacaaaacca ccaagacctc ccgcctgccc atcatcgatg |
| 3961 | tggctccctt ggacattggt gccccagacc aggaattcgg actagacatt ggccctgcct |
| 4021 | gcttcgtgta aactccctcc accccaatct ggttccctcc cacccagccc acttttcccc |
| 4081 | aaccctggaa acagacgaac aacccaaact caatttcccc caaaagccaa aaatatggga |
| 4141 | gataatttca catggacttt ggaaaacatt ttttttcctt tgcattcacc tttcaaactt |
| 4201 | agtttttacc tttgaccaac tgaacgtgac caaaaaccaa aagtgcattc aaccttacca |
| 4261 | aaaaagaaaa aaaaaaaaga ataaataaat aactttttaa aaaaggaaaa aaaaaaaaaa |
| 4321 | a |
| 2. COL3A1 human DNA, Collagen Type III Alpha 1 Chain | |
| (SEQ ID NO: 2) | |
| 1 | ccacgcgtcc ggacgggccc ggtgctgaag ggcagggaac aacttgatgg tgctactttg |
| 61 | aactgctttt cttttctcct ttttgcacaa agagtctcat gtctgatatt tagacatgat |
| 121 | gagctttgtg caaaagggga gctggctact tctcgctctg cttcatccca ctattatttt |
| 181 | ggcacaacag gaagctgttg aaggaggatg ttcccatctt ggtcagtcct atgcggatag |
| 241 | agatgtctgg aagccagaac catgccaaat atgtgtctgt gactcaggat ccgttctctg |
| 301 | cgatgacata atatgtgacg atcaagaatt agactgcccc aacccagaaa ttccatttgg |
| 361 | agaatgttgt gcagtttgcc cacagcctcc aactgctcct actcgccctc ctaatggtca |
| 421 | aggacctcaa ggccccaagg gagatccagg ccctcctggt attcctggga gaaatggtga |
| 481 | ccctggtatt ccaggacaac cagggtcccc tggttctcct ggcccccctg gaatctgtga |
| 541 | atcatgccct actggtcctc agaactattc tccccagtat gattcatatg atgtcaagtc |
| 601 | tggagtagca gtaggaggac tcgcaggcta tcctggacca gctggccccc caggccctcc |
| 661 | cggtccccct ggtacatctg gtcatcctgg ttcccctgga tctccaggat accaaggacc |
| 721 | ccctggtgaa cctgggcaag ctggtccttc aggccctcca ggacctcctg gtgctatagg |
| 781 | tccatctggt cctgctggaa aagatggaga atcaggtaga cccggacgac ctggagagcg |
| 841 | aggattgcct ggacctccag gtatcaaagg tccagctggg atacctggat tccctggtat |
| 901 | gaaaggacac agaggcttcg atggacgaaa tggagaaaag ggtgaaacag gtgctcctgg |
| 961 | attaaagggt gaaaatggtc ttccaggcga aaatggagct cctggaccca tgggtccaag |
| 1021 | aggggctcct ggtgagcgag gacggccagg acttcctggg gctgcaggtg ctcggggtaa |
| 1081 | tgacggtgct cgaggcagtg atggtcaacc aggccctcct ggtcctcctg gaactgccgg |
| 1141 | attccctgga tcccctggtg ctaagggtga agttggacct gcagggtctc ctggttcaaa |
| 1201 | tggtgcccct ggacaaagag gagaacctgg acctcaggga cacgctggtg ctcaaggtcc |
| 1261 | tcctggccct cctgggatta atggtagtcc tggtggtaaa ggcgaaatgg gtcccgctgg |
| 1321 | cattcctgga gctcctggac tgatgggagc ccggggtcct ccaggaccag ccggtgctaa |
| 1381 | tggtgctcct ggactgcgag gtggtgcagg tgagcctggt aagaatggtg ccaaaggaga |
| 1441 | gcccggacca cgtggtgaac gcggtgaggc tggtattcca ggtgttccag gagctaaagg |
| 1501 | cgaagatggc aaggatggat cacctggaga acctggtgca aatgggcttc caggagctgc |
| 1561 | aggagaaagg ggtgcccctg ggttccgagg acctgctgga ccaaatggca tcccaggaga |
| 1621 | aaagggtcct gctggagagc gtggtgctcc aggccctgca gggcccagag gagctgctgg |
| 1681 | agaacctggc agagatggcg tccctggagg tccaggaatg aggggcatgc ccggaagtcc |
| 1741 | aggaggacca ggaagtgatg ggaaaccagg gcctcccgga agtcaaggag aaagtggtcg |
| 1801 | accaggtcct cctgggccat ctggtccccg aggtcagcct ggtgtcatgg gcttccccgg |
| 1861 | tcctaaagga aatgatggtg ctcctggtaa gaatggagaa cgaggtggcc ctggaggacc |
| 1921 | tggccctcag ggtcctcctg gaaagaatgg tgaaactgga cctcagggac ccccagggcc |
| 1981 | tactgggcct ggtggtgaca aaggagacac aggaccccct ggtccacaag gattacaagg |
| 2041 | cttgcctggt acaggtggtc ctccaggaga aaatggaaaa cctggggaac caggtccaaa |
| 2101 | gggtgatgcc ggtgcacctg gagctccagg aggcaagggt gatgctggtg cccctggtga |
| 2161 | acgtggacct cctggattgg caggggcccc aggacttaga ggtggagctg gtccccctgg |
| 2221 | tcccgaagga ggaaagggtg ctgctggtcc tcctgggcca cctggtgctg ctggtactcc |
| 2281 | tggtctgcaa ggaatgcctg gagaaagagg aggtcttgga agtcctggtc caaagggtga |
| 2341 | caagggtgaa ccaggcggtc caggtgctga tggtgtccca gggaaagatg gcccaagggg |
| 2401 | tcctactggt cctattggtc ctcctggccc agctggccag cctggagata agggtgaagg |
| 2461 | tggtgccccc ggacttccag gtatagctgg acctcgtggt agccctggtg agagaggtga |
| 2521 | aactggccct ccaggacctg ctggtttccc tggtgctcct ggacagaatg gtgaacctgg |
| 2581 | tggtaaagga gaaagagggg ctccgggtga gaaaggtgaa ggaggccctc ctggagttgc |
| 2641 | aggacctcct ggcaaagatg gaaccagtgg acatccaggt cccattggac caccagggcc |
| 2701 | tcgaggtaac agaggtgaaa gaggatctga gggctcccca ggccacccag ggcaaccagg |
| 2761 | ccctcctgga cctcctggtg cccctggtcc ttgctgtggt ggtgttggag ccgctgccat |
| 2821 | tgctgggatt ggaggtgaaa aagctggcgg ttttgccccg tattatggag atgaaccaat |
| 2881 | ggatttcaaa atcaacaccg atgagattat gacttcactc aagtctgtta atggacaaat |
| 2941 | agaaagcctc attagtcctg atggttctcg taaaaacccc gctagaaact gcagagacct |
| 3001 | gaaattctgc catcctgaac tcaagagtgg agaatactgg gttgacccta accaaggatg |
| 3061 | caaattggat gctatcaagg tattctgtaa tatggaaact ggggaaacat gcataagtgc |
| 3121 | caatcctttg aatgttccac ggaaacactg gtggacagat tctagtgctg agaagaaaca |
| 3181 | cgtttggttt ggagagtcca tggatggtgg ttttcagttt agctacggca atcctgaact |
| 3241 | tcctgaagat gtccttgatg tgcagctggc attccttcga cttctctcca gccgagcttc |
| 3301 | ccagaacatc acatatcact gcaaaaatag cattgcatac atggatcagg ccagtggaaa |
| 3361 | tgtaaagaag gccctgaagc tgatggggtc aaatgaaggt gaattcaagg ctgaaggaaa |
| 3421 | tagcaaattc acctacacag ttctggagga tggttgcacg aaacacactg gggaatggag |
| 3481 | caaaacagtc tttgaatatc gaacacgcaa ggctgtgaga ctacctattg tagatattgc |
| 3541 | accctatgac attggtggtc ctgatcaaga atttggtgtg gacgttggcc ctgtttgctt |
| 3601 | tttataaacc aaactctatc tgaaatccca acaaaaaaaa tttaactcca tatgtgttcc |
| 3661 | tcttgttcta atcttgtcaa ccagtgcaag tgaccgacaa aattccagtt atttatttcc |
| 3721 | aaaatgtttg gaaacagtat aatttgacaa agaaaaatga tacttctctt tttttgctgt |
| 3781 | tccaccaaat acaattcaaa tgctttttgt tttatttttt taccaattcc aatttcaaaa |
| 3841 | tgtctcaatg gtgctataat aaataaactt caacactctt tatgataaaa aaaaaaaaaa |
| 3901 | aa |
| 3. TIMP1 human DNA, TIMP Metallopeptidase Inhibitor 1 | |
| (SEQ ID NO: 3) | |
| 1 | gtaatgcatc caggaagcct ggaggcctgt ggtttccgca cccgctgcca cccccgcccc |
| 61 | tagcgtggac atttatcctc tagcgctcag gccctgccgc catcgccgca gatccagcgc |
| 121 | ccagagagac accagagaac ccaccatggc cccctttgag cccctggctt ctggcatcct |
| 181 | gttgttgctg tggctgatag cccccagcag ggcctgcacc tgtgtcccac cccacccaca |
| 241 | gacggccttc tgcaattccg acctcgtcat cagggccaag ttcgtgggga caccagaagt |
| 301 | caaccagacc accttatacc agcgttatga gatcaagatg accaagatgt ataaagggtt |
| 361 | ccaagcctta ggggatgccg ctgacatccg gttcgtctac acccccgcca tggagagtgt |
| 421 | ctgcggatac ttccacaggt cccacaaccg cagcgaggag tttctcattg ctggaaaact |
| 481 | gcaggatgga ctcttgcaca tcactacctg cagtttcgtg gctccctgga acagcctgag |
| 541 | cttagctcag cgccggggct tcaccaagac ctacactgtt ggctgtgagg aatgcacagt |
| 601 | gtttccctgt ttatccatcc cctgcaaact gcagagtggc actcattgct tgtggacgga |
| 661 | ccagctcctc caaggctctg aaaagggctt ccagtcccgt caccttgcct gcctgcctcg |
| 721 | ggagccaggg ctgtgcacct ggcagtccct gcggtcccag atagcctgaa tcctgcccgg |
| 781 | agtggaagct gaagcctgca cagtgtccac cctgttccca ctcccatctt tcttccggac |
| 841 | aatgaaataa agagttacca cccagcaaaa aaaaaaaaaa a |
| 4. TIMP2 human DNA, TIMP Metallopeptidase Inhibitor 2 | |
| (SEQ ID NO: 4) | |
| 1 | agcaaacaca tccgtagaag gcagcgcggc cgccgagagc cgcagcgccg ctcgcccgcc |
| 61 | gccccccacc ccgccgcccc gcccggcgaa ttgcgccccg cgcccctccc ctcgcgcccc |
| 121 | cgagacaaag aggagagaaa gtttgcgcgg ccgagcgggg caggtgagga gggtgagccg |
| 181 | cgcgggaggg gcccgcctcg gccccggctc agcccccgcc cgcgccccca gcccgccgcc |
| 241 | gcgagcagcg cccggacccc ccagcggcgg cccccgcccg cccagccccc cggcccgcca |
| 301 | tgggcgccgc ggcccgcacc ctgcggctgg cgctcggcct cctgctgctg gcgacgctgc |
| 361 | ttcgcccggc cgacgcctgc agctgctccc cggtgcaccc gcaacaggcg ttttgcaatg |
| 421 | cagatgtagt gatcagggcc aaagcggtca gtgagaagga agtggactct ggaaacgaca |
| 481 | tttatggcaa ccctatcaag aggatccagt atgagatcaa gcagataaag atgttcaaag |
| 541 | ggcctgagaa ggatatagag tttatctaca cggccccctc ctcggcagtg tgtggggtct |
| 601 | cgctggacgt tggaggaaag aaggaatatc tcattgcagg aaaggccgag ggggacggca |
| 661 | agatgcacat caccctctgt gacttcatcg tgccctggga caccctgagc accacccaga |
| 721 | agaagagcct gaaccacagg taccagatgg gctgcgagtg caagatcacg cgctgcccca |
| 781 | tgatcccgtg ctacatctcc tccccggacg agtgcctctg gatggactgg gtcacagaga |
| 841 | agaacatcaa cgggcaccag gccaagttct tcgcctgcat caagagaagt gacggctcct |
| 901 | gtgcgtggta ccgcggcgcg gcgcccccca agcaggagtt tctcgacatc gaggacccat |
| 961 | aagcaggcct ccaacgcccc tgtggccaac tgcaaaaaaa gcctccaagg gtttcgactg |
| 1021 | gtccagctct gacatccctt cctggaaaca gcatgaataa aacactcatc ccatgggtcc |
| 1081 | aaattaatat gattctgctc cccccttctc cttttagaca tggttgtggg tctggaggga |
| 1141 | gacgtgggtc caaggtcctc atcccatcct ccctctgcca ggcactatgt gtctggggct |
| 1201 | tcgatccttg ggtgcaggca gggctgggac acgcggcttc cctcccagtc cctgccttgg |
| 1261 | caccgtcaca gatgccaagc aggcagcact tagggatctc ccagctgggt tagggcaggg |
| 1321 | cctggaaatg tgcattttgc agaaactttt gagggtcgtt gcaagactgt gtagcaggcc |
| 1381 | taccaggtcc ctttcatctt gagagggaca tggcccttgt tttctgcagc ttccacgcct |
| 1441 | ctgcactccc tgcccctggc aagtgctccc atcgccccgg tgcccaccat gagctcccag |
| 1501 | cacctgactc cccccacatc caagggcagc ctggaaccag tggctagttc ttgaaggagc |
| 1561 | cccatcaatc ctattaatcc tcagaattcc agtgggagcc tccctctgag ccttgtagaa |
| 1621 | atgggagcga gaaaccccag ctgagctgcg ttccagcctc agctgagtct ttttggtctg |
| 1681 | cacccacccc cccacccccc ccccgcccac atgctcccca gcttgcagga ggaatcggtg |
| 1741 | aggtcctgtc ctgaggctgc tgtccggggc cggtggctgc cctcaaggtc ccttccctag |
| 1801 | ctgctgcggt tgccattgct tcttgcctgt tctggcatca ggcacctgga ttgagttgca |
| 1861 | cagctttgct ttatccgggc ttgtgtgcag ggcccggctg ggctccccat ctgcacatcc |
| 1921 | tgaggacaga aaaagctggg tcttgctgtg ccctcccagg cttagtgttc cctccctcaa |
| 1981 | agactgacag ccatcgttct gcacggggtt ttctgcatgt gacgccagct aagcatagta |
| 2041 | agaagtccag cctaggaagg gaaggatttt ggaggtaggt ggctttggtg acacactcac |
| 2101 | ttctttctca gcctccagga cactatggcc tgttttaaga gacatcttat ttttctaaag |
| 2161 | gtgaattctc agatgatagg tgaacctgag ttgcagatat accaacttct gcttgtattt |
| 2221 | cttaaatgac aaagattacc tagctaagaa acttcctagg gaactaggga acctatgtgt |
| 2281 | tccctcagtg tggtttcctg aagccagtga tatgggggtt aggataggaa gaactttctc |
| 2341 | ggtaatgata aggagaatct cttgtttcct cccacctgtg ttgtaaagat aaactgacga |
| 2401 | tatacaggca cattatgtaa acatacacac gcaatgaaac cgaagcttgg cggcctgggc |
| 2461 | gtggtcttgc aaaatgcttc caaagccacc ttagcctgtt ctattcagcg gcaaccccaa |
| 2521 | agcacctgtt aagactcctg acccccaagt ggcatgcagc ccccatgccc accgggacct |
| 2581 | ggtcagcaca gatcttgatg acttcccttt ctagggcaga ctgggagggt atccaggaat |
| 2641 | cggcccctgc cccacgggcg ttttcatgct gtacagtgac ctaaagttgg taagatgtca |
| 2701 | taatggacca gtccatgtga tttcagtata tacaactcca ccagacccct ccaacccata |
| 2761 | taacacccca cccctgttcg cttcctgtat ggtgatatca tatgtaacat ttactcctgt |
| 2821 | ttctgctgat tgttttttta atgttttggt ttgtttttga catcagctgt aatcattcct |
| 2881 | gtgctgtgtt ttttattacc cttggtaggt attagacttg cactttttta aaaaaaggtt |
| 2941 | tctgcatcgt ggaagcattt gacccagagt ggaacgcgtg gcctatgcag gtggattcct |
| 3001 | tcaggtcttt cctttggttc tttgagcatc tttgctttca ttcgtctccc gtctttggtt |
| 3061 | ctccagttca aattattgca aagtaaagga tctttgagta ggttcggtct gaaaggtgtg |
| 3121 | gcctttatat ttgatccaca cacgttggtc ttttaaccgt gctgagcaga aaacaaaaca |
| 3181 | ggttaagaag agccgggtgg cagctgacag aggaagccgc tcaaatacct tcacaataaa |
| 3241 | tagtggcaat atatatatag tttaagaagg ctctccattt ggcatcgttt aatttatatg |
| 3301 | ttatgttcta agcacagctc tcttctccta ttttcatcct gcaagcaact caaaatattt |
| 3361 | aaaataaagt ttacattgta gttattttca aatctttgct tgataagtat taagaaatat |
| 3421 | tggacttgct gccgtaattt aaagctctgt tgattttgtt tccgtttgga tttttggggg |
| 3481 | aggggagcac tgtgtttatg ctggaatatg aagtctgaga ccttccggtg ctgggaacac |
| 3541 | acaagagttg ttgaaagttg acaagcagac tgcgcatgtc tctgatgctt tgtatcattc |
| 3601 | ttgagcaatc gctcggtccg tggacaataa acagtattat caaagagaaa aaaaaaaaaa |
| 3661 | a |
| 5. SMAD2 human DNA, SMAD Family Member 2 | |
| (SEQ ID NO: 5) | |
| 1 | gcgcccgggc cgccggccgg gcccgggcct gggggcgggg cgggaagacg gcggccggga |
| 61 | gtgttttcag ttccgcctcc aatcgcccat tcccctcttc ccctcccagc cccctccatc |
| 121 | ccatcggaag aggaaggaac aaaaggtccc ggaccccccg gatctgacgg ggcgggacct |
| 181 | ggcgccacct tgcaggttcg atacaagagg ctgttttcct agcgtggctt gctgcctttg |
| 241 | gtaagaacat gtcgtccatc ttgccattca cgccgccagt tgtgaagaga ctgctgggat |
| 301 | ggaagaagtc agctggtggg tctggaggag caggcggagg agagcagaat gggcaggaag |
| 361 | aaaagtggtg tgagaaagca gtgaaaagtc tggtgaagaa gctaaagaaa acaggacgat |
| 421 | tagatgagct tgagaaagcc atcaccactc aaaactgtaa tactaaatgt gttaccatac |
| 481 | caagcacttg ctctgaaatt tggggactga gtacaccaaa tacgatagat cagtgggata |
| 541 | caacaggcct ttacagcttc tctgaacaaa ccaggtctct tgatggtcgt ctccaggtat |
| 601 | cccatcgaaa aggattgcca catgttatat attgccgatt atggcgctgg cctgatcttc |
| 661 | acagtcatca tgaactcaag gcaattgaaa actgcgaata tgcttttaat cttaaaaagg |
| 721 | atgaagtatg tgtaaaccct taccactatc agagagttga gacaccagtt ttgcctccag |
| 781 | tattagtgcc ccgacacacc gagatcctaa cagaacttcc gcctctggat gactatactc |
| 841 | actccattcc agaaaacact aacttcccag caggaattga gccacagagt aattatattc |
| 901 | cagaaacgcc acctcctgga tatatcagtg aagatggaga aacaagtgac caacagttga |
| 961 | atcaaagtat ggacacaggc tctccagcag aactatctcc tactactctt tcccctgtta |
| 1021 | atcatagctt ggatttacag ccagttactt actcagaacc tgcattttgg tgttcgatag |
| 1081 | catattatga attaaatcag agggttggag aaaccttcca tgcatcacag ccctcactca |
| 1141 | ctgtagatgg ctttacagac ccatcaaatt cagagaggtt ctgcttaggt ttactctcca |
| 1201 | atgttaaccg aaatgccacg gtagaaatga caagaaggca tataggaaga ggagtgcgct |
| 1261 | tatactacat aggtggggaa gtttttgctg agtgcctaag tgatagtgca atctttgtgc |
| 1321 | agagccccaa ttgtaatcag agatatggct ggcaccctgc aacagtgtgt aaaattccac |
| 1381 | caggctgtaa tctgaagatc ttcaacaacc aggaatttgc tgctcttctg gctcagtctg |
| 1441 | ttaatcaggg ttttgaagcc gtctatcagc taactagaat gtgcaccata agaatgagtt |
| 1501 | ttgtgaaagg gtggggagca gaataccgaa ggcagacggt aacaagtact ccttgctgga |
| 1561 | ttgaacttca tctgaatgga cctctacagt ggttggacaa agtattaact cagatgggat |
| 1621 | ccccttcagt gcgttgctca agcatgtcat aaagcttcac caatcaagtc ccatgaaaag |
| 1681 | acttaatgta acaactcttc tgtcatagca ttgtgtgtgg tccctatgga ctgtttacta |
| 1741 | tccaaaagtt caagagagaa aacagcactt gaggtctcat caattaaagc accttgtgga |
| 1801 | atctgtttcc tatatttgaa tattagatgg gaaaattagt gtctagaaat actctcccat |
| 1861 | taaagaggaa gagaagattt taaagactta atgatgtctt attgggcata aaactgagtg |
| 1921 | tcccaaaggt ttattaataa cagtagtagt tatgtgtaca ggtaatgtat catgatccag |
| 1981 | tatcacagta ttgtgctgtt tatatacatt tttagtttgc atagatgagg tgtgtgtgtg |
| 2041 | cgctgcttct tgatctaggc aaacctttat aaagttgcag tacctaatct gttattccca |
| 2101 | cttctctgtt atttttgtgt gtctttttta atatataata tatatcaaga ttttcaaatt |
| 2161 | atttagaagc agattttcct gtagaaaaac taatttttct gccttttacc aaaaataaac |
| 2221 | tcttggggga agaaaaaaaa aaaaaaaaaa aaaaaaaaaa aaaaaaaaaa aaaaaaaaaa |
| 2281 | aaaaa |
| 6. SMAD3 human DNA, SMAD Family Member 3 | |
| (SEQ ID NO: 6) | |
| 1 | gccgtgggag ccgctccggg cgcagggccg cgcgccgagc cccgcaggct gcagcgccgc |
| 61 | ggcccggccc ggcgccccgg caacttcgcc gagagttgag gcgaagtttg ggcgaccgcg |
| 121 | gcaggccccg gccgagctcc cctctgcgcc cccggcgtcc cgtcgagccc agccccgccg |
| 181 | ggggcgctcc tcgccgcccg cgcgccctcc ccagccatgt cgtccatcct gcctttcact |
| 241 | cccccgatcg tgaagcgcct gctgggctgg aagaagggcg agcagaacgg gcaggaggag |
| 301 | aaatggtgcg agaaggcggt caagagcctg gtcaagaaac tcaagaagac ggggcagctg |
| 361 | gacgagctgg agaaggccat caccacgcag aacgtcaaca ccaagtgcat caccatcccc |
| 421 | aggtccctgg atggccggtt gcaggtgtcc catcggaagg ggctccctca tgtcatctac |
| 481 | tgccgcctgt ggcgatggcc agacctgcac agccaccacg agctgcgggc catggagctg |
| 541 | tgtgagttcg ccttcaatat gaagaaggac gaggtctgcg tgaatcccta ccactaccag |
| 601 | agagtagaga caccagttct acctcctgtg ttggtgccac gccacacaga gatcccggcc |
| 661 | gagttccccc cactggacga ctacagccat tccatccccg aaaacactaa cttccccgca |
| 721 | ggcatcgagc cccagagcaa tattccagag accccacccc ctggctacct gagtgaagat |
| 781 | ggagaaacca gtgaccacca gatgaaccac agcatggacg caggttctcc aaacctatcc |
| 841 | ccgaatccga tgtccccagc acataataac ttggacctgc agccagttac ctactgcgag |
| 901 | ccggccttct ggtgctccat ctcctactac gagctgaacc agcgcgtcgg ggagacattc |
| 961 | cacgcctcgc agccatccat gactgtggat ggcttcaccg acccctccaa ttcggagcgc |
| 1021 | ttctgcctag ggctgctctc caatgtcaac aggaatgcag cagtggagct gacacggaga |
| 1081 | cacatcggaa gaggcgtgcg gctctactac atcggagggg aggtcttcgc agagtgcctc |
| 1141 | agtgacagcg ctatttttgt ccagtctccc aactgtaacc agcgctatgg ctggcacccg |
| 1201 | gccaccgtct gcaagatccc accaggatgc aacctgaaga tcttcaacaa ccaggagttc |
| 1261 | gctgccctcc tggcccagtc ggtcaaccag ggctttgagg ctgtctacca gttgacccga |
| 1321 | atgtgcacca tccgcatgag cttcgtcaaa ggctggggag cggagtacag gagacagact |
| 1381 | gtgaccagta ccccctgctg gattgagctg cacctgaatg ggcctttgca gtggcttgac |
| 1441 | aaggtcctca cccagatggg ctccccaagc atccgctgtt ccagtgtgtc ttagagacat |
| 1501 | caagtatggt aggggagggc aggcttgggg aaaatggcca tgcaggaggt ggagaaaatt |
| 1561 | ggaactctac tcaacccatt gttgtcaagg aagaagaaat ctttctccct caactgaagg |
| 1621 | ggtgcaccca cctgttttct gaaacacacg agcaaaccca gaggtggatg ttatgaacag |
| 1681 | ctgtgtctgc caaacacatt taccctttgg ccccactttg aagggcaaga aatggcgtct |
| 1741 | gctctggtgg cttaagtgag cagaacaggt agtattacac caccggcccc ctccccccag |
| 1801 | actctttttt tgagtgacag ctttctggga tgtcacagtc caaccagaaa cacccctctg |
| 1861 | tctaggactg cagtgtggag ttcaccttgg aagggcgttc taggtaggaa gagcccgcag |
| 1921 | ggccatgcag acctcatgcc cagctctctg acgcttgtga cagtgcctct tccagtgaac |
| 1981 | attcccagcc cagccccgcc ccgccccgcc ccaccactcc agcagacctt gccccttgtg |
| 2041 | agctggatag acttgggatg gggagggagg gagttttgtc tgtctccctc ccctctcaga |
| 2101 | acatactgat tgggaggtgc gtgttcagca gaacctgcac acaggacagc gggaaaaatc |
| 2161 | gatgagcgcc acctctttaa aaactcactt acgtttgtcc tttttcactt tgaaaagttg |
| 2221 | gaaggatctg ctgaggccca gtgcatatgc aatgtatagt gtctattatc acattaatct |
| 2281 | caaagagatt cgaatgacgg taagtgttct catgaagcag gaggcccttg tcgtgggatg |
| 2341 | gcatttggtc tcaggcagca ccacactggg tgcgtctcca gtcatctgta agagcttgct |
| 2401 | ccagattctg atgcatacgg ctatattggt ttatgtagtc agttgcattc attaaatcaa |
| 2461 | ctttatcata aaaaaaaaaa aaaaa |
| 7. CTGF human DNA, Connective Tissue Growth Factor | |
| (SEQ ID NO: 7) | |
| 1 | gctgagagga gacagccagt gcgactccac cctccagctc gacggcagcc gccccggccg |
| 61 | acagccccga gacgacagcc cggcgcgtcc cggtccccac ctccgaccac cgccagcgct |
| 121 | ccaggccccg ccgctccccg ctcgccgcca ccgcgccctc cgctccgccc gcagtgccaa |
| 181 | ccatgaccgc cgccagtatg ggccccgtcc gcgtcgcctt cgtggtcctc ctcgccctct |
| 241 | gcagccggcc ggccgtcggc cagaactgca gcgggccgtg ccggtgcccg gacgagccgg |
| 301 | cgccgcgctg cccggcgggc gtgagcctcg tgctggacgg ctgcggctgc tgccgcgtct |
| 361 | gcgccaagca gctgggcgag ctgtgcaccg agcgcgaccc ctgcgacccg cacaagggcc |
| 421 | tcttctgtga cttcggctcc ccggccaacc gcaagatcgg cgtgtgcacc gccaaagatg |
| 481 | gtgctccctg catcttcggt ggtacggtgt accgcagcgg agagtccttc cagagcagct |
| 541 | gcaagtacca gtgcacgtgc ctggacgggg cggtgggctg catgcccctg tgcagcatgg |
| 601 | acgttcgtct gcccagccct gactgcccct tcccgaggag ggtcaagctg cccgggaaat |
| 661 | gctgcgagga gtgggtgtgt gacgagccca aggaccaaac cgtggttggg cctgccctcg |
| 721 | cggcttaccg actggaagac acgtttggcc cagacccaac tatgattaga gccaactgcc |
| 781 | tggtccagac cacagagtgg agcgcctgtt ccaagacctg tgggatgggc atctccaccc |
| 841 | gggttaccaa tgacaacgcc tcctgcaggc tagagaagca gagccgcctg tgcatggtca |
| 901 | ggccttgcga agctgacctg gaagagaaca ttaagaaggg caaaaagtgc atccgtactc |
| 961 | ccaaaatctc caagcctatc aagtttgagc tttctggctg caccagcatg aagacatacc |
| 1021 | gagctaaatt ctgtggagta tgtaccgacg gccgatgctg caccccccac agaaccacca |
| 1081 | ccctgccggt ggagttcaag tgccctgacg gcgaggtcat gaagaagaac atgatgttca |
| 1141 | tcaagacctg tgcctgccat tacaactgtc ccggagacaa tgacatcttt gaatcgctgt |
| 1201 | actacaggaa gatgtacgga gacatggcat gaagccagag agtgagagac attaactcat |
| 1261 | tagactggaa cttgaactga ttcacatctc atttttccgt aaaaatgatt tcagtagcac |
| 1321 | aagttattta aatctgtttt tctaactggg ggaaaagatt cccacccaat tcaaaacatt |
| 1381 | gtgccatgtc aaacaaatag tctatcaacc ccagacactg gtttgaagaa tgttaagact |
| 1441 | tgacagtgga actacattag tacacagcac cagaatgtat attaaggtgt ggctttagga |
| 1501 | gcagtgggag ggtaccagca gaaaggttag tatcatcaga tagcatctta tacgagtaat |
| 1561 | atgcctgcta tttgaagtgt aattgagaag gaaaatttta gcgtgctcac tgacctgcct |
| 1621 | gtagccccag tgacagctag gatgtgcatt ctccagccat caagagactg agtcaagttg |
| 1681 | ttccttaagt cagaacagca gactcagctc tgacattctg attcgaatga cactgttcag |
| 1741 | gaatcggaat cctgtcgatt agactggaca gcttgtggca agtgaatttg cctgtaacaa |
| 1801 | gccagatttt ttaaaattta tattgtaaat attgtgtgtg tgtgtgtgtg tgtatatata |
| 1861 | tatatatgta cagttatcta agttaattta aagttgtttg tgccttttta tttttgtttt |
| 1921 | taatgctttg atatttcaat gttagcctca atttctgaac accataggta gaatgtaaag |
| 1981 | cttgtctgat cgttcaaagc atgaaatgga tacttatatg gaaattctgc tcagatagaa |
| 2041 | tgacagtccg tcaaaacaga ttgtttgcaa aggggaggca tcagtgtcct tggcaggctg |
| 2101 | atttctaggt aggaaatgtg gtagcctcac ttttaatgaa caaatggcct ttattaaaaa |
| 2161 | ctgagtgact ctatatagct gatcagtttt ttcacctgga agcatttgtt tctactttga |
| 2221 | tatgactgtt tttcggacag tttatttgtt gagagtgtga ccaaaagtta catgtttgca |
| 2281 | cctttctagt tgaaaataaa gtgtatattt tttctataaa aaaaaaaaaa aaaa |
| 8. TGF-b1 human DNA, Transforming Growth Factor Beta 1 | |
| (SEQ ID NO: 8) | |
| 1 | cccagacctc gggcgcaccc cctgcacgcc gccttcatcc ccggcctgtc tcctgagccc |
| 61 | ccgcgcatcc tagacccttt ctcctccagg agacggatct ctctccgacc tgccacagat |
| 121 | cccctattca agaccaccca ccttctggta ccagatcgcg cccatctagg ttatttccgt |
| 181 | gggatactga gacacccccg gtccaagcct cccctccacc actgcgccct tctccctgag |
| 241 | gacctcagct ttccctcgag gccctcctac cttttgccgg gagaccccca gcccctgcag |
| 301 | gggcggggcc tccccaccac accagccctg ttcgcgctct cggcagtgcc ggggggcgcc |
| 361 | gcctccccca tgccgccctc cgggctgcgg ctgctgctgc tgctgctacc gctgctgtgg |
| 421 | ctactggtgc tgacgcctgg ccggccggcc gcgggactat ccacctgcaa gactatcgac |
| 481 | atggagctgg tgaagcggaa gcgcatcgag gccatccgcg gccagatcct gtccaagctg |
| 541 | cggctcgcca gccccccgag ccagggggag gtgccgcccg gcccgctgcc cgaggccgtg |
| 601 | ctcgccctgt acaacagcac ccgcgaccgg gtggccgggg agagtgcaga accggagccc |
| 661 | gagcctgagg ccgactacta cgccaaggag gtcacccgcg tgctaatggt ggaaacccac |
| 721 | aacgaaatct atgacaagtt caagcagagt acacacagca tatatatgtt cttcaacaca |
| 781 | tcagagctcc gagaagcggt acctgaaccc gtgttgctct cccgggcaga gctgcgtctg |
| 841 | ctgaggctca agttaaaagt ggagcagcac gtggagctgt accagaaata cagcaacaat |
| 901 | tcctggcgat acctcagcaa ccggctgctg gcacccagcg actcgccaga gtggttatct |
| 961 | tttgatgtca ccggagttgt gcggcagtgg ttgagccgtg gaggggaaat tgagggcttt |
| 1021 | cgccttagcg cccactgctc ctgtgacagc agggataaca cactgcaagt ggacatcaac |
| 1081 | gggttcacta ccggccgccg aggtgacctg gccaccattc atggcatgaa ccggcctttc |
| 1141 | ctgcttctca tggccacccc gctggagagg gcccagcatc tgcaaagctc ccggcaccgc |
| 1201 | cgagccctgg acaccaacta ttgcttcagc tccacggaga agaactgctg cgtgcggcag |
| 1261 | ctgtacattg acttccgcaa ggacctcggc tggaagtgga tccacgagcc caagggctac |
| 1321 | catgccaact tctgcctcgg gccctgcccc tacatttgga gcctggacac gcagtacagc |
| 1381 | aaggtcctgg ccctgtacaa ccagcataac ccgggcgcct cggcggcgcc gtgctgcgtg |
| 1441 | ccgcaggcgc tggagccgct gcccatcgtg tactacgtgg gccgcaagcc caaggtggag |
| 1501 | cagctgtcca acatgatcgt gcgctcctgc aagtgcagct gaggtcccgc cccgccccgc |
| 1561 | cccgccccgg caggcccggc cccaccccgc cccgcccccg ctgccttgcc catgggggct |
| 1621 | gtatttaagg acacccgtgc cccaagccca cctggggccc cattaaagat ggagagagga |
| 1681 | aaaaaaaaaa aaaaaaaaaa aaaaaaaaaa aaaaaaaaaa aaaaaaaaaa aaaaaaaaaa |
| 1741 | aaaaaa |
| 9. KRT6A human DNA, Keratin 6A | |
| (SEQ ID NO: 9) | |
| 1 | ctctcctcca gcctctcaca ctctcctcag ctctctcatc tcctggaacc atggccagca |
| 61 | catccaccac catcaggagc cacagcagca gccgccgggg tttcagtgcc agctcagcca |
| 121 | ggctccctgg ggtcagccgc tctggcttca gcagcgtctc cgtgtcccgc tccaggggca |
| 181 | gtggtggcct gggtggtgca tgtggaggag ctggctttgg cagccgcagt ctgtatggcc |
| 241 | tggggggctc caagaggatc tccattggag ggggcagctg tgccatcagt ggcggctatg |
| 301 | gcagcagagc cggaggcagc tatggctttg gtggcgccgg gagtggattt ggtttcggtg |
| 361 | gtggagccgg cattggcttt ggtctgggtg gtggagccgg ccttgctggt ggctttgggg |
| 421 | gccctggctt ccctgtgtgc ccccctggag gcatccaaga ggtcaccgtc aaccagagtc |
| 481 | tcctgactcc cctcaacctg caaatcgatc ccaccatcca gcgggtgcgg gccgaggagc |
| 541 | gtgaacagat caagaccctc aacaacaagt ttgcctcctt catcgacaag gtgcggttcc |
| 601 | tggagcagca gaacaaggtt ctggaaacaa agtggaccct gctgcaggag cagggcacca |
| 661 | agactgtgag gcagaacctg gagccgttgt tcgagcagta catcaacaac ctcaggaggc |
| 721 | agctggacag cattgtcggg gaacggggcc gcctggactc agagctcaga ggcatgcagg |
| 781 | acctggtgga ggacttcaag aacaaatatg aggatgaaat caacaagcgc acagcagcag |
| 841 | agaatgaatt tgtgactctg aagaaggacg tggatgctgc ctacatgaac aaggttgaac |
| 901 | tgcaagccaa ggcagacact ctcacagacg agatcaactt cctgagagcc ttgtatgatg |
| 961 | cagagctgtc ccagatgcag acccacatct cagacacatc tgtggtgctg tccatggaca |
| 1021 | acaaccgcaa cctggacctg gacagcatca tcgctgaggt caaggcccaa tatgaggaga |
| 1081 | ttgctcagag aagccgggct gaggctgagt cctggtacca gaccaagtac gaggagctgc |
| 1141 | aggtcacagc aggcagacat ggggacgacc tgcgcaacac caagcaggag attgctgaga |
| 1201 | tcaaccgcat gatccagagg ctgagatctg agatcgacca cgtcaagaag cagtgcgcca |
| 1261 | acctgcaggc cgccattgct gatgctgagc agcgtgggga gatggccctc aaggatgcca |
| 1321 | agaacaagct ggaagggctg gaggatgccc tgcagaaggc caagcaggac ctggcccggc |
| 1381 | tgctgaagga gtaccaggag ctgatgaatg tcaagctggc cctggacgtg gagatcgcca |
| 1441 | cctaccgcaa gctgctggag ggtgaggagt gcaggctgaa tggcgaaggc gttggacaag |
| 1501 | tcaacatctc tgtggtgcag tccaccgtct ccagtggcta tggcggtgcc agtggtgtcg |
| 1561 | gcagtggctt aggcctgggt ggaggaagca gctactccta tggcagtggt cttggcgttg |
| 1621 | gaggtggctt cagttccagc agtggcagag ccattggggg tggcctcagc tctgttggag |
| 1681 | gcggcagttc caccatcaag tacaccacca cctcctcctc cagcaggaag agctataagc |
| 1741 | actaaagtgc gtctgctagc tctcggtccc acagtcctca ggcccctctc tggctgcaga |
| 1801 | gccctctcct caggttgcct gtcctctcct ggcctccagt ctcccctgct gtcccaggta |
| 1861 | gagctgggga tgaatgctta gtgccttcac ttcttctctc tctctctata ccatctgagc |
| 1921 | acccattgct caccatcaga tcaacctctg attttacatc atgatgtaat caccactgga |
| 1981 | gcttcacttt gttactaaat tattaatttc ttgcctccag tgttctatct ctgaggctga |
| 2041 | gcattataag aaaatgacct ctgctccttt tcattgcaga aaattgccag gggcttattt |
| 2101 | cagaacaact tccacttact ttccactggc tctcaaactc tctaacttat aagtgttgtg |
| 2161 | aacccccacc caggcagtat ccatgaaagc acaagtgact agtcctatga tgtacaaagc |
| 2221 | ctgtatctct gtgatgattt ctgtgctctt cgctctttgc aattgctaaa taaagcagat |
| 2281 | ttataataca ataaaaaaaa aaaaaaaaaa aaaaaaaaaa aaaaaaaaaa aaaaaaaaaa |
| 2341 | aaaaa |
| 10. NOTCH1 (cytoplasmic domain) human DNA, Notch 1 | |
| (SEQ ID NO: 10) | |
| tcccgcaagcgccggcggcagcatggccagctctggttccctgagggtttcaaagtgtcagaggccagca | |
| agaagaagcggagagagcccctcggcgaggactcagtcggcctcaagcccctgaagaatgcctcagatgg | |
| tgctctgatggacgacaatcagaacgagtggggagacgaagacctggagaccaagaagttccggtttgag | |
| gagccagtagttctccctgacctgagtgatcagactgaccacaggcagtggacccagcagcacctggacg | |
| ctgctgacctgcgcatgtctgccatggccccaacaccgcctcagggggaggtggatgctgactgcatgga | |
| tgtcaatgttcgaggaccagatggcttcacacccctcatgattgcctcctgcagtggagggggccttgag | |
| acaggcaacagtgaagaagaagaagatgcacctgctgtcatctctgacttcatctaccagggcgccagct | |
| tgcacaaccagacagaccgcaccggggagaccgccttgcacttggctgcccgatactctcgttcagatgc | |
| tgcaaagcgcttgctggaggccagtgcagatgccaacatccaggacaacatgggccgtactccgttacat | |
| gcagcagtttctgcagatgctcagggtgtcttccagatcctgctccggaacagggccacagatctggatg | |
| cccgaatgcatgatggcacaactccactgatcctggctgcgcgcctggccgtggagggcatgctggagga | |
| cctcatcaactcacatgctgacgtcaatgccgtggatgacctaggcaagtcggctttgcattgggcggcc | |
| gcggtgaacaatgtggatgctgctgttgtgctcctgaagaacggagccaacaaggacatgcagaacaaca | |
| aggaggagactcccctgttcctggccgcccgtgagggcagctatgagactgccaaagtgttgctggacca | |
| ctttgccaaccgggacatcacggatcacatggaccgattgccgcgggacatcgcacaggagcgtatgcac | |
| cacgatatcgtgcggcttttggatgagtacaacctggtgcgcagcccacagctgcatggcactgccctgg | |
| gtggcacacccactctgtctcccacactctgctcgcccaatggctacctgggcaatctcaagtctgccac | |
| acagggcaagaaggcccgcaagcccagcaccaaagggctggcttgtggtagcaaggaagctaaggacctc | |
| aaggcacggaggaagaagtcccaggatggcaagggctgcctgttggacagctcgagcatgctgtcgcctg | |
| tggactccctcgagtcaccccatggctacttgtcagatgtggcctcgccacccctcctcccctccccatt | |
| ccagcagtctccatccatgcctctcagccacctgcctggtatgcctgacactcacctgggcatcagccac | |
| ttgaatgtggcagccaagcctgagatggcagcactggctggaggtagccggttggcctttgagccacccc | |
| cgccacgcctctcccacctgcctgtagcctccagtgccagcacagtgctgagtaccaatggcacgggggc | |
| tatgaatttcaccgtgggtgcaccggcaagcttgaatggccagtgtgagtggcttccccggctccagaat | |
| ggcatggtgcccagccagtacaacccactacggccgggtgtgacgccgggcacactgagcacacaggcag | |
| ctggcctccagcatagcatgatggggccactacacagcagcctctccaccaataccttgtccccgattat | |
| ttaccagggcctgcccaacacacggctggcaacacagcctcacctggtgcagacccagcaggtgcagcca | |
| cagaacttacagctccagcctcagaacctgcagccaccatcacagccacacctcagtgtgagctcggcag | |
| ccaatgggcacctgggccggagcttcttgagtggggagcccagtcaggcagatgtacaaccgctgggccc | |
| cagcagtctgcctgtgcacaccattctgccccaggaaagccaggccctgcccacatcactgccatcctcc | |
| atggtcccacccatgaccactacccagttcctgacccctccttcccagcacagttactcctcctcccctg | |
| tggacaacacccccagccaccagctgcaggtgccagagcaccccttcctcaccccatcccctgagtcccc | |
| tgaccagtggtccagctcctccccgcattccaacatctctgattggtccgagggcatctccagcccgccc | |
| accaccatgccgtcccagatcacccacattccagaggcatttaaataa | |
| 11. TET2 mouse DNA, Tet Methylcytosine Dioxygenase 2 | |
| (SEQ ID NO: 11) | |
| 1 | catcaatgct acttaacatg tgttcatggg caagtcatat ttaggagtat gtgctaccat |
| 61 | aacaattgtg catgtgcaca cacacacact cacatatttc actaatgagt agtttgggca |
| 121 | taaatttgaa agagagcagg gagggttata agtgagggtt tggagggagg aaacggatgg |
| 181 | ggaaatgtgg gacctggcag tgctagattg cttaccttac tacaccgaga agccttttcc |
| 241 | tcagtaatag tgtgctctat ttttggtcat (ccattatgc tctugatata aagtgcaaaa |
| 301 | gtctaaagaa ctttcccatt gacacacatc tgtctgtcag gitgaatttg aacaccaagc |
| 361 | cccagactgc tgtttgggtc tgaaggaagg ccggccattc Icaggagtca ctgcatgttt |
| 421 | ggacttctct gctcattccc acagagacca gcagaacatg ccaaatggca gtacagtggt |
| 481 | ggtcaccctc aatagagaag acaatcgaga agtcggagct aagcctgagg atgagcagtt |
| 541 | ccacgtgctg cctatgtaca tcatcgcccc Igaggatgag tttgggagta cggaaggcca |
| 601 | ggagaagaag atacggatgg ggtccattga ggttctgcag tcatttcgga ggagaagggt |
| 661 | cataaggata ggagagctgc ccaagagttg caagaagaaa gcggagccca agaaagccaa |
| 721 | gaccaagaaa gcagctcgaa agcgttcctc (ctggagaac tgctccagta ggactgagaa |
| 781 | gggaaagtct tcctcacata caaagctgat ggaaaatgca agccatatga aacaaatgac |
| 841 | agcacaaccg cagctttcgg gcccggtcat ccggcagcca ccaacactcc agaggcacct |
| 901 | tcagcaaggg cagaggccac agcagccgca gccacctcag ccgcagccgc agacgacacc |
| 961 | tcagccacag ccacagccac agcatatcat gcccggtaac tctcagtctg ttggttctca |
| 1021 | ttgttctgga tccaccagtg tctacacgag acagcctact cctcacagtc cttatcccag |
| 1081 | ctcagcacac acctcagata tttatggaga taccaaccat gtgaactttt accccacttc |
| 1141 | atctcatgcc tcgggttcat atttgaatcc ttctaattac atgaacccct accttgggct |
| 1201 | tttgaatcag aataaccaat atgcaccttt tccatacaat gggagtgtgc cagtggacaa |
| 1261 | tggttcccct ttcttaggtt cttattcccc ccaggctcag tccagggatc tacatagata |
| 1321 | tccaaaccag gaccatctca ccaatcagaa cttaccaccc atccacaccc ttcaccaaca |
| 1381 | gacgtttggg gacagtccct ctaagtactt aagttatgga aaccaaaata tgcagagaga |
| 1441 | tgccttcact actaactcca ccctaaaacc aaatgtacac cacctagcaa cgttttctcc |
| 1501 | ttaccccacc cccaagatgg atagtcattt catgggagct gcctccagat caccatacag |
| 1561 | ccacccacac actgactaca aaaccagtga gcatcatcta ccctctcaca cgatctacag |
| 1621 | ctacacggca gcagcttcgg ggagcagttc cagccacgcc ttccacaaca aggagaatga |
| 1681 | caacatagcc aatgggctct caagagtgct tccagggttt aatcatgata gaactgcttc |
| 1741 | tgcccaagaa ctattataca gtctgactgg cagcagtcag gagaagcagc ctgaggtgtc |
| 1801 | aggccaggat gcagctgctg (gcaggaaat tgagtattgg icagatagtg agcacaactt |
| 1861 | tcaggatcct tgcattggag gggtggctat agctccaact catgggtcaa ItcttatCga |
| 1921 | gtgtgcaaag tgtgaggttc atgccacaac caaagtaaac gatcccgacc ggaatcaccc |
| 1981 | caccaggatc tcacttgtac tgtataggca taagaatttg tttctaccaa aacattgttt |
| 2041 | ggctctctgg gaagccaaaa tggctgaaaa ggcccggaaa gaggaagagt gcggaaagaa |
| 2101 | tggatcagac cacgtgtctc agaaaaatca tggcaaacag gaaaagcgtg agcccacagg |
| 2161 | gccacaggaa cccagttacc tgcgtttcat ccagtctctt gctgagaaca cagggtctgt |
| 2221 | gactacggat tctaccgtga ctacatcacc atatgctttc actcaggtca cagggcctta |
| 2281 | caacacattt gtatgacgct ggccattagg ccagaccacc aaggacgacc tgtgagcagt |
| 2341 | atgtctttca tggcatgggc cgtagggaca ggtcacagca tctgtgacaa atgcagtgtg |
| 2401 | tgtttgtgtg tatgtttatt gggggggggc tgtcagctca ccagcaaaat agtttatttt |
| 2461 | atcattatat ttaatctctc ccgtggtcca tggtggcatt caggaagagc atcctatgca |
| 2521 | agggcacagt gggaaggaag cgctggacat ttgtcttgaa aaccactggt tctcttattg |
| 2581 | gctgaggtca tgcgtgtgcc atgcccctca gcactctaca cgtaactgct tctagtactc |
| 2641 | agcgtgtgta accgtgggac acagcgctgt agtagagcag ttgcaggatc atctggtgct |
| 2701 | gacgtatgat gtactgaaga aatactggaa ctaagacttt ttaacatgca ggttttttac |
| 2761 | tgtaatctta ataacttatt tatcaaagta gctacagaaa gcttaagtga ataatggcaa |
| 2821 | aacactgaat ctgtttgggt gttaacatta aatggtgcta caaatggtgt ttttaatagc |
| 2881 | tgaaaaatca atgccttcta tcatctagcc agtgtggtcg agggccctgg aggcactggg |
| 2941 | gtacctctga ttttacattt ctatcttaat tattcagctt agtttttaaa atgtggacat |
| 3001 | ttcaaaggcc tctggattgt agttatccac cgatgtcctt gtaggactat aatgtataga |
| 3061 | tatgcacact tacacatgtg tactgaaata ttttaagttg tgtcttagaa aagcactttg |
| 3121 | cctacctaag ctttggcaac ttgggcaatg ctaaggtact aaaacataaa aacaaaaaaa |
| 3181 | aaaaaaaa |
| 12. TET3 human DNA, Tet Methylcytosine Dioxygenase 3 | |
| (SEQ ID NO: 12) | |
| 1 | ggccccacgg tcgcctctat ccgggaactc atggaggagc ggtatggaga gaaggggaaa |
| 61 | gccatccgga tcgagaaggt catctacacg gggaaggagg gaaagagctc ccgcggttgc |
| 121 | cccattgcaa agtgggtgat ccgcaggcac acgctggagg agaagctact ctgcctggtg |
| 181 | cggcaccggg caggccacca ctgccagaac gctgtgatcg tcatcctcat cctggcctgg |
| 241 | gagggcattc cccgtagcct cggagacacc ctctaccagg agctcaccga caccctccgg |
| 301 | aagtatggga accccaccag ccggagatgc ggcctcaacg atgaccggac ctgcgcttgc |
| 361 | caaggcaaag accccaacac ctgtggtgcc tccttctcct ttggttgttc ctggagcatg |
| 421 | tacttcaacg gctgcaagta tgctcggagc aagacacctc gcaagttccg cctcgcaggg |
| 481 | gacaatccca aagaggaaga agtgctccgg aagagtttcc aggacctggc caccgaagtc |
| 541 | gctcccctgt acaagcgact ggcccctcag gcctatcaga accaggtgac caacgaggaa |
| 601 | atagcgattg actgccgtct ggggctgaag gaaggacggc ccttcgcggg ggtcacggcc |
| 661 | tgcatggact tctgtgccca cgcccacaag gaccagcata acctctacaa tgggtgcacc |
| 721 | gtggtctgca ccctgaccaa ggaagacaat cgctgcgtgg gcaagattcc cgaggatgag |
| 781 | cagctgcatg ttctccccct gtacaagatg gccaacacgg atgagtttgg tagcgaggag |
| 841 | aaccagaatg caaaggtggg cagcggagcc atccaggtgc tcaccgcctt cccccgcgag |
| 901 | gtccgacgcc tgcccgagcc tgccaagtcc tgccgccagc ggcagctgga agccagaaag |
| 961 | gcagcagccg agaagaagaa gattcagaag gagaagctga gcactccgga gaagatcaag |
| 1021 | caggaggccc tggagctggc gggcattacg tcggacccag gcctgtctct gaagggtgga |
| 1081 | ttgtcccagc aaggcctgaa gccctccctc aaggtggagc cgcagaacca cttcagctcc |
| 1141 | ttcaagtaca gcggcaacgc ggtggtggag agctactcgg tgctgggcaa ctgccggccc |
| 1201 | tccgaccctt acagcatgaa cagcgtgtac tcctaccact cctactatgc acagcccagc |
| 1261 | ctgacctccg tcaatggctt ccactccaag tacgctctcc cgtcttttag ctactatggc |
| 1321 | tttccatcca gcaaccccgt cttcccctct cagttcctgg gtcctggtgc ctgggggcat |
| 1381 | agtggcagca gtggcagttt tgagaagaag ccagacctcc acgctctgca caacagcctg |
| 1441 | agcccggcct acggtggtgc tgagtttgcc gagctgccca gccaggctgt tcccacagac |
| 1501 | gcccaccacc ccactcctca ccaccagcag cctgcgtacc caggccccaa ggagtatctg |
| 1561 | cttcccaagg cccccctact ccactcagtg tccagggacc cctccccctt tgcccagagc |
| 1621 | tccaactgct acaacagatc catcaagcaa gagccagtag acccgctgac ccaggctgag |
| 1681 | cctgtgccca gagacgctgg caagatgggc aagacacctc tgtccgaggt gtctcagaat |
| 1741 | ggaggaccca gtcacctttg gggacagtac tcaggaggcc caagcatgtc ccccaagagg |
| 1801 | actaacggtg tgggtggcag ctggggtgtg ttctcgtctg gggagagtcc tgccatcgtc |
| 1861 | cctgacaagc tcagttcctt tggggccagc tgcctggccc cttcccactt cacagatggc |
| 1921 | cagtgggggc tgttccccgg tgaggggcag caggcagctt cccactctgg aggacggctg |
| 1981 | cgaggcaaac cgtggagccc ctgcaagttt gggaacagca cctcggcctt ggctgggccc |
| 2041 | agcctgactg agaagccgtg ggcgctgggg gcaggggatt tcaactcggc cctgaaaggt |
| 2101 | agtcctgggt tccaagacaa gctgtggaac cccatgaaag gagaggaggg caggattcca |
| 2161 | gccgcagggg ccagccagct ggacagggcc tggcagtcct ttggtctgcc cctgggatcc |
| 2221 | agcgagaagc tgtttggggc tctgaagtca gaggagaagc tgtgggaccc cttcagcctg |
| 2281 | gaggaggggc cggctgagga gccccccagc aagggagcgg tgaaggagga gaagggcggt |
| 2341 | ggtggtgcgg aggaggaaga ggaggagctg tggtcggaca gtgaacacaa cttcctggac |
| 2401 | gagaacatcg gcggcgtggc cgtggcccca gcccacggct ccatcctcat cgagtgtgcc |
| 2461 | cggcgggagc tgcacgccac cacgccgctt aagaagccca accgctgcca ccccacccgc |
| 2521 | atctcgctgg tcttctacca gcacaagaac ctcaaccagc ccaaccacgg gctggccctc |
| 2581 | tgggaagcca agatgaagca gctggcggag agggcacggg cacggcagga ggaggctgcc |
| 2641 | cggctgggcc tgggccagca ggaggccaag ctctacggga agaagcgcaa gtgggggggc |
| 2701 | actgtggttg ctgagcccca gcagaaagag aagaaggggg tcgtccccac ccggcaggca |
| 2761 | ctggctgtgc ccacagactc ggcggtcacc gtgtcctcct atgcctacac gaaggtcact |
| 2821 | ggcccctaca gccgctggat ctaggtgcca gggagccagc gtacctcagc gtcgggcctg |
| 2881 | gcccgagctg tctctgtggt gcttttgccc tcatacctgg gggcgggttg ggggtgcaga |
| 2941 | agtcttttta tctctatata catatataga tgcgcatatc atatatatgt atttatggtc |
| 3001 | caaacctcag aactgacccg cccctccctt acccccactt ccccagcact ttgaagaaga |
| 3061 | aactacggct gtcgggtgat ttttccgtga tcttaatatt tatatctcca agttgtcccc |
| 3121 | cccccttgtc tggggggttt ttatttttat tttctctttg tttttaaaac tctatccttg |
| 3181 | tatatcacaa taatggaaag aaagtttata gtatcctttc acaaaggagt agttttaaaa |
| 3241 | aaaaaaaaaa a |
| 13. Sirt1 human DNA,, Sirtuin 1 | |
| (SEQ ID NO: 13) | |
| 1 | aagacgacga cgacgagggc gaggaggagg aagaggcggc ggcggcggcg attgggtacc |
| 61 | gagataacct tctgttcggt gatgaaatta tcactaatgg ttttcattcc tgtgaaagtg |
| 121 | atgaggagga tagagcctca catgcaagct ctagtgactg gactccaagg ccacggatag |
| 181 | gtccatatac ttttgtccag caacatctta tgattggcac agatcctcga acaattctta |
| 241 | aagatttatt gccggaaaca atacctccac ctgagttgga tgatatgaca ctgtggcaga |
| 301 | ttgttattaa tatcctttca gaaccaccaa aaaggaaaaa aagaaaagat attaatacaa |
| 361 | ttgaagatgc tgtgaaatta ctgcaagagt gcaaaaaaat tatagttcta actggagctg |
| 421 | gggtgtctgt ttcatgtgga atacctgact tcaggtcaag ggatggtatt tatgctcgcc |
| 481 | ttgctgtaga cttcccagat cttccagatc ctcaagcgat gtttgatatt gaatatttca |
| 541 | gaaaagatcc aagaccattc ttcaagtttg caaaggaaat atatcctgga caattccagc |
| 601 | catctctctg tcacaaattc atagccttgt cagataagga aggaaaacta cttcgcaact |
| 661 | atacccagaa catagacacg ctggaacagg ttgcgggaat ccaaaggata attcagtgtc |
| 721 | atggttcctt tgcaacagca tcttgcctga tttgtaaata caaagttgac tgtgaagctg |
| 781 | tacgaggagc tctttttagt caggtagttc ctcgatgtcc taggtgccca gctgatgaac |
| 841 | cgcttgctat catgaaacca gagattgtgt tttttggtga aaatttacca gaacagtttc |
| 901 | atagagccat gaagtatgac aaagatgaag ttgacctcct cattgttatt gggtcttccc |
| 961 | tcaaagtaag accagtagca ctaattccaa gttccatacc ccatgaagtg cctcagatat |
| 1021 | taattaatag agaacctttg cctcatctgc attttgatgt agagcttctt ggagactgtg |
| 1081 | atgtcataat taatgaattg tgtcataggt taggtggtga atatgccaaa ctttgctgta |
| 1141 | accctgtaaa gctttcagaa attactgaaa aacctccacg aacacaaaaa gaattggctt |
| 1201 | atttgtcaga gttgccaccc acacctcttc atgtttcaga agactcaagt tcaccagaaa |
| 1261 | gaacttcacc accagattct tcagtgattg tcacactttt agaccaagca gctaagagta |
| 1321 | atgatgattt agatgtgtct gaatcaaaag gttgtatgga agaaaaacca caggaagtac |
| 1381 | aaacttctag gaatgttgaa agtattgctg aacagatgga aaatccggat ttgaagaatg |
| 1441 | ttggttctag tactggggag aaaaatgaaa gaacttcagt ggctggaaca gtgagaaaat |
| 1501 | gctggcctaa tagagtggca aaggagcaga ttagtaggcg gcttgatggt aatcagtatc |
| 1561 | tgtttttgcc accaaatcgt tacattttcc atggcgctga ggtatattca gactctgaag |
| 1621 | atgacgtctt atcctctagt tcttgtggca gtaacagtga tagtgggaca tgccagagtc |
| 1681 | caagtttaga agaacccatg gaggatgaaa gtgaaattga agaattctac aatggcttag |
| 1741 | aagatgagcc tgatgttcca gagagagctg gaggagctgg atttgggact gatggagatg |
| 1801 | atcaagaggc aattaatgaa gctatatctg tgaaacagga agtaacagac atgaactatc |
| 1861 | catcaaacaa atcatagtgt aataattgtg caggtacagg aattgttcca ccagcattag |
| 1921 | gaactttagc atgtcaaaat gaatgtttac ttgtgaactc gatagagcaa ggaaaccaga |
| 1981 | aaggtgtaat atttataggt tggtaaaata gattgttttt catggataat ttttaacttc |
| 2041 | attatttctg tacttgtaca aactcaacac taactttttt ttttttaaaa aaaaaaaggt |
| 2101 | actaagtatc ttcaatcagc tgttggtcaa gactaacttt cttttaaagg ttcatttgta |
| 2161 | tgataaattc atatgtgtat atataatttt tttttgtttt gtctagtgag tttcaacatt |
| 2221 | tttaaagttt tcaaaaagcc atcggaatgt taaattaatg taaagggaac agctaatcta |
| 2281 | gaccaaagaa tggtattttc acttttcttt gtaacattga atggtttgaa gtactcaaaa |
| 2341 | tctgttacgc taaacttttg attctttaac acaattattt ttaaacactg gcattttcca |
| 2401 | aaactgtggc agctaacttt ttaaaatctc aaatgacatg cagtgtgagt agaaggaagt |
| 2461 | caacaatatg tggggagagc actcggttgt ctttactttt aaaagtaata cttggtgcta |
| 2521 | agaatttcag gattattgta tttacgttca aatgaagatg gcttttgtac ttcctgtgga |
| 2581 | catgtagtaa tgtctatatt ggctcataaa actaacctga aaaacaaata aatgctttgg |
| 2641 | aaatgtttca gttgctttag aaacattagt gcctgcctgg atccccttag ttttgaaata |
| 2701 | tttgccattg ttgtttaaat acctatcact gtggtagagc ttgcattgat cttttccaca |
| 2761 | agtattaaac tgccaaaatg tgaatatgca aagcctttct gaatctataa taatggtact |
| 2821 | tctactgggg agagtgtaat attttggact gctgttttcc attaatgagg agagcaacag |
| 2881 | gcccctgatt atacagttcc aaagtaataa gatgttaatt gtaattcagc cagaaagtac |
| 2941 | atgtctccca ttgggaggat ttggtgttaa ataccaaact gctagcccta gtattatgga |
| 3001 | gatgaacatg atgatgtaac ttgtaatagc agaatagtta atgaatgaaa ctagttctta |
| 3061 | taatttatct ttatttaaaa gcttagcctg ccttaaaact agagatcaac tttctcagct |
| 3121 | gcaaaagctt ctagtctttc aagaagttca tactttatga aattgcacag taagcattta |
| 3181 | tttttcagac catttttgaa catcactcct aaattaataa agtattcctc tgttgcttta |
| 3241 | gtatttatta caataaaaag ggtttgaaat atagctgttc tttatgcata aaacacccag |
| 3301 | ctaggaccat tactgccaga gaaaaaaatc gtattgaatg gccatttccc tacttataag |
| 3361 | atgtctcaat ctgaatttat ttggctacac taaagaatgc agtatattta gttttccatt |
| 3421 | tgcatgatgt ttgtgtgcta tagatgatat tttaaattga aaagtttgtt ttaaattatt |
| 3481 | tttacagtga agactgtttt cagctctttt tatattgtac atagtctttt atgtaattta |
| 3541 | ctggcatatg ttttgtagac tgtttaatga ctggatatct tccttcaact tttgaaatac |
| 3601 | aaaaccagtg ttttttactt gtacactgtt ttaaagtcta ttaaaattgt catttgactt |
| 3661 | ttttctgtta acttaaaaaa aaaaaaaaaa a |
| 14. Sirt6 human DNA, Sirtuin 6 | |
| (SEQ ID NO: 14) | |
| 1 | ggcagtcgag gatgtcggtg aattacgcgg cggggctgtc gccgtacgcg gacaagggca |
| 61 | agtgcggcct cccggagatc ttcgaccccc cggaggagct ggagcggaag gtgtgggaac |
| 121 | tggcgaggct ggtctggcag tcttccaatg tggtgttcca cacgggtgcc ggcatcagca |
| 181 | ctgcctctgg catccccgac ttcaggggtc cccacggagt ctggaccatg gaggagcgag |
| 241 | gtctggcccc caagttcgac accacctttg agagcgcgcg gcccacgcag acccacatgg |
| 301 | cgctggtgca gctggagcgc gtgggcctcc tccgcttcct ggtcagccag aacgtggacg |
| 361 | ggctccatgt gcgctcaggc ttccccaggg acaaactggc agagctccac gggaacatgt |
| 421 | ttgtggaaga atgtgccaag tgtaagacgc agtacgtccg agacacagtc gtgggcacca |
| 481 | tgggcctgaa ggccacgggc cggctctgca ccgtggctaa ggcaaggggg ctgcgagcct |
| 541 | gcaggaacgc cgacctgtcc atcacgctgg gtacatcgct gcagatccgg cccagcggga |
| 601 | acctgccgct ggctaccaag cgccggggag gccgcctggt catcgtcaac ctgcagccca |
| 661 | ccaagcacga ccgccatgct gacctccgca tccatggcta cgttgacgag gtcatgaccc |
| 721 | ggctcatgaa gcacctgggg ctggagatcc ccgcctggga cggcccccgt gtgctggaga |
| 781 | gggcgctgcc acccctgccc cgcccgccca cccccaagct ggagcccaag gaggaatctc |
| 841 | ccacccggat caacggctct atccccgccg gccccaagca ggagccctgc gcccagcaca |
| 901 | acggctcaga gcccgccagc cccaaacggg agcggcccac cagccctgcc ccccacagac |
| 961 | cccccaaaag ggtgaaggcc aaggcggtcc ccagctgacc agggtgcttg gggagggtgg |
| 1021 | ggctttttgt agaaactgtg gattcttttt ctctcgtggt ctcactttgt tacttgtttc |
| 1081 | tgtccccggg agcctcaggg ctctgagagc tgtgctccag gccaggggtt acacctgccc |
| 1141 | tccgtggtcc ctccctgggc tccaggggcc tctggtgcgg ttccgggaag aagccacacc |
| 1201 | ccagaggtga cagctgagcc cctgccacac cccagcctct gacttgctgt gttgtccaga |
| 1261 | ggtgaggctg ggccctccct ggtctccagc ttaaacagga gtgaactccc tctgtcccca |
| 1321 | gggcctccct tctgggcccc ctacagccca ccctacccct cctccatggg ccctgcagga |
| 1381 | ggggagaccc accttgaagt gggggatcag tagaggcttg cactgccttt ggggctggag |
| 1441 | ggagacgtgg gtccaccagg cttctggaaa agtcctcaat gcaataaaaa caatttcttt |
| 1501 | cttgcaaaaa aaaaaaaaaa aaaaaaaaaa aaaaaaaaaa aaaaaaaaaa aaaaaaaaaa |
| 1561 | aaaaaaaaaa aaaaaaaaaa aaaaa |
| 15. Pck1 human DNA, Phosphoenolpyruvate Carboxykinase 1 | |
| (SEQ ID NO: 15) | |
| 1 | ggggacggcc ttcccactgg gaacacaaac ttgctggcgg gaagagcccg gaaagaaacc |
| 61 | tgtggatctc ccttcgagat catccaaaga gaagaaaggt gacctcacat tcgtgcccct |
| 121 | tagcagcact ctgcagaaat gcctcctcag ctgcaaaacg gcctgaacct ctcggccaaa |
| 181 | gttgtccagg gaagcctgga cagcctgccc caggcagtga gggagtttct cgagaataac |
| 241 | gctgagctgt gtcagcctga tcacatccac atctgtgacg gctctgagga ggagaatggg |
| 301 | cggcttctgg gccagatgga ggaagagggc atcctcaggc ggctgaagaa gtatgacaac |
| 361 | tgctggttgg ctctcactga ccccagggat gtggccagga tcgaaagcaa gacggttatc |
| 421 | gtcacccaag agcaaagaga cacagtgccc atccccaaaa caggcctcag ccagctcggt |
| 481 | cgctggatgt cagaggagga ttttgagaaa gcgttcaatg ccaggttccc agggtgcatg |
| 541 | aaaggtcgca ccatgtacgt catcccattc agcatggggc cgctgggctc acctctgtcg |
| 601 | aagatcggca tcgagctgac ggattcgccc tacgtggtgg ccagcatgcg gatcatgacg |
| 661 | cggatgggca cgcccgtcct ggaagcactg ggcgatgggg agtttgtcaa atgcctccat |
| 721 | tctgtggggt gccctctgcc tttacaaaag cctttggtca acaactggcc ctgcaacccg |
| 781 | gagctgacgc tcatcgccca cctgcctgac cgcagagaga tcatctcctt tggcagtggg |
| 841 | tacggcggga actcgctgct cgggaagaag tgctttgctc tcaggatggc cagccggctg |
| 901 | gccaaggagg aagggtggct ggcagagcac atgctggttc tgggtataac caaccctgag |
| 961 | ggtgagaaga agtacctggc ggccgcattt cccagcgcct gcgggaagac caacctggcc |
| 1021 | atgatgaacc ccagcctccc cgggtggaag gttgagtgcg tcggggatga cattgcctgg |
| 1081 | atgaagtttg acgcacaagg tcatttaagg gccatcaacc cagaaaatgg ctttttcggt |
| 1141 | gtcgctcctg ggacttcagt gaagaccaac cccaatgcca tcaagaccat ccagaagaac |
| 1201 | acaatcttta ccaatgtggc cgagaccagc gacgggggcg tttactggga aggcattgat |
| 1261 | gagccgctag cttcaggtgt caccatcacg tcctggaaga ataaggagtg gagctcagag |
| 1321 | gatggggaac cttgtgccca ccccaactcg aggttctgca cccctgccag ccagtgcccc |
| 1381 | atcattgatg ctgcctggga gtctccggaa ggtgttccca ttgaaggcat tatctttgga |
| 1441 | ggccgtagac ctgctggtgt ccctctagtc tatgaagctc tcagctggca acatggagtc |
| 1501 | tttgtggggg cggccatgag atcagaggcc acagcggctg cagaacataa aggcaaaatc |
| 1561 | atcatgcatg acccctttgc catgcggccc ttctttggct acaacttcgg caaatacctg |
| 1621 | gcccactggc ttagcatggc ccagcaccca gcagccaaac tgcccaagat cttccatgtc |
| 1681 | aactggttcc ggaaggacaa ggaaggcaaa ttcctctggc caggctttgg agagaactcc |
| 1741 | agggtgctgg agtggatgtt caaccggatc gatggaaaag ccagcaccaa gctcacgccc |
| 1801 | ataggctaca tccccaagga ggatgccctg aacctgaaag gcctggggca catcaacatg |
| 1861 | atggagcttt tcagcatctc caaggaattc tgggagaagg aggtggaaga catcgagaag |
| 1921 | tatctggagg atcaagtcaa tgccgacctc ccctgtgaaa tcgagagaga gatccttgcc |
| 1981 | ttgaagcaaa gaataagcca gatgtaatca gggcctgagt gctttacctt taaaatcatt |
| 2041 | ccctttccca tccataaggt gcagtaggag caagagaggg caagtgttcc caaattgacg |
| 2101 | ccaccataat aatcatcacc acaccgggag cagatctgaa aggcacactt tgattttttt |
| 2161 | aaggataaga accacagaac actgggtagt agctaatgaa attgagaagg gaaatcttag |
| 2221 | catgcctcca aaaattcaca tccaatgcat agtttgttca aatttaaggt tactcaggca |
| 2281 | ttgatctttt cagtgttttt tcactttagc tatgtggatt agctagaatg cacaccaaaa |
| 2341 | aaatacttga gctgtatata tatgtgtgtg tgtgtgtgtg tgtgtgtgtg catgtatgtg |
| 2401 | cacatgtgtc tgtgtggtat atttgtgtat gtgtatttgt atgtactgtt attgaaaata |
| 2461 | tatttaatac ctttggaaaa atcttgggca agatgaccta ctagttttcc ttgaaaaaaa |
| 2521 | gttgctttgt tattaatatt gtgcttaaat tatttttata caccattgtt ccttaccttt |
| 2581 | acataattgc aatatttccc ccttactact tcttggaaaa aaattacaaa atgaagtttt |
| 2641 | aaaaaaaaaa aaaaaaaaaa aaaaaaaaa |
| 16. Pparg human DNA, Peroxisome Proliferator Activated Receptor Gamma | |
| (SEQ ID NO: 16) | |
| 1 | ccagaagcct gcatttctgc attctgctta attccctttc cttagatttg aaagaagcca |
| 61 | acactaaacc acaaatatac aacaaggcca ttttctcaaa cgagagtcag cctttaacga |
| 121 | aatgaccatg gttgacacag agatgccatt ctggcccacc aactttggga tcagctccgt |
| 181 | ggatctctcc gtaatggaag accactccca ctcctttgat atcaagccct tcactactgt |
| 241 | tgacttctcc agcatttcta ctccacatta cgaagacatt ccattcacaa gaacagatcc |
| 301 | agtggttgca gattacaagt atgacctgaa acttcaagag taccaaagtg caatcaaagt |
| 361 | ggagcctgca tctccacctt attattctga gaagactcag ctctacaata agcctcatga |
| 421 | agagccttcc aactccctca tggcaattga atgtcgtgtc tgtggagata aagcttctgg |
| 481 | atttcactat ggagttcatg cttgtgaagg atgcaagggt ttcttccgga gaacaatcag |
| 541 | attgaagctt atctatgaca gatgtgatct taactgtcgg atccacaaaa aaagtagaaa |
| 601 | taaatgtcag tactgtcggt ttcagaaatg ccttgcagtg gggatgtctc ataatgccat |
| 661 | caggtttggg cggatgccac aggccgagaa ggagaagctg ttggcggaga tctccagtga |
| 721 | tatcgaccag ctgaatccag agtccgctga cctccgggcc ctggcaaaac atttgtatga |
| 781 | ctcatacata aagtccttcc cgctgaccaa agcaaaggcg agggcgatct tgacaggaaa |
| 841 | gacaacagac aaatcaccat tcgttatcta tgacatgaat tccttaatga tgggagaaga |
| 901 | taaaatcaag ttcaaacaca tcacccccct gcaggagcag agcaaagagg tggccatccg |
| 961 | catctttcag ggctgccagt ttcgctccgt ggaggctgtg caggagatca cagagtatgc |
| 1021 | caaaagcatt cctggttttg taaatcttga cttgaacgac caagtaactc tcctcaaata |
| 1081 | tggagtccac gagatcattt acacaatgct ggcctccttg atgaataaag atggggttct |
| 1141 | catatccgag ggccaaggct tcatgacaag ggagtttcta aagagcctgc gaaagccttt |
| 1201 | tggtgacttt atggagccca agtttgagtt tgctgtgaag ttcaatgcac tggaattaga |
| 1261 | tgacagcgac ttggcaatat ttattgctgt cattattctc agtggagacc gcccaggttt |
| 1321 | gctgaatgtg aagcccattg aagacattca agacaacctg ctacaagccc tggagctcca |
| 1381 | gctgaagctg aaccaccctg agtcctcaca gctgtttgcc aagctgctcc agaaaatgac |
| 1441 | agacctcaga cagattgtca cggaacacgt gcagctactg caggtgatca agaagacgga |
| 1501 | gacagacatg agtcttcacc cgctcctgca ggagatctac aaggacttgt actagcagag |
| 1561 | agtcctgagc cactgccaac atttcccttc ttccagttgc actattctga gggaaaatct |
| 1621 | gacacctaag aaatttactg tgaaaaagca ttttaaaaag aaaaggtttt agaatatgat |
| 1681 | ctattttatg catattgttt ataaagacac atttacaatt tacttttaat attaaaaatt |
| 1741 | accatattat gaaaaaaaaa aaaaaaa |
| 17. Cisd2 human DNA, CDGSH Iron Sulfur Domain 2 | |
| (SEQ ID NO: 17) | |
| 1 | ccacgcgtcc gggctcggga gaggagtgga cgccgctggc caggatggtg ctggagagcg |
| 61 | tggcccgtat cgtgaaggtg cagctccctg catatctgaa gcggctccca gtccctgaaa |
| 121 | gcattaccgg gttcgctagg ctcacagttt cagaatggct tcggttattg cctttccttg |
| 181 | gtgtactcgc acttcttggc taccttgcag ttcgtccatt cctcccgaag aagaaacaac |
| 241 | agaaggatag cttgattaat cttaaaatac aaaaggaaaa tccgaaagta gtgaatgaaa |
| 301 | taaacattga agatttgtgt cttactaaag cagcttattg taggtgttgg cgttctaaaa |
| 361 | cgtttcctgc ctgcgatggt tcacataata aacacaatga attgacagga gataatgtgg |
| 421 | gtccactaat actgaagaag aaagaagtat aataataata acaatatttt ctcattcttt |
| 481 | gtgtatagaa aattttaaaa tggtggtctt aattattact actggttgaa caattatttc |
| 541 | ttccaattta ttttcttcct gcactactgt ttgtatttga tcctttgtct attcagtcac |
| 601 | ttaattagaa attaaattgt caagcctctt attctgactt caaagaatta atgtatcttc |
| 661 | caacaataaa atcacttctg attttaatct aggaaaacct aaattgtggc tatggatcca |
| 721 | aagctgtttg tttctttgaa tatcaatatt ttcaacagga tcttgtattt aaaattccca |
| 781 | cctacattgt taaatatgtt attttttcat atctcttttg gttttgataa tctgaagtgt |
| 841 | ttttttctcg ttttggcctt ccaaactgca tttggttagg tgaattaaga aaaatattgc |
| 901 | catcaagaat tacttgtgtt ttcacagaga tagactcttt gctttataga gattgttgtg |
| 961 | tatttaatat gaatatccca gctttagaaa agaagtaaac tggatacaaa aagttccatt |
| 1021 | gaggaacagt tatttacagt ataaaagatt tgtttacttt acaaaaggct tgtgtctgtg |
| 1081 | tgtgtgtgtg tgtgtgtgtg tgtgtgtatt ttaaactgac tcagtgacag ctggggtgga |
| 1141 | atggcaagaa cacttacaac caaactcatg ggctgctgca atttgaagat caattggtaa |
| 1201 | taaacataag acattaattc atattaaaat agttcagtgt tcaaaattgt gtttatgtgg |
| 1261 | atatttttct ctttttaaca ctataaacca ttaaaataca gtcatccctt gtatacgcta |
| 1321 | gggactggtt ccagggccac acatatacca aaatctgccc atactcaagt cccacagaaa |
| 1381 | gtcttgcaga acccatatgt agaaaagttg gccctccagt tgaccctccg tacacatgag |
| 1441 | tttcacatcc catgcacaaa tgctgatctg tgtgacctca cctgcatttg attgaaaaaa |
| 1501 | gtatgcgcgt aagtgtaccc acccagttca aacccgtgtg taagggtcaa ctgtacaaaa |
| 1561 | aagtttgtga aataaacgta ctggagaatc tttaaaaaaa aaaaaaaaaa aaaaaaa |
| 18. MDH1 human DNA, Malate Dehydrogenase 1 | |
| (SEQ ID NO: 18) | |
| 1 | ctgactctct gaggctcatt ttgcagttgt tgaaattgtc cccgcagttt tcaatcatgt |
| 61 | ctgaaccaat cagagtcctt gtgactggag cagctggtca aattgcatat tcactgctgt |
| 121 | acagtattgg aaatggatct gtctttggta aagatcagcc tataattctt gtgctgttgg |
| 181 | atatcacccc catgatgggt gtcctggacg gtgtcctaat ggaactgcaa gactgtgccc |
| 241 | ttcccctcct gaaagatgtc atcgcaacag ataaagaaga cgttgccttc aaagacctgg |
| 301 | atgtggccat tcttgtgggc tccatgccaa gaagggaagg catggagaga aaagatttac |
| 361 | tgaaagcaaa tgtgaaaatc ttcaaatccc agggtgcagc cttagataaa tacgccaaga |
| 421 | agtcagttaa ggttattgtt gtgggtaatc cagccaatac caactgcctg actgcttcca |
| 481 | agtcagctcc atccatcccc aaggagaact tcagttgctt gactcgtttg gatcacaacc |
| 541 | gagctaaagc tcaaattgct cttaaacttg gtgtgactgc taatgatgta aagaatgtca |
| 601 | ttatctgggg aaaccattcc tcgactcagt atccagatgt caaccatgcc aaggtgaaat |
| 661 | tgcaaggaaa ggaagttggt gtttatgaag ctctgaaaga tgacagctgg ctcaagggag |
| 721 | aatttgtcac gactgtgcag cagcgtggcg ctgctgtcat caaggctcga aaactatcca |
| 781 | gtgccatgtc tgctgcaaaa gccatctgtg accacgtcag ggacatctgg tttggaaccc |
| 841 | cagagggaga gtttgtgtcc atgggtgtta tctctgatgg caactcctat ggtgttcctg |
| 901 | atgatctgct ctactcattc cctgttgtaa tcaagaataa gacctggaag tttgttgaag |
| 961 | gtctccctat taatgatttc tcacgtgaga agatggatct tactgcaaag gaactgacag |
| 1021 | aagaaaaaga aagtgctttt gaatttcttt cctctgcctg actagacaat gatgttacta |
| 1081 | aatgcttcaa agctgaagaa tctaaatgtc gtctttgact caagtaccaa ataataataa |
| 1141 | tgctatactt aaattacttg tgaaaaacaa cacattttaa agattacgtg cttcttggta |
| 1201 | caggtttgtg aatgacagtt tatcgtcatg ctgttagtgt gcattctaaa taaatatata |
| 1261 | ttcaaatgaa aaaaaaaaaa aaaaaa |
| 19. MDH2 human DNA, Malate Dehydrogenase 2 | |
| (SEQ ID NO: 19) | |
| 1 | gccagtcggt gcccctcccg ctccagccat gctctccgcc ctcgcccggc ctgtcagcgc |
| 61 | tgctctccgc cgcagcttca gcacctcggc ccagaacaat gctaaagtag ctgtgctagg |
| 121 | ggcctctgga ggcatcgggc agccactttc acttctcctg aagaacagcc ccttggtgag |
| 181 | ccgcctgacc ctctatgata tcgcgcacac acccggagtg gccgcagatc tgagccacat |
| 241 | cgagaccaaa gccgctgtga aaggctacct cggacctgaa cagctgcctg actgcctgaa |
| 301 | aggttgtgat gtggtagtta ttccggctgg agtccccaga aagccaggca tgacccggga |
| 361 | cgacctgttc aacaccaatg ccacgattgt ggccaccctg accgctgcct gtgcccagca |
| 421 | ctgcccggaa gccatgatct gcgtcattgc caatccggtt aattccacca tccccatcac |
| 481 | agcagaagtt ttcaagaagc atggagtgta caaccccaac aaaatcttcg gcgtgacgac |
| 541 | cctggacatc gtcagagcca acacctttgt tgcagagctg aagggtttgg atccagctcg |
| 601 | agtcaacgtc cctgtcattg gtggccatgc tgggaagacc atcatccccc tgatctctca |
| 661 | gtgcaccccc aaggtggact ttccccagga ccagctgaca gcactcactg ggcggatcca |
| 721 | ggaggccggc acggaggtgg tcaaggctaa agccggagca ggctctgcca ccctctccat |
| 781 | ggcgtatgcc ggcgcccgct ttgtcttctc ccttgtggat gcaatgaatg gaaaggaagg |
| 841 | tgttgtggaa tgttccttcg ttaagtcaca ggaaacggaa tgtacctact tctccacacc |
| 901 | gctgctgctt gggaaaaagg gcatcgagaa gaacctgggc atcggcaaag tctcctcttt |
| 961 | tgaggagaag atgatctcgg atgccatccc cgagctgaag gcctccatca agaaggggga |
| 1021 | agatttcgtg aagaccctga agtgagccgc tgtgacgggt ggccagtttc cttaatttat |
| 1081 | gaaggcatca tgtcactgca aagccgttgc agataaactt tgtattttaa tttgctttgg |
| 1141 | tgatgattac tgtattgaca tcatcatgcc ttccaaattg tgggtggctc tgtgggcgca |
| 1201 | tcaataaaag ccgtccttga ttttaaaaaa aaaaaaaaaa aaaa |
| 20. Aco1 human DNA, Aconitase 1 | |
| (SEQ ID NO: 20) | |
| 1 | gccgtgcagt cggaggaaca cgtggccatc agtaatcatg agcaacccat tcgcacacct |
| 61 | tgctgagcca ttggatcctg tacaaccagg aaagaaattc ttcaatttga ataaattgga |
| 121 | ggattcaaga tatgggcgct taccattttc gatcagagtt cttctggaag cagccattcg |
| 181 | gaattgtgat gagtttttgg tgaagaaaca ggatattgaa aatattctac attggaatgt |
| 241 | cacgcagcac aagaacatag aagtgccatt taagcctgct cgtgtcatcc tgcaggactt |
| 301 | tacgggtgtg cccgctgtgg ttgactttgc tgcaatgcgt gatgctgtga aaaagttagg |
| 361 | aggagatcca gagaaaataa accctgtctg ccctgctgat cttgtaatag atcattccat |
| 421 | ccaggttgat ttcaacagaa gggcagacag tttacagaag aatcaagacc tggaatttga |
| 481 | aagaaataga gagcgatttg aatttttaaa gtggggttcc caggcttttc acaacatgcg |
| 541 | gattattccc cctggctcag gaatcatcca ccaggtgaat ttggaatatt tggcaagagt |
| 601 | ggtatttgat caggatggat attattaccc agacagcctc gtgggcacag actcgcacac |
| 661 | taccatgatt gatggcttgg gcattcttgg ttggggtgtc ggtggtattg aagcagaagc |
| 721 | tgtcatgctg ggtcagccaa tcagtatggt gcttcctcag gtgattggct acaggctgat |
| 781 | ggggaagccc caccctctgg taacatccac tgacatcgtg ctcaccatta ccaagcacct |
| 841 | ccgccaggtt ggggtagtgg gcaaatttgt cgagttcttc gggcctggag tagcccagtt |
| 901 | gtccattgct gaccgagcta cgattgctaa catgtgtcca gagtacggag caactgctgc |
| 961 | ctttttccca gttgatgaag ttagtatcac gtacctggtg caaacaggtc gtgatgaaga |
| 1021 | aaaattaaag tatattaaaa aatatcttca ggctgtagga atgtttcgag atttcaatga |
| 1081 | cccttctcaa gacccagact tcacccaggt tgtggaatta gatttgaaaa cagtagtgcc |
| 1141 | ttgctgtagt ggacccaaaa ggcctcagga caaagttgct gtgtccgaca tgaaaaagga |
| 1201 | ctttgagagc tgccttggag ccaagcaagg atttaaagga ttccaagttg ctcctgaaca |
| 1261 | tcataatgac cataagacct ttatctatga taacactgaa ttcacccttg ctcatggttc |
| 1321 | tgtggtcatt gctgccatta ctagctgcac aaacaccagt aatccgtctg tgatgttagg |
| 1381 | ggcaggattg ttagcaaaga aagctgtgga tgctggcctg aacgtgatgc cttacatcaa |
| 1441 | aactagcctg tctcctggga gtggcgtggt cacctactac ctacaagaaa gcggagtcat |
| 1501 | gccttatctg tctcagcttg ggtttgacgt ggtgggctat ggctgcatga cctgcattgg |
| 1561 | caacagtggg cctttacctg aacctgtggt agaagccatc acacagggag accttgtagc |
| 1621 | tgttggagta ctatctggaa acaggaattt tgaaggtcga gttcacccca acacccgggc |
| 1681 | caactattta gcctctcccc ccttagtaat agcatatgca attgctggaa ccatcagaat |
| 1741 | cgactttgag aaagagccat tgggagtaaa tgcaaaggga cagcaggtat ttctgaaaga |
| 1801 | tatctggccg actagagacg agatccaggc agtggagcgt cagtatgtca tcccggggat |
| 1861 | gtttaaggaa gtctatcaga aaatagagac tgtgaatgaa agctggaatg ccttagcaac |
| 1921 | cccatcagat aagctgtttt tctggaattc caaatctacg tatatcaaat caccaccatt |
| 1981 | ctttgaaaac ctgactttgg atcttcagcc ccctaaatct atagtggatg cctatgtgct |
| 2041 | gctaaatttg ggagattcgg taacaactga ccacatctcc ccagctggaa atattgcaag |
| 2101 | aaacagtcct gctgctcgct acttaactaa cagaggccta actccacgag aattcaactc |
| 2161 | ctatggctcc cgccgaggta atgacgccgt catggcacgg ggaacatttg ccaacattcg |
| 2221 | cttgttaaac agatttttga acaagcaggc accacagact atccatctgc cttctgggga |
| 2281 | aatccttgat gtgtttgatg ctgctgagcg gtaccagcag gcaggccttc ccctgatcgt |
| 2341 | tctggctggc aaagagtacg gtgcaggcag ctcccgagac tgggcagcta agggcccttt |
| 2401 | cctgctggga atcaaagccg tcctggccga gagctacgag cgcattcacc gcagtaacct |
| 2461 | ggttgggatg ggtgtgatcc cacttgaata tctccctggt gagaatgcag atgccctggg |
| 2521 | gctcacaggg caagaacgat acactatcat tattccagaa aacctcaaac cacaaatgaa |
| 2581 | agtccaggtc aagctggata ctggcaagac cttccaggct gtcatgaggt ttgacactga |
| 2641 | tgtggagctc acttatttcc tcaacggggg catcctcaac tacatgatcc gcaagatggc |
| 2701 | caagtaggag acgtgcactt ggtgctgcgc ccagggagga agccgcacca ccagccagcg |
| 2761 | caggccctgg tggagaggcc tccctggctg cctctgggag gggtgctgcc ttgtagatgg |
| 2821 | agcaagtgag cactgagggt ctggtgccaa tcctgtaggc acaaaaccag aagtttctac |
| 2881 | attctctatt tttgttaatc atcttctctt tttccagaat ttggaagcta gaatggtggg |
| 2941 | aatgtcagta gtgccagaaa gagagaacca agcttgtctt taaagttact gatcacagga |
| 3001 | cgttgctttt tcactgtttc ctattaatct tcagctgaac acaagcaaac cttctcagga |
| 3061 | ggtgtctcct accctcttat tgttcctctt acgctctgct caatgaaacc ttcctcttga |
| 3121 | gggtcatttt cctttctgta ttaattatac cagtgttaag tgacatagat aagaactttg |
| 3181 | cacacttcaa atcagagcag tgattctctc ttctctcccc ttttccttca gagtgaatca |
| 3241 | tccagactcc tcatggatag gtcgggtgtt aaagttgttt tgattatgta ccttttgata |
| 3301 | gatccacata aaaagaaatg tgaagttttc ttttactatc ttttcattta tcaagcagag |
| 3361 | acctttgttg ggaggcggtt tgggagaaca catttctaat ttgaatgaaa tgaaatctat |
| 3421 | tttcagtgaa aaaaaaaaaa aaa |
| 21. Aco2 human DNA, Aconitase 2 | |
| (SEQ ID NO: 21) | |
| 1 | gtcctcatct ttgtcagtgc acaaaatggc gccctacagc ctactggtga ctcggctgca |
| 61 | gaaagctctg ggtgtgcggc agtaccatgt ggcctcagtc ctgtgccaac gggccaaggt |
| 121 | ggcgatgagc cactttgagc ccaacgagta catccattat gacctgctag agaagaacat |
| 181 | taacattgtt cgcaaacgac tgaaccggcc gctgacactc tcggagaaga ttgtgtatgg |
| 241 | acacctggat gaccccgcca gccaggaaat tgagcgaggc aagtcgtacc tgcggctgcg |
| 301 | gccggaccgt gtggccatgc aggatgcgac ggcccagatg gccatgctcc agttcatcag |
| 361 | cagcgggctg tccaaggtgg ctgtgccatc caccatccac tgtgaccatc tgattgaagc |
| 421 | ccaggttggg ggcgagaaag acctgcgccg ggccaaggac atcaaccagg aagtttataa |
| 481 | tttcctggca actgcaggtg ccaaatatgg cgtgggcttc tggaagcctg gatctggaat |
| 541 | cattcaccag attattctgg aaaactatgc gtaccctggt gttcttctga ttggcactga |
| 601 | ctcccacacc cccaatggtg gcggccttgg gggcatctgc attcgagttg ggggtgccga |
| 661 | tgctgtggat gtcatggctg ggatcccctg ggagttgaag tgccccaagg tgattggcgt |
| 721 | gaagctgacg ggctctctct ccggttggtc ctcacccaaa gatgtgatcc tgaaggtggc |
| 781 | aggcatcctc acggtgaaag gtggcacagg tgcaatcgtg gaataccacg ggcatggtgt |
| 841 | agactccatc tcctgcactg gcatggcgac aatctgcaac atgggtgcag aaattggggc |
| 901 | caccacttcc gtgttccctt acaaccacag gatgaagaag tacctgagca agaccggccg |
| 961 | ggaagacatt gccaatctag ctgatgaatt caaggatcac ttggtgcctg accctggctg |
| 1021 | ccattatgac caactaattg aaattaacct cagtgagctg aagccacaca tcaatgggcc |
| 1081 | cttcacccct gacctggctc accctgtggc agaagtgggc aaggtggcag agaaggaagg |
| 1141 | atggcctctg gacatccgag tgggtctaat tggtagctgc accaattcaa gctatgaaga |
| 1201 | tatggggcgc tcagcagctg tggccaagca ggcactggcc catggcctca agtgcaagtc |
| 1261 | ccagttcacc atcactccag gttccgagca gatccgcgcc accattgagc gggacggcta |
| 1321 | tgcacagatc ttgagggatc tgggtggcat tgtcctggcc aatgcttgtg gcccctgcat |
| 1381 | tggccagtgg gacaggaagg acatcaagaa gggggagaag aacacaatcg tcacctcgta |
| 1441 | caacaggaac ttcacgggcc gcaacgacgc aaaccccgag acccatgcct ttgtcacgtc |
| 1501 | cccagagatt gtcacagccc tggccattgc gggaaccctc aagttcaacc cagagaccga |
| 1561 | ctacctgacg ggcacggatg gcaagaagtt caggctggag gctccggatg cagatgagct |
| 1621 | tcccaaaggg gagtttgacc cagggcagga cacctaccag cacccaccca aggacagcag |
| 1681 | cgggcagcat gtggacgtga gccccaccag ccagcgcctg cagctcctgg agccttttga |
| 1741 | caagtgggat ggcaaggacc tggaggacct gcagatcctc atcaaggtca aagggaagtg |
| 1801 | taccactgac cacatctcag ctgctggccc ctggctcaag ttccgtgggc acttggataa |
| 1861 | catctccaac aacctgctca ttggtgccat caacattgaa aacggcaagg ccaactccgt |
| 1921 | gcgcaatgcc gtcactcagg agtttggccc cgtccctgac actgcccgct actacaagaa |
| 1981 | acatggcatc aggtgggtgg tgatcggaga cgagaactac ggcgagggct cgagccggga |
| 2041 | gcatgcagct ctggagcctc gccaccttgg gggccgggcc atcatcacca agagctttgc |
| 2101 | caggatccac gagaccaacc tgaagaaaca gggcctgctg cctctgacct tcgctgaccc |
| 2161 | ggctgactac aacaagattc accctgtgga caagctgacc attcagggcc tgaaggactt |
| 2221 | cacccctggc aagcccctga agtgcatcat caagcacccc aacgggaccc aggagaccat |
| 2281 | cctcctgaac cacaccttca acgagacgca gattgagtgg ttccgcgctg gcagtgccct |
| 2341 | caacagaatg aaggaactgc aacagtgagg gcagtgcctc cccgccccgc cgctggcgtc |
| 2401 | aagttcagct ccacgtgtgc catcagtgga tccgatccgt ccagccatgg cttcctattc |
| 2461 | caagatggtg tgaccagaca tgcttcctgc tccccgctta gcccacggag tgactgtggt |
| 2521 | tgtggtgggg gggttcttaa aataactttt tagcccccat cttcctattt tgagtttggt |
| 2581 | tcagatctta agcagctcca tgcaactgta tttatttttg atgacaagac tcccatctaa |
| 2641 | agtttttctc ctgcctgatc atttcattgg tggctgaagg attctagaga accttttgtt |
| 2701 | cttgcaagga aaacaagaat ccaaaaccaa aaaaaaaaaa aaaaa |
| 22. IDH1 human DNA, Isocitrate Dehydrogenase (NADP(+)) 1, Cytosolic | |
| (SEQ ID NO: 22) | |
| 1 | ggcggcgaag cgggggcacg ccctcgcaca cgcagagata aattgtgctc ccatgacctt |
| 61 | tatttggaaa gtgcctgcgg gcctaaaatt ggcctttgtc ccaccgagta cactcagcac |
| 121 | tgtactttaa accggataaa ctgggctgtc tggcaggcga taaactacat tcagttgagt |
| 181 | ctgcaagact gggaggaact ggggtgataa gaaatctatt cactgtcaag gtttattgaa |
| 241 | gtcaaaatgt ccaaaaaaat cagtggcggt tctgtggtag agatgcaagg agatgaaatg |
| 301 | acacgaatca tttgggaatt gattaaagag aaactcattt ttccctacgt ggaattggat |
| 361 | ctacatagct atgatttagg catagagaat cgtgatgcca ccaacgacca agtcaccaag |
| 421 | gatgctgcag aagctataaa gaagcataat gttggcgtca aatgtgccac tatcactcct |
| 481 | gatgagaaga gggttgagga gttcaagttg aaacaaatgt ggaaatcacc aaatggcacc |
| 541 | atacgaaata ttctgggtgg cacggtcttc agagaagcca ttatctgcaa aaatatcccc |
| 601 | cggcttgtga gtggatgggt aaaacctatc atcataggtc gtcatgctta tggggatcaa |
| 661 | tacagagcaa ctgattttgt tgttcctggg cctggaaaag tagagataac ctacacacca |
| 721 | agtgacggaa cccaaaaggt gacatacctg gtacataact ttgaagaagg tggtggtgtt |
| 781 | gccatgggga tgtataatca agataagtca attgaagatt ttgcacacag ttccttccaa |
| 841 | atggctctgt ctaagggttg gcctttgtat ctgagcacca aaaacactat tctgaagaaa |
| 901 | tatgatgggc gttttaaaga catctttcag gagatatatg acaagcagta caagtcccag |
| 961 | tttgaagctc aaaagatctg gtatgagcat aggctcatcg acgacatggt ggcccaagct |
| 1021 | atgaaatcag agggaggctt catctgggcc tgtaaaaact atgatggtga cgtgcagtcg |
| 1081 | gactctgtgg cccaagggta tggctctctc ggcatgatga ccagcgtgct ggtttgtcca |
| 1141 | gatggcaaga cagtagaagc agaggctgcc cacgggactg taacccgtca ctaccgcatg |
| 1201 | taccagaaag gacaggagac gtccaccaat cccattgctt ccatttttgc ctggaccaga |
| 1261 | gggttagccc acagagcaaa gcttgataac aataaagagc ttgccttctt tgcaaatgct |
| 1321 | ttggaagaag tctctattga gacaattgag gctggcttca tgaccaagga cttggctgct |
| 1381 | tgcattaaag gtttacccaa tgtgcaacgt tctgactact tgaatacatt tgagttcatg |
| 1441 | gataaacttg gagaaaactt gaagatcaaa ctagctcagg ccaaacttta agttcatacc |
| 1501 | tgagctaaga aggataattg tcttttggta actaggtcta caggtttaca tttttctgtg |
| 1561 | ttacactcaa ggataaaggc aaaatcaatt ttgtaatttg tttagaagcc agagtttatc |
| 1621 | ttttctataa gtttacagcc tttttcttat atatacagtt attgccacct ttgtgaacat |
| 1681 | ggcaagggac ttttttacaa tttttatttt attttctagt accagcctag gaattcggtt |
| 1741 | agtactcatt tgtattcact gtcacttttt ctcatgttct aattataaat gaccaaaatc |
| 1801 | aagattgctc aaaagggtaa atgatagcca cagtattgct ccctaaaata tgcataaagt |
| 1861 | agaaattcac tgccttcccc tcctgtccat gaccttgggc acagggaagt tctggtgtca |
| 1921 | tagatatccc gttttgtgag gtagagctgt gcattaaact tgcacatgac tggaacgaag |
| 1981 | tatgagtgca actcaaatgt gttgaagata ctgcagtcat ttttgtaaag accttgctga |
| 2041 | atgtttccaa tagactaaat actgtttagg ccgcaggaga gtttggaatc cggaataaat |
| 2101 | actacctgga ggtttgtcct ctccattttt ctctttctcc tcctggcctg gcctgaatat |
| 2161 | tatactactc taaatagcat atttcatcca agtgcaataa tgtaagctga atcttttttg |
| 2221 | gacttctgct ggcctgtttt atttctttta tataaatgtg atttctcaga aattgatatt |
| 2281 | aaacactatc ttatcttctc ctgaaaaaaa aaaaaaaaaa aaaaaa |
| 23. IDH2 human DNA, Isocitrate Dehydrogenase (NADP(+)) 2, | |
| Mitochondrial | |
| (SEQ ID NO: 23) | |
| 1 | ggcagccggg aggagcggcg cgcgctcgga cctctcccgc cctgctcgtt cgctctccag |
| 61 | cttgggatgg ccggctacct gcgggtcgtg cgctcgctct gcagagcctc aggctcgcgg |
| 121 | ccggcctggg cgccggcggc cctgacagcc cccacctcgc aagagcagcc gcggcgccac |
| 181 | tatgccgaca aaaggatcaa ggtggcgaag cccgtggtgg agatggatgg tgatgagatg |
| 241 | acccgtatta tctggcagtt catcaaggag aagctcatcc tgccccacgt ggacatccag |
| 301 | ctaaagtatt ttgacctcgg gctcccaaac cgtgaccaga ctgatgacca ggtcaccatt |
| 361 | gactctgcac tggccaccca gaagtacagt gtggctgtca agtgtgccac catcacccct |
| 421 | gatgaggccc gtgtggaaga gttcaagctg aagaagatgt ggaaaagtcc caatggaact |
| 481 | atccggaaca tcctgggggg gactgtcttc cgggagccca tcatctgcaa aaacatccca |
| 541 | cgcctagtcc ctggctggac caagcccatc accattggca ggcacgccca tggcgaccag |
| 601 | tacaaggcca cagactttgt ggcagaccgg gccggcactt tcaaaatggt cttcacccca |
| 661 | aaagatggca gtggtgtcaa ggagtgggaa gtgtacaact tccccgcagg cggcgtgggc |
| 721 | atgggcatgt acaacaccga cgagtccatc tcaggttttg cgcacagctg cttccagtat |
| 781 | gccatccaga agaaatggcc gctgtacatg agcaccaaga acaccatact gaaagcctac |
| 841 | gatgggcgtt tcaaggacat cttccaggag atctttgaca agcactataa gaccgacttc |
| 901 | gacaagaata agatctggta tgagcaccgg ctcattgatg acatggtggc tcaggtcctc |
| 961 | aagtcttcgg gtggctttgt gtgggcctgc aagaactatg acggagatgt gcagtcagac |
| 1021 | atcctggccc agggctttgg ctcccttggc ctgatgacgt ccgtcctggt ctgccctgat |
| 1081 | gggaagacga ttgaggctga ggccgctcat gggaccgtca cccgccacta tcgggagcac |
| 1141 | cagaagggcc ggcccaccag caccaacccc atcgccagca tctttgcctg gacacgtggc |
| 1201 | ctggagcacc gggggaagct ggatgggaac caagacctca tcaggtttgc ccagatgctg |
| 1261 | gagaaggtgt gcgtggagac ggtggagagt ggagccatga ccaaggacct ggcgggctgc |
| 1321 | attcacggcc tcagcaatgt gaagctgaac gagcacttcc tgaacaccac ggacttcctc |
| 1381 | gacaccatca agagcaacct ggacagagcc ctgggcaggc agtaggggga ggcgccaccc |
| 1441 | atggctgcag tggaggggcc agggctgagc cggcgggtcc tcctgagcgc ggcagagggt |
| 1501 | gagcctcaca gcccctctct ggaggccttt ctaggggatg tttttttata agccagatgt |
| 1561 | ttttaaaagc atatgtgtgt ttcccctcat ggtgacgtga ggcaggagca gtgcgtttta |
| 1621 | cctcagccag tcagtatgtt ttgcatactg taatttatat tgcccttgga acacatggtg |
| 1681 | ccatatttag ctactaaaaa gctcttcaca aaaaaaaaaa aaaaaaa |
| 24. IDH3A human DNA, Isocitrate Dehydrogenase 3 (NAD(+)) Alpha | |
| (SEQ ID NO: 24) | |
| 1 | cggagccagg aggggaagcg atggctgggc ccgcgtggat ctctaaggtc tctcggctgc |
| 61 | tgggggcatt ccacaaccca aaacaggtga ccagaggttt tactggtggt gttcagacag |
| 121 | taactttaat tccaggagat ggtattggcc cagaaatttc agctgcagtt atgaagattt |
| 181 | ttgatgctgc caaagcacct attcagtggg aggagcggaa cgtcactgcc attcaaggac |
| 241 | ctggaggaaa gtggatgatc ccttcagagg ctaaagagtc catggataag aacaagatgg |
| 301 | gcttgaaagg ccctttgaag accccaatag cagccggtca cccatctatg aatttactgc |
| 361 | tgcgcaaaac atttgacctt tacgcgaatg tccgaccatg tgtctctatc gaaggctata |
| 421 | aaacccctta caccgatgta aatattgtga ccattcgaga gaacacagaa ggagaataca |
| 481 | gtggaattga gcatgtgatt gttgatggag tcgtgcagag tatcaagctc atcaccgagg |
| 541 | gggcgagcaa gcgcattgct gagtttgcct ttgagtatgc ccggaacaac caccggagca |
| 601 | acgtcacggc ggtgcacaaa gccaacatca tgcggatgtc agatgggctt tttctacaaa |
| 661 | aatgcaggga agttgcagaa agctgtaaag atattaaatt taatgagatg taccttgata |
| 721 | cagtatgttt gaatatggta caagatcctt cccaatttga tgttcttgtt atgccaaatt |
| 781 | tgtatggaga catccttagt gacttgtgtg caggattgat cggaggtctc ggtgtgacac |
| 841 | caagtggcaa cattggagcc aatggggttg caatttttga gtcggttcat gggacggctc |
| 901 | cagacattgc aggcaaggac atggcgaatc ccacagccct cctgctcagt gccgtgatga |
| 961 | tgctgcgcca catgggactt tttgaccatg ctgcaagaat tgaggctgcg tgttttgcta |
| 1021 | caattaagga cggaaagagc ttgacaaaag atttgggagg caatgcaaaa tgctcagact |
| 1081 | tcacagagga aatctgtcgc cgagtaaaag atttagatta acacttctac aactggcatt |
| 1141 | tacatcagtc actctaaatg gacaccacat gaacctctgt ttagaatacc tacgtatgta |
| 1201 | tgcattggtt tgcttgtttc ttgacagtac atttttagat ctggcctttt cttaacaaaa |
| 1261 | tctgtgcaaa agatgcaggt ggatgtccct aggtctgttt tcaaagaact ttttccaagt |
| 1321 | gcttgtttta tttattaagt gtctacctgg taaatgtttt ttttgtaaac tctgagtgga |
| 1381 | ctgtatcatt tgctattcta aaccatttta cacttaagtt aaaatagttt ctcttcagct |
| 1441 | gtaaataaca ggatacagaa ttaacaagag aaaatgtcta actttttaag aaaaacctta |
| 1501 | ttttcttcgg tttttgaaaa acataatgga aataaaacag gatattgaca taatagcaca |
| 1561 | aaatgacact cttctaaaac taaatgggca caagagaatt ttcctgggaa agttcacatc |
| 1621 | aaaaagagtg aatgtggtat atttctaaat gatatggaaa atagagacag atttgtcctt |
| 1681 | tacagaaatt actgagtgtg aataaaaact tcagatccaa gaaatatata atgagagata |
| 1741 | taatttttgt taataagaca aaggtaatat attggataca aagacaaaaa aaaaaaaaaa |
| 1801 | aaa |
| 25. ENO1 human DNA, Enolase 1 | |
| (SEQ ID NO: 25) | |
| 1 | cacggagatc tcgccggctt tacgttcacc tcggtgtctg cagcaccctc cgcttcctct |
| 61 | cctaggcgac gagacccagt ggctagaagt tcaccatgtc tattctcaag atccatgcca |
| 121 | gggagatctt tgactctcgc gggaatccca ctgttgaggt tgatctcttc acctcaaaag |
| 181 | gtctcttcag agctgctgtg cccagtggtg cttcaactgg tatctatgag gccctagagc |
| 241 | tccgggacaa tgataagact cgctatatgg ggaagggtgt ctcaaaggct gttgagcaca |
| 301 | tcaataaaac tattgcgcct gccctggtta gcaagaaact gaacgtcaca gaacaagaga |
| 361 | agattgacaa actgatgatc gagatggatg gaacagaaaa taaatctaag tttggtgcga |
| 421 | acgccattct gggggtgtcc cttgccgtct gcaaagctgg tgccgttgag aagggggtcc |
| 481 | ccctgtaccg ccacatcgct gacttggctg gcaactctga agtcatcctg ccagtcccgg |
| 541 | cgttcaatgt catcaatggc ggttctcatg ctggcaacaa gctggccatg caggagttca |
| 601 | tgatcctccc agtcggtgca gcaaacttca gggaagccat gcgcattgga gcagaggttt |
| 661 | accacaacct gaagaatgtc atcaaggaga aatatgggaa agatgccacc aatgtggggg |
| 721 | atgaaggcgg gtttgctccc aacatcctgg agaataaaga aggcctggag ctgctgaaga |
| 781 | ctgctattgg gaaagctggc tacactgata aggtggtcat cggcatggac gtagcggcct |
| 841 | ccgagttctt caggtctggg aagtatgacc tggacttcaa gtctcccgat gaccccagca |
| 901 | ggtacatctc gcctgaccag ctggctgacc tgtacaagtc cttcatcaag gactacccag |
| 961 | tggtgtctat cgaagatccc tttgaccagg atgactgggg agcttggcag aagttcacag |
| 1021 | ccagtgcagg aatccaggta gtgggggatg atctcacagt gaccaaccca aagaggatcg |
| 1081 | ccaaggccgt gaacgagaag tcctgcaact gcctcctgct caaagtcaac cagattggct |
| 1141 | ccgtgaccga gtctcttcag gcgtgcaagc tggcccaggc caatggttgg ggcgtcatgg |
| 1201 | tgtctcatcg ttcgggggag actgaagata ccttcatcgc tgacctggtt gtggggctgt |
| 1261 | gcactgggca gatcaagact ggtgcccctt gccgatctga gcgcttggcc aagtacaacc |
| 1321 | agctcctcag aattgaagag gagctgggca gcaaggctaa gtttgccggc aggaacttca |
| 1381 | gaaacccctt ggccaagtaa gctgtgggca ggcaagccct tcggtcacct gttggctaca |
| 1441 | cagacccctc ccctcgtgtc agctcaggca gctcgaggcc cccgaccaac acttgcaggg |
| 1501 | gtccctgcta gttagcgccc caccgccgtg gagttcgtac cgcttcctta gaacttctac |
| 1561 | agaagccaag ctccctggag ccctgttggc agctctagct ttgcagtcgt gtaattggcc |
| 1621 | caagtcattg tttttctcgc ctcactttcc accaagtgtc tagagtcatg tgagcctcgt |
| 1681 | gtcatctccg gggtggccac aggctagatc cccggtggtt ttgtgctcaa aataaaaagc |
| 1741 | ctctgtgacc catgaaaaaa aaaaaaaaaa |
| 26. GOT1 human DNA, Glutamic-Oxaloacetic Transaminase 1 | |
| (SEQ ID NO: 26) | |
| 1 | gaaatctctt gattcctagt ctctcgatat ggcacctccg tcagtctttg ccgaggttcc |
| 61 | gcaggcccag cctgtcctgg tcttcaagct cactgccgac ttcagggagg atccggaccc |
| 121 | ccgcaaggtc aacctgggag tgggagcata tcgcacggat gactgccatc cctgggtttt |
| 181 | gccagtagtg aagaaagtgg agcagaagat tgctaatgac aatagcctaa atcacgagta |
| 241 | tctgccaatc ctgggcctgg ctgagttccg gagctgtgct tctcgtcttg cccttgggga |
| 301 | tgacagccca gcactcaagg agaagcgggt aggaggtgtg caatctttgg ggggaacagg |
| 361 | tgcacttcga attggagctg atttcttagc gcgttggtac aatggaacaa acaacaagaa |
| 421 | cacacctgtc tatgtgtcct caccaacctg ggagaatcac aatgctgtgt tttccgctgc |
| 481 | tggttttaaa gacattcggt cctatcgcta ctgggatgca gagaagagag gattggacct |
| 541 | ccagggcttc ctgaatgatc tggagaatgc tcctgagttc tccattgttg tcctccacgc |
| 601 | ctgtgcacac aacccaactg ggattgaccc aactccggag cagtggaagc agattgcttc |
| 661 | tgtcatgaag caccggtttc tgttcccctt ctttgactca gcctatcagg gcttcgcatc |
| 721 | tggaaacctg gagagagatg cctgggccat tcgctatttt gtgtctgaag gcttcgagtt |
| 781 | cttctgtgcc cagtccttct ccaagaactt cgggctctac aatgagagag tcgggaatct |
| 841 | gactgtggtt ggaaaagaac ctgagagcat cctgcaagtc ctttcccaga tggagaagat |
| 901 | cgtgcggatt acttggtcca atccccccgc ccagggagca cgaattgtgg ccagcaccct |
| 961 | ctctaaccct gagctctttg aggaatggac aggtaatgtg aagacaatgg ctgaccggat |
| 1021 | tctgaccatg agatctgaac tcagggcacg actagaagcc ctcaaaaccc ctgggacctg |
| 1081 | gaaccacatc actgatcaaa ttggcatgtt cagcttcact gggttgaacc ccaagcaggt |
| 1141 | tgagtatctg gtcaatgaaa agcacatcta cctgctgcca agtggtcgaa tcaacgtgag |
| 1201 | tggcttaacc accaaaaatc tagattacgt ggccacctcc atccatgaag cagtcaccaa |
| 1261 | aatccagtga agaaacacca cccgtccagt accaccaaag tagttctctg tcatgtgtgt |
| 1321 | tccctgcctg cacaaaccta catgtacata ccatggatta gagacacttg caggactgaa |
| 1381 | aggctgctct ggtgaggcag cctctgttta aaccggcccc acatgaagag aacatccctt |
| 1441 | gagacgaatt tggagactgg gattagagcc tttggaggtc aaagcaaatt aagattttta |
| 1501 | tttaagaata aaagagtact ttgatcatga gaaaaaaaac aaaaaaaaaa aaaaaaaaaa |
| 1561 | aaaaaa |
| 27. GOT2 human DNA, Glutamic-Oxaloacetic Transaminase 2 | |
| (SEQ ID NO: 27) | |
| 1 | gctcgccctc tgctccgtcc tgcggctgcc cactgccctc ctacggtcca ccatggccct |
| 61 | gctgcactcc ggccgcgtcc tccccgggat cgccgccgcc ttccacccgg gcctcgccgc |
| 121 | cgcggcctct gccagagcca gctcctggtg gacccatgtg gaaatgggac ctccagatcc |
| 181 | cattctggga gtcactgaag cctttaagag ggacaccaat agcaaaaaga tgaatctggg |
| 241 | agttggtgcc taccgggatg ataatggaaa gccttacgtt ctgcctagcg tccgcaaggc |
| 301 | agaggcccag attgccgcaa aaaatttgga caaggaatac ctgcccattg ggggactggc |
| 361 | tgaattttgc aaggcatctg cagaactagc cctgggtgag aacagcgaag tcttgaagag |
| 421 | tggccggttt gtcactgtgc agaccatttc tggaactgga gccttaagga tcggagccag |
| 481 | ttttctgcaa agatttttta agttcagccg agatgtcttt ctgcccaaac caacctgggg |
| 541 | aaaccacaca cccatcttca gggatgctgg catgcagcta caaggttatc ggtattatga |
| 601 | ccccaagact tgcggttttg acttcacagg cgctgtggag gatatttcaa aaataccaga |
| 661 | gcagagtgtt cttcttctgc atgcctgcgc ccacaatccc acgggagtgg acccgcgtcc |
| 721 | ggaacagtgg aaggaaatag caacagtggt gaagaaaagg aatctctttg cgttctttga |
| 781 | catggcctac caaggctttg ccagtggtga tggtgataag gatgcctggg ctgtgcgcca |
| 841 | cttcatcgaa cagggcatta atgtttgcct ctgccaatca tatgccaaga acatgggctt |
| 901 | atatggtgag cgtgtaggag ccttcactat ggtctgcaaa gatgcggatg aagccaaaag |
| 961 | ggtagagtca cagttgaaga tcttgatccg tcccatgtat tccaaccctc ccctcaatgg |
| 1021 | ggcccggatt gctgctgcca ttctgaacac cccagatttg cgaaaacaat ggctgcaaga |
| 1081 | agtgaaagtc atggctgacc gcatcattgg catgcggact caactggtct ccaacctcaa |
| 1141 | gaaggagggt tccacccaca attggcaaca catcaccgac caaattggca tgttctgttt |
| 1201 | cacagggcta aagcctgaac aggtggagcg gctgatcaag gagttctcca tctacatgac |
| 1261 | aaaagatggc cgcatctctg tggcaggggt cacctccagc aacgtgggct accttgccca |
| 1321 | tgccattcac caggccacca agtaatgtcc ctggtgcgag gaaacagaga caacctttct |
| 1381 | gtcttcagcc tctgctattg agagcttcac acagacaatg agagagggtg gatggtggtg |
| 1441 | agtggatcat ttctttcagc cacagtgtgt aacactcagc atttgaatgt ttctcagaaa |
| 1501 | agaacatgta gtgacacagg gcagaggcat ccatggctgg cgtctggaat attaaaccaa |
| 1561 | actctccccg gtcctttttt ctccaacttt tctcaaagag tttacatgtg caagaaagtc |
| 1621 | atcgcaccaa aaaacctgtc aattatgcca ttgcaatatt tcagaagctt taactgaagt |
| 1681 | gtcaggttcc tcgtgagaaa cagcacacgt tagaggcttt gagagaaggc ctagttctgt |
| 1741 | catgagtagt cggcctcgtg tctgtcctcc catcttggaa caaccttatc aacaggccgc |
| 1801 | actgcagaaa tgatgtttta tgaaaaccaa tgaggctgct gccactccag caagggaaat |
| 1861 | aatgcagttt cctgtcttat ttaagaaaaa gagaaggctc tcttttctcc cttgtcattg |
| 1921 | ccgttctttt ccttacacgc aaagattttt taactattgc agattttcat cccattctac |
| 1981 | tgcttgattg accatcaact ccatcctatc gagatttatt taagaatgaa gaacataatt |
| 2041 | ttctgctgat gctgtaccct cacccttttc agcaaagaat agtggagagt aggaaactgt |
| 2101 | actttatctc ggcatcctct tgaatgatag tgcaagtttc tccagttggg atgttgtctc |
| 2161 | tgcccggttg gacctcctcc ctttgttgaa tgtggtgtgc agcctctcat ctcacactgt |
| 2221 | gagtccagcg gcgcagggtg gtaccaggaa agaggatatt ctaggctttg cgtgctgcta |
| 2281 | gctgggttca ggcttcaccc actggaaaga accaccatct gctctaacca tgtagactta |
| 2341 | ttgcggcctg gtttctctgt tacaataaaa ttactgtaga cccaaaaaaa aaaaaaaaaa |
| 2401 | aaaaaaaaaa a |
| 28. MUC1 human DNA, Mucin 1, Cell Surface Associated | |
| (SEQ ID NO: 28) | |
| 1 | cgcctgcctg aatctgttct gccccctccc cacccatttc accaccacca tgacaccggg |
| 61 | cacccagtct cctttcttcc tgctgctgct cctcacagtg cttacagcta ccacagcccc |
| 121 | taaacccgca acagttgtta cgggttctgg tcatgcaagc tctaccccag gtggagaaaa |
| 181 | ggagacttcg gctacccaga gaagttcagt gcccagctct actgagaaga atgcttttaa |
| 241 | ttcctctctg gaagatccca gcaccgacta ctaccaagag ctgcagagag acatttctga |
| 301 | aatgtttttg cagatttata aacaaggggg ttttctgggc ctctccaata ttaagttcag |
| 361 | gccaggatct gtggtggtac aattgactct ggccttccga gaaggtacca tcaatgtcca |
| 421 | cgacgtggag acacagttca atcagtataa aacggaagca gcctctcgat ataacctgac |
| 481 | gatctcagac gtcagcgtga gtgatgtgcc atttcctttc tctgcccagt ctggggctgg |
| 541 | ggtgccaggc tggggcatcg cgctgctggt gctggtctgt gttctggttg cgctggccat |
| 601 | tgtctatctc attgccttgg ctgtctgtca gtgccgccga aagaactacg ggcagctgga |
| 661 | catctttcca gcccgggata cctaccatcc tatgagcgag taccccacct accacaccca |
| 721 | tgggcgctat gtgcccccta gcagtaccga tcgtagcccc tatgagaagg tttctgcagg |
| 781 | taatggtggc agcagcctct cttacacaaa cccagcagtg gcagccactt ctgccaactt |
| 841 | gtaggggcac gtcgcccgct gagctgagtg gccagccagt gccattccac tccactcagg |
| 901 | ttcttcaggg ccagagcccc tgcaccctgt ttgggctggt gagctgggag ttcaggtggg |
| 961 | ctgctcacag cctccttcag aggccccacc aatttctcgg aca |
| 29. MCU human DNA, Mitochondrial Calcium Uniporter | |
| (SEQ ID NO: 29) | |
| 1 | ggcggcgttt ccagttgaga gatggcggcc gccgcaggta gatcgctcct gctgctcctc |
| 61 | tcctctcggg gcggcggcgg cgggggcgcc ggcggctgcg gggcgctgac tgccggctgc |
| 121 | ttccctgggc tgggcgtcag ccgccaccgg cagcagcagc accaccggac ggtacaccag |
| 181 | aggatcgctt cctggcagaa tttgggagct gtttattgca gcactgttgt gccctctgat |
| 241 | gatgttacag tggtttatca aaatgggtta cctgtgatat ctgtgaggct accatcccgg |
| 301 | cgtgaacgct gtcagttcac actcaagcct atctctgact ctgttggtgt atttttacga |
| 361 | caactgcaag aagaggatcg gggaattgac agagttgcta tctattcacc agatggtgtt |
| 421 | cgcgttgctg cttcaacagg aatagacctc ctcctccttg atgactttaa gctggtcatt |
| 481 | aatgacttaa cataccacgt acgaccacca aaaagagacc tcttaagtca tgaaaatgca |
| 541 | gcaacgctga atgatgtaaa gacattggtc cagcaactat acaccacact gtgcattgag |
| 601 | cagcaccagt taaacaagga aagggagctt attgaaagac tagaggatct caaagagcag |
| 661 | ctggctcccc tggaaaaggt acgaattgag attagcagaa aagctgagaa gaggaccact |
| 721 | ttggtgctat ggggtggcct tgcctacatg gccacacagt ttggcatttt ggcccggctt |
| 781 | acctggtggg aatattcctg ggacatcatg gagccagtaa catacttcat cacttatgga |
| 841 | agtgccatgg caatgtatgc atattttgta atgacacgcc aggaatatgt ttatccagaa |
| 901 | gccagagaca gacaatactt actatttttc cataaaggag ccaaaaagtc acgttttgac |
| 961 | ctagagaaat acaatcaact caaggatgca attgctcagg cagaaatgga ccttaagaga |
| 1021 | ctgagagacc cattacaagt acatctgcct ctccgacaaa ttggtgaaaa agattgatct |
| 1081 | gcaaaaagcc tctgaatcct ggcagaagga acacctgttt gcctttttaa ttaaagcatt |
| 1141 | gcaggtggaa gctgggagcc atgtgggggg tagagcgttt ttacctttaa ttataaaaca |
| 1201 | aaaacagaaa ggatctgagg gaagaaggga atgttaaaac ctgaggatca ggcattgtgg |
| 1261 | aatataagct caaagggctt agtgaatatt gtcttaacca agtatctcag tttctggatg |
| 1321 | aaaatgatgc agttatatag ttgagagatt cataaagaga aaacaatgct gggggtgttc |
| 1381 | gtttcttgca tcttctttgc agagtcagca aaagagtaac acaccagcac cccactcgac |
| 1441 | tctatttgtt tttaatttaa ctgtccctat ttttgacata ggagtaaata aatatactag |
| 1501 | aaaagcaaat tctcatgata tgctaaaata tcattagcat ttattttaaa ttggacccag |
| 1561 | tctctgcaga gttaccagga atctttcctt ccagcatccc tttactgtcc acctacctgt |
| 1621 | acctcttggt tacactcatt ttttccattt gataattgga accaacttat aactgtttaa |
| 1681 | taattgacac tttagattat ctcttaatac cttcttaaat gtctatatat cccagtgctc |
| 1741 | tggatcagtg tctaaaaatc actggcaaca ctgcatgagg ttgttggttt tgttttgttt |
| 1801 | tattaattag tctttcacag gaggaataat tgccctcctt tatatactta tctattgata |
| 1861 | atcccctctc cctccagaac acaaatcaga gggaaagggg gtgttcagct gtactaccaa |
| 1921 | atcaggaaga tgtaaggttt acaaattggc taagaatcat ggctctgtag ccatttcaac |
| 1981 | cagaataatt ttattgctaa tctgctttgt gtgacagcat tccaggccag ccagatggga |
| 2041 | ctgccttgtc tggaggcttt gttcatctcg aaggacacac acttccacac tgtttgtgag |
| 2101 | ccctcccacc tccacaactt cagttgtaaa tcaagtgtgt ggatctcaaa gggtgcaatt |
| 2161 | tatctttata taggaataca tttctagggc ttccttcaag cccactctct tcaccctatt |
| 2221 | ttttcttatc ttaaattgag agaaagagaa ttaatcttat actttgtcaa aacattttct |
| 2281 | accatatttc cagatgacat ctgcgcttga agagtcaaag gaatctgtgt ctaatatcct |
| 2341 | gtttttaact gctgtagggg caggatggaa aggatgatgg gggctgccac accactgatt |
| 2401 | ggccttttct ttcacgtgat tcatccttcc tcattgtggc aaggagtttc tttctctttt |
| 2461 | tcttcctcct ttgggatcat tgtgtatgaa aagaaaaact ttaaatgaca aacccagact |
| 2521 | ccaggtgcct tgcaaaggtt gaaggccagc caggattgct gctgctgctg ctactcctgc |
| 2581 | caacacccct ttcattggca tgacggaatg aaaggatgca tgtctccact tcctgaccct |
| 2641 | ccgcccactt ccttctccct ccaccacccc cagtcgtcag ctccttccct catttatttt |
| 2701 | tgttaagttg tgtgaattat ttttaaccca tttatcctgt ttgtgcatag ggtttttaag |
| 2761 | aagaaacagc acagtgcaac gagcaaatct ttttggggtg tgtgggaagc aagggaggga |
| 2821 | ggacatggag aaaagttctt taaacaaata gcaaactatt gaacatgtgt aaaatcctgt |
| 2881 | atcatttatg aaatatgtat aaaaagcaat gtaccttctg gaacaataaa tacttattca |
| 2941 | atttttgaaa aaaaaaaaaa aaaaaaaaaa aaaaaaaa |
| 30. AAV2 ITR 5′ ITR | |
| (SEQ ID NO: 30) | |
| CCTGCAGGCAGCTGCGCGCTCGCTCGCTCACTGAGGCCGCCCGGGCGTCGGGCG | |
| ACCTTTGGTCGCCCGGCCTCAGTGAGCGAGCGAGCGCGCAGAGAGGGAGTGGCC | |
| AACTCCATCACTAGGGGTTCCT | |
| 31. AAV2 ITR 3′ ITR | |
| (SEQ ID NO: 31) | |
| CCTGCAGGCAGCTGCGCGCTCGCTCGCTCACTGAGGCCGCCCGGGCAAAGCCCG | |
| GGCGTCGGGCGACCTTTGGTCGCCCGGCCTCAGTGAGCGAGCGAGCGCGCAGAG | |
| AGGGAGTGGCCAACTCCATCACTAGGGGTTCCT | |
| 32. AAV2 vector backbone; bold italicized regions | |
| represent ligation overhangs | |
| (SEQ ID NO: 32) | |
| [symbol] AATCAACCTCTGGATTACAAAATTTGTGAAAGATTGACTGGTATTCTTAA | |
| CTATGTTGCTCCTTTTACGCTATGTGGATACGCTGCTTTAATGCCTTTGTATCAT | |
| GCTATTGCTTCCCGTATGGCTTTCATTTTCTCCTCCTTGTATAAATCCTGGTTAG | |
| TTCTTGCCACGGCGGAACTCATCGCCGCCTGCCTTGCCCGCTGCTGGACAGGGGC | |
| TCGGCTGTTGGGCACTGACAATTCCGTGGTGTTTATTTGTGAAATTTGTGATGCT | |
| ATTGCTTTATTTGTAACCATTCTAGCTTTATTTGTGAAATTTGTGATGCTATTGC | |
| TTTATTTGTAACCATTATAAGCTGCAATAAACAAGTTAACAACAACAATTGCATT | |
| CATTTTATGTTTCAGGTTCAGGGGGAGATGTGGGAGGTTTTTTAAAGCGGGGGAT | |
| CCAAATTCCCGATAAGGATCTTCCTAGAGCATGGCTACGTAGATAAGTAGCATGG | |
| CGGGTTAATCATTAACTACAAGGAACCCCTAGTGATGGAGTTGGCCACTCCCTCT | |
| CTGCGCGCTCGCTCGCTCACTGAGGCCGGGCGACCAAAGGTCGCCCGACGCCCGG | |
| GCTTTGCCCGGGCGGCCTCAGTGAGCGAGCGAGCGCGCAGCCTTAATTAACCTAA | |
| TTCACTGGCCGTCGTTTTACAACGTCGTGACTGGGAAAACCCTGGCGTTACCCAA | |
| CTTAATCGCCTTGCAGCACATCCCCCTTTCGCCAGCTGGCGTAATAGCGAAGAGG | |
| CCCGCACCGATCGCCCTTCCCAACAGTTGCGCAGCCTGAATGGCGAATGGGACGC | |
| GCCCTGTAGCGGCGCATTAAGCGCGGCGGGTGTGGTGGTTACGCGCAGCGTGACC | |
| GCTACACTTGCCAGCGCCCTAGCGCCCGCTCCTTTCGCTTTCTTCCCTTCCTTTC | |
| TCGCCACGTTCGCCGGCTTTCCCCGTCAAGCTCTAAATCGGGGGCTCCCTTTAGG | |
| GTTCCGATTTAGTGCTTTACGGCACCTCGACCCCAAAAAACTTGATTAGGGTGAT | |
| GGTTCACGTAGTGGGCCATCGCCCTGATAGACGGTTTTTCGCCCTTTGACGTTGG | |
| AGTCCACGTTCTTTAATAGTGGACTCTTGTTCCAAACTGGAACAACACTCAACCC | |
| TATCTCGGTCTATTCTTTTGATTTATAAGGGATTTTGCCGATTTCGGCCTATTGG | |
| TTAAAAAATGAGCTGATTTAACAAAAATTTAACGCGAATTTTAACAAAATATTAA | |
| CGTTTATAATTTCAGGTGGCATCTTTCGGGGAAATGTGCGCGGAACCCCTATTTG | |
| TTTATTTTTCTAAATACATTCAAATATGTATCCGCTCATGAGACAATAACCCTGA | |
| TAAATGCTTCAATAATATTGAAAAAGGAAGAGTATGAGTATTCAACATTTCCGTG | |
| TCGCCCTTATTCCCTTTTTTGCGGCATTTTGCCTTCCTGTTTTTGCTCACCCAGA | |
| AACGCTGGTGAAAGTAAAAGATGCTGAAGATCAGTTGGGTGCACGAGTGGGTTAC | |
| ATCGAACTGGATCTCAATAGTGGTAAGATCCTTGAGAGTTTTCGCCCCGAAGAAC | |
| GTTTTCCAATGATGAGCACTTTTAAAGTTCTGCTATGTGGCGCGGTATTATCCCG | |
| TATTGACGCCGGGCAAGAGCAACTCGGTCGCCGCATACACTATTCTCAGAATGAC | |
| TTGGTTGAGTACTCACCAGTCACAGAAAAGCATCTTACGGATGGCATGACAGTAA | |
| GAGAATTATGCAGTGCTGCCATAACCATGAGTGATAACACTGCGGCCAACTTACT | |
| TCTGACAACGATCGGAGGACCGAAGGAGCTAACCGCTTTTTTGCACAACATGGGG | |
| GATCATGTAACTCGCCTTGATCGTTGGGAACCGGAGCTGAATGAAGCCATACCAA | |
| ACGACGAGCGTGACACCACGATGCCTGTAGTAATGGTAACAACGTTGCGCAAACT | |
| ATTAACTGGCGAACTACTTACTCTAGCTTCCCGGCAACAATTAATAGACTGGATG | |
| GAGGCGGATAAAGTTGCAGGACCACTTCTGCGCTCGGCCCTTCCGGCTGGCTGGT | |
| TTATTGCTGATAAATCTGGAGCCGGTGAGCGTGGGTCTCGCGGTATCATTGCAGC | |
| ACTGGGGCCAGATGGTAAGCCCTCCCGTATCGTAGTTATCTACACGACGGGGAGT | |
| CAGGCAACTATGGATGAACGAAATAGACAGATCGCTGAGATAGGTGCCTCACTGA | |
| TTAAGCATTGGTAACTGTCAGACCAAGTTTACTCATATATACTTTAGATTGATTT | |
| AAAACTTCATTTTTAATTTAAAAGGATCTAGGTGAAGATCCTTTTTGATAATCTC | |
| ATGACCAAAATCCCTTAACGTGAGTTTTCGTTCCACTGAGCGTCAGACCCCGTAG | |
| AAAAGATCAAAGGATCTTCTTGAGATCCTTTTTTTCTGCGCGTAATCTGCTGCTT | |
| GCAAACAAAAAAACCACCGCTACCAGCGGTGGTTTGTTTGCCGGATCAAGAGCTA | |
| CCAACTCTTTTTCCGAAGGTAACTGGCTTCAGCAGAGCGCAGATACCAAATACTG | |
| TCCTTCTAGTGTAGCCGTAGTTAGGCCACCACTTCAAGAACTCTGTAGCACCGCC | |
| TACATACCTCGCTCTGCTAATCCTGTTACCAGTGGCTGCTGCCAGTGGCGATAAG | |
| TCGTGTCTTACCGGGTTGGACTCAAGACGATAGTTACCGGATAAGGCGCAGCGGT | |
| CGGGCTGAACGGGGGGTTCGTGCACACAGCCCAGCTTGGAGCGAACGACCTACAC | |
| CGAACTGAGATACCTACAGCGTGAGCTATGAGAAAGCGCCACGCTTCCCGAAGGG | |
| AGAAAGGCGGACAGGTATCCGGTAAGCGGCAGGGTCGGAACAGGAGAGCGCACGA | |
| GGGAGCTTCCAGGGGGAAACGCCTGGTATCTTTATAGTCCTGTCGGGTTTCGCCA | |
| CCTCTGACTTGAGCGTCGATTTTTGTGATGCTCGTCAGGGGGGCGGAGCCTATGG | |
| AAAAACGCCAGCAACGCGGCCTTTTTACGGTTCCTGGCCTTTTGCTGGCCTTTTG | |
| CTCACATGTTCTTTCCTGCGTTATCCCCTGATTCTGTGGATAACCGTATTACCGC | |
| CTTTGAGTGAGCTGATACCGCTCGCCGCAGCCGAACGACCGAGCGCAGCGAGTCA | |
| GTGAGCGAGGAAGCGGAAGAGCGCCCAATACGCAAACCGCCTCTCCCCGCGCGTT | |
| GGCCGATTCATTAATGCAGCTGGCACGACAGGTTTCCCGACTGGAAAGCGGGCAG | |
| TGAGCGCAACGCAATTAATGTGAGTTAGCTCACTCATTAGGCACCCCAGGCTTTA | |
| CACTTTATGCTTCCGGCTCGTATGTTGTGTGGAATTGTGAGCGGATAACAATTTC | |
| ACACAGGAAACAGCTATGACCATGATTACGCCAGATTTAATTAAGGCCTTAATTA | |
| GGCTGCGCGCTCGCTCGCTCACTGAGGCCGCCCGGGCAAAGCCCGGGCGTCGGGC | |
| GACCTTTGGTCGCCCGGCCTCAGTGAGCGAGCGAGCGCGCAGAGAGGGAGTGGCC | |
| AACTCCATCACTAGGGGTTCCTTGTAGTTAATGATTAACCCGCCATGCTACTTAT | |
| CTACGTAGCCATGCTCTAGGAAGATCGGAATTCCTAGGCTCCGGTGCCCGTCAGT | |
| GGGCAGAGCGCACATCGCCCACAGTCCCCGAGAAGTTGGGGGGAGGGGTCGGCAA | |
| TTGAACCGGTGCCTAGAGAAGGTGGCGCGGGGTAAACTGGGAAAGTGATGTCGTG | |
| TACTGGCTCCGCCTTTTTCCCGAGGGTGGGGGAGAACCGTATATAAGTGCAGTAG | |
| TCGCCGTGAACGTTCTTTTTCGCAACGGGTTTGCCGCCAGAACACA [symbol] | |
| 33. Forward Primer used to amplify COL3A1 where bold | |
| italisized region represents the Kozac sequence | |
| (SEQ ID NO: 33) | |
| ATGTTAGCGGCCGC [symbol] ATGATGAGCTTTGTGCAAAAGGGGAGC | |
| 34. Reverse Primer used to amplify COL3A1, where bold | |
| italisized region represents a stop codon | |
| (SEQ ID NO: 34) | |
| CTTACGGCTAGC [symbol] TTATAAAAAGCAAACAGGGCCAACGTCCAC | |
| 35. ACTB human DNA, Actin Beta | |
| (SEQ ID NO: 35) | |
| 1 | gttcgttgca acaaattgat gagcaatgct tttttataat gccaactttg tacaaaaaag |
| 61 | ttggcatgga tgatgatatc gccgcgctcg tcgtcgacaa cggctccggc atgtgcaagg |
| 121 | ccggcttcgc gggcgacgat gccccccggg ccgtcttccc ctccatcgtg gggcgcccca |
| 181 | ggcaccaggg cgtgatggtg ggcatgggtc agaaggattc ctatgtgggc gacgaggccc |
| 241 | agagcaagag aggcatcctc accctgaagt accccatcga gcacggcatc gtcaccaact |
| 301 | gggacgacat ggagaaaatc tggcaccaca ccttctacaa tgagctgcgt gtgcctcccg |
| 361 | aggagcaccc cgtgctgctg accgaggccc ccctgaaccc caaggccaac cgcgagaaga |
| 421 | tgacccagat catgtttgag accttcaaca ccccagccat gtacgttgct atccaggctg |
| 481 | tgctatccct gtacgcctct ggccgtacca ctggcatcgt gatggactcc ggtgacgggg |
| 541 | tcacccacac tgtgcccatc tacgaggggt atgccctccc ccatgccatc ctgcgtctgg |
| 601 | acctggctgg ccgggacctg actgactacc tcatgaagat cctcaccgag cgcggctaca |
| 661 | gcttcaccac cacggccgag cgggaaatcg tgcgtgacat taaggagaag ctgtgctacg |
| 721 | tcgccctgga cttcgagcaa gagatggcca cggctgcttc cagctcctcc ctggagaaga |
| 781 | gctacgagct gcctgacggc caggtcatca ccattggcaa tgagcggttc cgctgccctg |
| 841 | aggcactctt ccagccttcc ttcctgggca tggagtcctg tggcatccac gaaactacct |
| 901 | tcaactccat catgaagtgt gacgtggaca tccgcaaaga cctgtacgcc aacacagtgc |
| 961 | tgtctggcgg caccaccatg taccctggca ttgccgacag gatgcagaag gagatcactg |
| 1021 | ccctggcacc cagcacaatg aagatcaaga tcattgctcc tcctgagcgc aagtactccg |
| 1081 | tgtggatcgg cggctccatc ctggcctcgc tgtccacctt ccagcagatg tggatcagca |
| 1141 | agcaggagta tgacaagtcc ggcccctcca tcgtccaccg caaatgcttc tacccaactt |
| 1201 | tcttgtacaa agttggcatt ataagaaagc attgcttatc aatttgttgc aacgaac |

### Example III

### Identification of Biomarkers for Skin Aging

Chronic exposure to UV irradiation causes an aged phenotype (photo-aging) that is superimposed with chronological aging of the skin. As a consequence, nearly every aspect of skin biology is affected by aging. The self-renewing capability of the epidermis, which provides vital barrier function, is diminished with age and results in numerous clinical presentations, ranging from benign but potentially excruciating disorders like pruritus and defective wound healing to the more threatening carcinomas and melanomas. Yet our current knowledge of the molecular determinants of declining epidermal function in the elderly population is quite limited. Several genome-wide studies have attempted to analyze the transcriptome and epigenome of the aging skin but failed to identify robust drivers of cellular aging in the skin epidermis (Haustead, D. J., Stevenson, A., Saxena, V., Marriage, F., Firth, M., Silla, R., Martin, L., Adcroft, K. F., Rea, S., Day, P. J., Melton, P., Wood, F. M. & Fear, M. W. Transcriptome analysis of human ageing in male skin shows mid-life period of variability and central role of NF-kappaB. Sci Rep 6, 26846 (2016); Makrantonaki, E., Brink, T. C., Zampeli, V., Elewa, R. M., Mlody, B., Hossini, A. M., Hermes, B., Krause, U., Knolle, J., Abdallah, M., Adjaye, J. & Zouboulis, C. C. Identification of biomarkers of human skin ageing in both genders. Wnt signalling—a label of skin ageing? PLoS One 7, e50393 (2012); Raddatz, G., Hagemann, S., Aran, D., Sohle, J., Kulkarni, P. P., Kaderali, L., Hellman, A., Winnefeld, M. & Lyko, F. Aging is associated with highly defined epigenetic changes in the human epidermis. Epigenetics Chromatin 6, 36 (2013)), and reported that the mammalian epidermis appears to resist the aging process (Racila, D. & Bickenbach, J. R. Are epidermal stem cells unique with respect to aging? Aging (Albany N.Y.) 1, 746-50 (2009)). It is widely accepted that besides transcriptional and epigenetic changes, cellular aging is characterized also by profound metabolic alterations. Nonetheless, recent non-targeted metabolomics analysis of full thickness human skin indicated that only a minimal fraction (less than 10%) of detectable metabolites significantly drifted during aging [Kuehne, A., Hildebrand, J., Soehle, J., Wenck, H., Terstegen, L., Gallinat, S., Knott, A., Winnefeld, M. & Zamboni, N. An integrative metabolomics and transcriptomics study to identify metabolic alterations in aged skin of humans in vivo. BMC Genomics 18, 169 (2017); Randhawa, M., Sangar, V., Tucker-Samaras, S. & Southall, M. Metabolic signature of sun exposed skin suggests catabolic pathway overweighs anabolic pathway. PLoS One 9, e90367 (2014)). To the best of our knowledge, all reports on omics studies of transcriptome, epigenome, and metabolome of aging human skin use bulk analysis performed on whole tissue lysates, which very often fails to detect profound changes in isolated small cellular populations (such as stem cells) which drive homeostatic processes.

Adult organs are maintained through a balance of proliferation, differentiation, and self-renewal of stem cells that take place during normal tissue homeostasis or tissue repair. The epidermis relies on a population of stem cells and proliferating progenitors to continuously maintain its barrier-protective function. It is composed of different cellular lineages: the interfollicular epidermis and its appendages; and the sebaceous glands and the hair follicles. Human epidermis is regenerated approximately every 4 weeks, a process driven by commitment of progenitor cells located within the basal membrane which develop into more differentiated populations. Initial models of epidermal maintenance proposed that the basal layer is composed of two populations of stem cells: slow cycling stem cells and their transiently amplifying progenitors. However, recent advances in linage tracing and live imaging techniques combined with genetic manipulations have now established a simple model of epidermal homeostasis in which basal keratinocytes are born as equally uncommitted stem cells making random choices to divide or differentiate. This process allows both for continuous renewal of the proliferating basal layer and departure of committed cells away from the basal membrane towards the differentiated upper layers of the epidermis. The ultimate goal of this homeostatic behavior is the generation of a solid cornified envelope as a barrier to the outside insults.

### Mass Spectroscopy (LC-MS/MS)

primary cultures of human keratinocytes from donors of different ages (ranging from Age 18 to Age 72) WERE isolated and grown in strictly progenitor conditions as described in (Roshan, A., Murai, K., Fowler, J., Simons, B. D., Nikolaidou-Neokosmidou, V. & Jones, P. H. Human keratinocytes have two interconvertible modes of proliferation. Nat Cell Biol 18, 145-56 (2016)), after which half of the population were prompted to commit to differentiation by exogenous calcium. To investigate the metabolic changes occurring during the process of differentiation, we subjected both populations (progenitors and committed cells) from young, medium, and old ages to polar steady-state metabolomics analysis by liquid chromatography-based tandem mass spectrometry (LC-MS/MS), and determined 296 metabolite profiles for each sample. Experiments with progenitor and committed primary cultures were run in triplicates and values for every measured metabolite were compared across all samples. Normalization was performed based on cell number in each individual sample. Alterations in multiple classes of metabolites were observed by hierarchical clustering in the mature keratinocyte population (but not the progenitor population) pointing to age-related functional metabolic deteriorations of progenitors' ability to build a young epidermis.

### Whole Transcriptome RNA Sequencing

For each sample, total RNA was extracted using RNeasy mini kit (Qiagen) and treated with on-column RNase-free DNase I (Qiagen) following manufacturer's instructions. 1 ug of RNA from each sample was used for library preparation. RNA-seq libraries were constructed using TruSeq Stranded Total RNA Library Prep Kit with Ribo-Zero Gold (Illumina) designed for cytoplasmic and mitochondrial rRNA depletion. All coding RNA and certain forms of non-coding RNA were isolated using bead-based rRNA depletion, followed by cDNA synthesis, and PCR amplification as per manufacturer's protocol. Final libraries were analyzed on Bioanalyzer (Agilent), quantified with qPCR, pooled together, and run on one lane of an Illumina HiSeq 2500 using 2×100-bp paired-end reads. The Illumina paired-end adapter sequences were removed from the raw reads using Cutadapt v1.8.1. The TruSeq adaptor sequence 5′-AGATCGGAAGAGCACACGTCTGAACTCCAGTCAC-3′ was used for read 1, and its reverse complement, 3′-AGATCGGAAGAGCGTCGTGTAGGGAAAGAGTGTAGATCTCGGTGGTCGCCGTATCATT-5′ was used for read 2.

Next, RNA libraries were processed using a pipeline which includes STAR-HtSeq-GFOLD for alignment, count generation, and gene expression. Briefly, STAR aligner (v. 2.4.0j) was used to map the reads to hg19, and HtSeq was used to generate gene expression counts. Since each donor is considered an N of 1 (i.e. donors are not grouped in replicates), GFOLD and custom R scripts were used to determine gene and differential expression.

### Biological Network Propagation

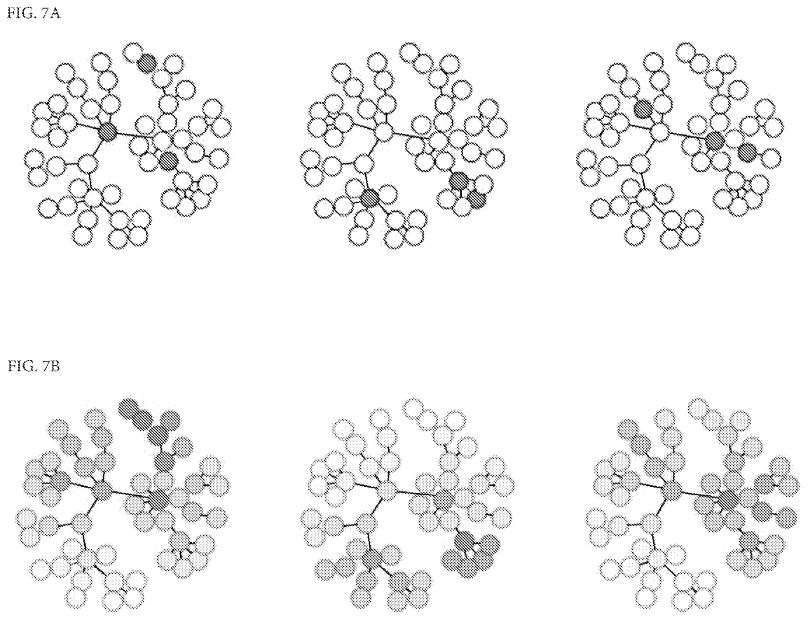

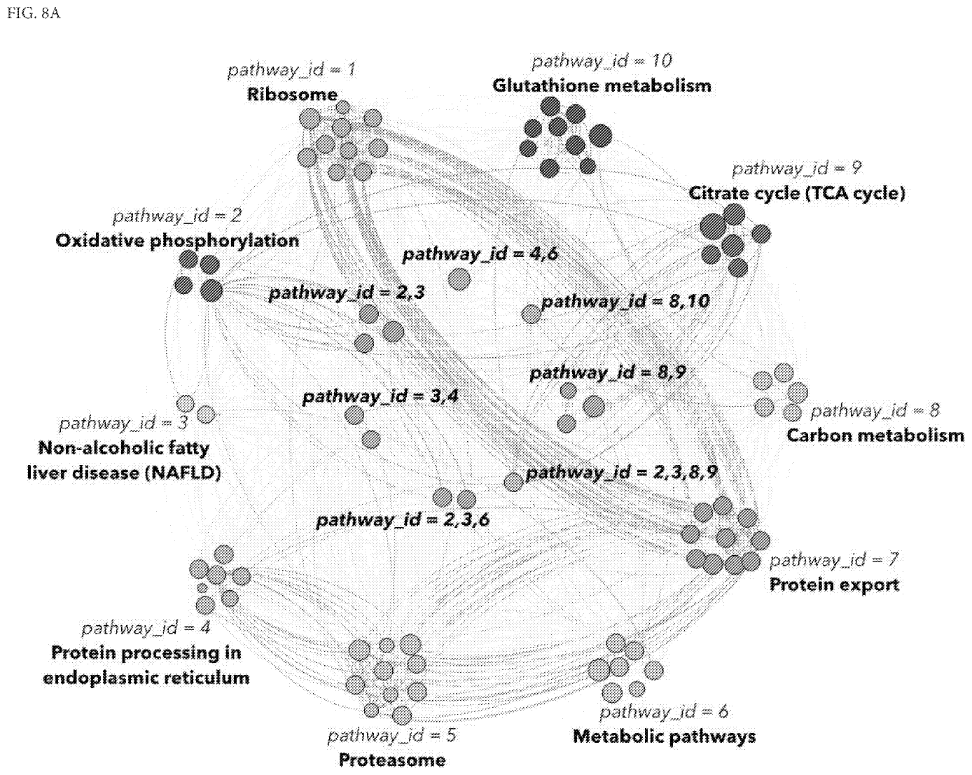

To identify functional modules of genes that reflect age-related changes, gene-gene interaction (protein-protein interaction, PPI) network were integrated with expression data by a computational algorithm. This method is called Network Propagation, which propagates the expression values by the topology of the network. In literature, network propagation is typically employed on mutation data to classify cancer informative subtypes by clustering patients with mutations in similar network regions (Vanunu O, Magger O, Ruppin E, Shlomi T, Sharan R (2010) Associating genes and protein complexes with disease via network propagation. PLoS Comput Biol 6: e1000641). In this analysis, a network graph was first created by using the STRING database, such that nodes correspond to genes and edges correspond to interactions between genes. For each gene (node), the value was mapped with the deviation of gene expression in specific sample (age18, age46, age64, or age72) and the mean value of the four samples. Next, the implementation of network propagation processed a random work on a network with the function: Fₜ₊₁=αFₜA+(1−α)F₀, where F₀ is a comparison-by-gene matrix, A is a degree-normalized adjacency matrix derived from the topology of the network, α is a tuning parameter governing the amount of signal that was passed to the neighboring nodes of the network during signal propagation. Based on the function, the propagation occurred by iteration during which a certain ratio of the node value was spread to its neighbors. After several iterations, each gene gets a propagated score. Genes with high propagated scores were regarded as candidates associated with aging functions. In different age samples, a common module with higher propagated values (1,306 genes) was detected by clustering the results from the propagation. FIGS. 7A-7B show an illustration of the network propagation method, FIG. 7A shows three identical networks before network propagation with three different nodes were assigned with values, while FIG. 7B demonstrates the network after propagation. Here higher brightness of a node responds to a higher score.

### O2-PLS Method for Integrative Modeling of Transcript and Metabolite Data

Next a gene set highly correlated with metabolites across different ages was identified using the O2-PLS regression method which is based on partial least squares and orthogonal signal correction (OSC) filter (Johan Trygg, Svante Wold, O2-PLS, a two-block (X-Y) latent variable regression (LVR) method with integral OSC filter, J. Chemometrics; 17: 53-64 (2003)). Broadly, the algorithm separates structured noise from transcript and metabolite matrices, X and Y respectively, and identifies a joint covariation to use in a predictive model. To identify highly correlated genetic and metabolic markers of aging, we used the model on metabolic data of progenitor and committed to differentiation primary cells, and transcriptomic data on progenitor cells only. As a measure of stem cell function, i.e. capacity to commit to differentiation and form proper epidermis, we calculated a metabolic score matrix based on the difference of metabolite levels in the differentiated cell population relative to that of their progenitors for ages ranging from young to old. We then combined these metabolic scores with the transcript data across different ages to identify trends of common variation. Briefly, the algorithm consists of: 1. Decomposition of the covariance Y T X matrix into orthogonal score matrix C, singular value matrix D, and orthonormal loading matrix W; 2. Calculation of X score matrix T where T=XW, and respective removal of structured noise; 3. Calculation of Y score matrix U where U=YC, and respective removal structured noise; and 4. Predictions of U and T with least squares. We determined significance level of (1−α), with α=0.05/n for the transcript data with n=number of genes, and α=0.05/m for the metabolite data with m=number of metabolites. We then performed randomization by reshuffling the original data sets X and Y, 1000 times, and identified thresholds for lower and upper α/2 quantiles for transcript and metabolite correlation loadings. We determined a list of 176 significant genes which we included in network propagation for further processing as part of the network.

### Distance Network

Global network-similarity measures were adopted to better capitalize on biological relationships between selected genes and identify master regulators of skin stem and progenitor cells aging. A gene association network was first constructed by mapping STRING network to each gene level. The network is an undirected graph in which nodes represent genes and edges between two nodes denote an association between two corresponding genes. Weights are used on the edges to represent the probability that such an association exists. After constructing the gene network graph, a random walk graph kernel method was used to capture global relationships within the graph. A graph kernel is a kernel function that computes the probability of reaching one node after a random walk starting from another node, and the computed probability is used for global similarity of two nodes (genes). The resulting graph creates a global distance network where the edge between two nodes (genes) represents the global distance in this network instead of a direct interaction. Laplacian Exponential Diffusion Kernel was used as the kernel function, i.e.:

$$K = {lim}_{n \to ∞} {( I + \frac{β L}{n} )}^{n} = {e}^{β L} ,$$

where L is an undirected graph Laplacian matrix, and β is the diffusion parameter that determines the degree of diffusion. e^{βL} is a random walk that starts from a node to its neighboring node with the probability β. Since e^{βL} is positive definite for a Laplacian matrix, it can be used as a kernel matrix. The resulting kernel matrix is a connected network, which detected not only the direct interaction from the original gene association network, but also all indirect interactions via other genes. As a result, the distances among all genes in the network are determined. Next, these distances are used to distinguish highly expressed neighborhoods with a certain distance from a candidate gene, even in cases when the genes may not directly interact. However, the above equation cannot be solved directly because of computation complexity (O(n³)). We therefore further applied a dimension reduction method, called Cholesky decomposition, to trim the Laplacian matrix L by decomposing it into the product of a lower triangular matrix. Cholesky decomposition is to transform a matrix A into a product of a lower triangular matrix P of rank n (P=(pᵢⱼ) with pᵢⱼ=0 if i<j and pᵢⱼ>0, where i=1, 2, . . . , n) and its transpose, P^{T}: A=P·P^{T}. To reduce the dimensionalities of kernel matrices, we applied the Incomplete Cholesky Decomposition (ICD) with pivoting in order to reduce the dimensionalities by approximating a lower rank matrix (m<<n), such that A≈\tilde{P}·\tilde{P}^{T}, where A∈R^{n×m}, m<<n. We then obtained a lower triangular matrix \tilde{P} of rank m. The overall complexity was O(m²n) and the storage requirement was O(mn). Previously, similar approach has been implemented by Nitch et. al in C++ for the study on disease-causing genes in monogenic genetic diseases [Nitsch D, Tranchevent L C, Thienpont B, Thorrez L, Van Esch H, et al. (2009) Network analysis of differential expression for the identification of disease-causing genes. PLoS One 4: e5526.]. In our study, we constructed the network in R environment.

### Scoring the Genes by Integrating the Distance Network and Gene Expression

The gene expression profiles were mapped to the distance network obtained above. More specifically, the fold changes between the two conditions (two ages) were computed to obtain the differential expression level for the genes in genome. It was considered whether the gene was highly differentially expressed or not, hence, the absolute value of the fold-change was relevant for our method. All differential expression levels, without threshold to distinguish between highly and lowly differentially expressed genes, were used to compute the scores. Since our method computes the scores with all differential expression, there is no threshold used to distinguish between highly and lowly differentially expressed genes. The score of the candidate biomarker gene was calculated by measuring the differential expression levels of its neighborhood. First, the differential expression level of all neighbors in the distance network were ordered by their distance to the candidate gene. The rank of the diffusion distance was then taken as the new distance measure. Second, the new differential expression levels were generated by multiplying the gene expression (fold change value here) with a weighting function (w=e^{-β≠γ}, where γ is the rank, β is the parameter of neighborhood size) to consider the expression of both close and far neighbors. β is the scale parameter to determine how quickly the weight decreased as a function of distance, and it was set to 0.5 to reach sufficiently far away genes in the network for the candidate gene. Lastly, we randomly shuffled original expression values over the network, and then defined the gene score for a candidate gene was by select the maximum deviation between the new differential expression values (weighted) and the randomized expression. Hence, the gene score was related to the level of differential expression level of close neighbors. To estimate the significance of the signal of the actual candidates, we defined the distribution of the scores by randomly distributing the expression data on the network and repeating 3,000 times. By comparing the score of each candidate gene, an empirical p-value for each candidate gene was determined. The score of a candidate gene was considered significant if the score was greater than 95% (α=0.05).

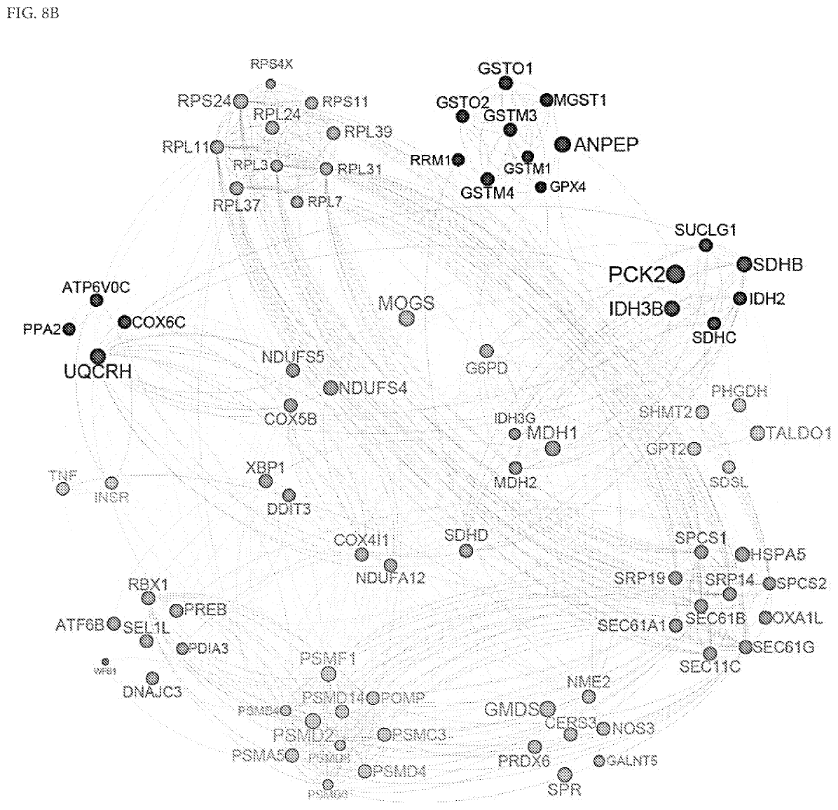

Although previous analyses of aging human skin revealed only non-significant changes in transcriptome, epigenome and metabolome and failed to define molecular drivers of altered epidermal function in the elderly, our model for selection of aging biomarker genes indicates that the processes associated with skin aging hinder the ability of epidermal progenitors to effectively differentiate to mature keratinocytes during commitment to differentiation, resulting in thinner aged epidermis. In FIG. 8A, a network was proposed built upon the top 10 most significantly enriched (non-disease) KEGG pathways of our analysis with an FDR q-value<0.01, and report the top genes with highest scoring (as generated by integrating the distant network and gene expression of the neighborhood) involved in one or multiple of the enriched top pathways (FIG. 8B). The node sizes in the network represent log 2 fold change of gene expression from young to old, i.e. negative values represent a decrease, while positive values—an increase. The strength of gene-to-gene interactions is visualized by light to dark hue, i.e. from weak to strong, respectively. Finally, top hits scored by weighted gene expression of their distant network are reported as master regulators in the following pathways: 1_Ribosome, 2_Oxidative phosphorylation, 3_Non-alcoholic fatty liver disease (NAFLD), 4_Protein processing in endoplasmic reticulum, 5_Proteasome, 6_Metabolic pathways, 7_Protein export, 8_Carbon metabolism, 9_Citrate cycle (TCA cycle), 10_Glutathione metabolism. The visualization results were summarized from FIG. 8 in TABLE 3.

### Validation of Maleate Dehydrogenese 2 (MDH2)

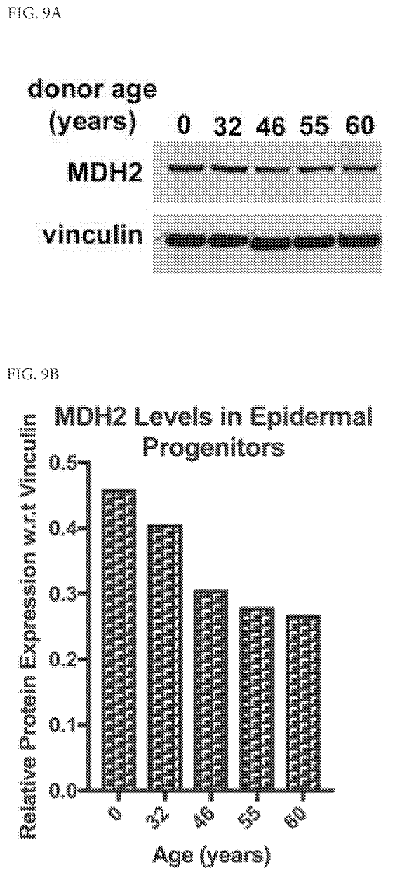

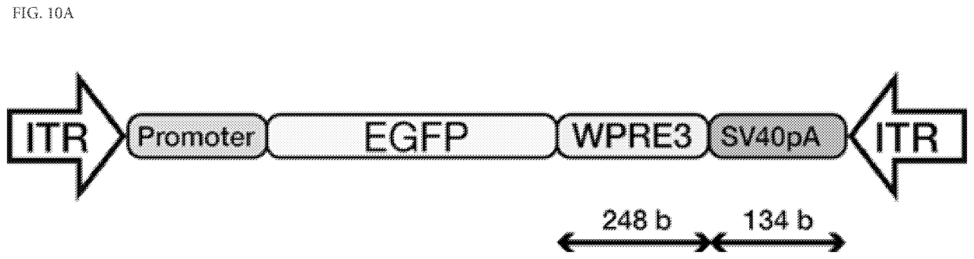

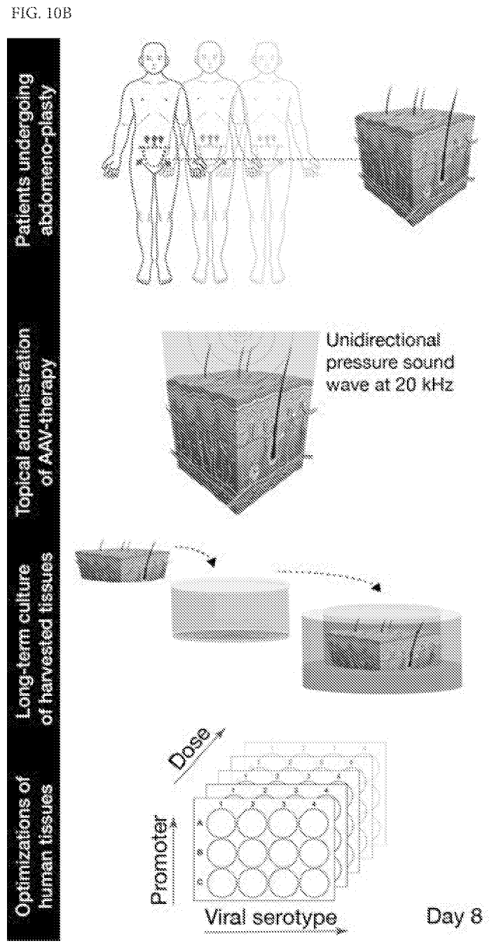

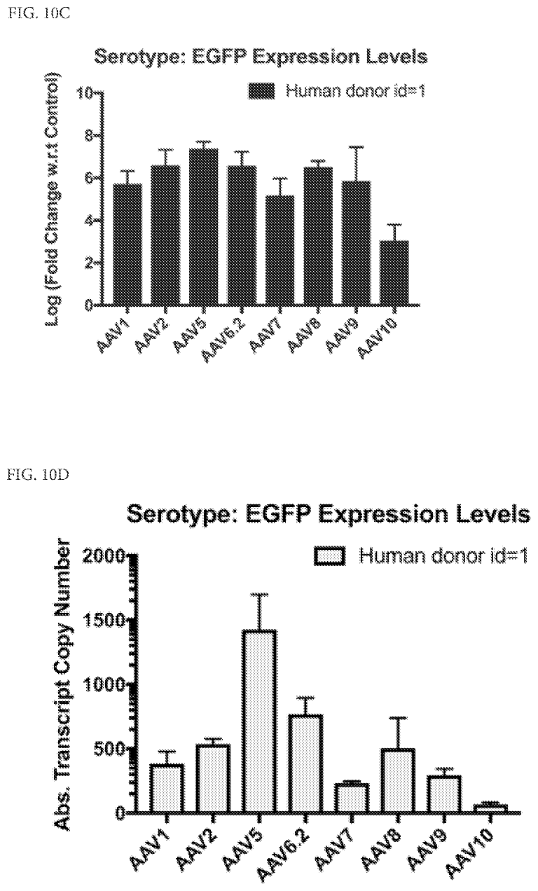

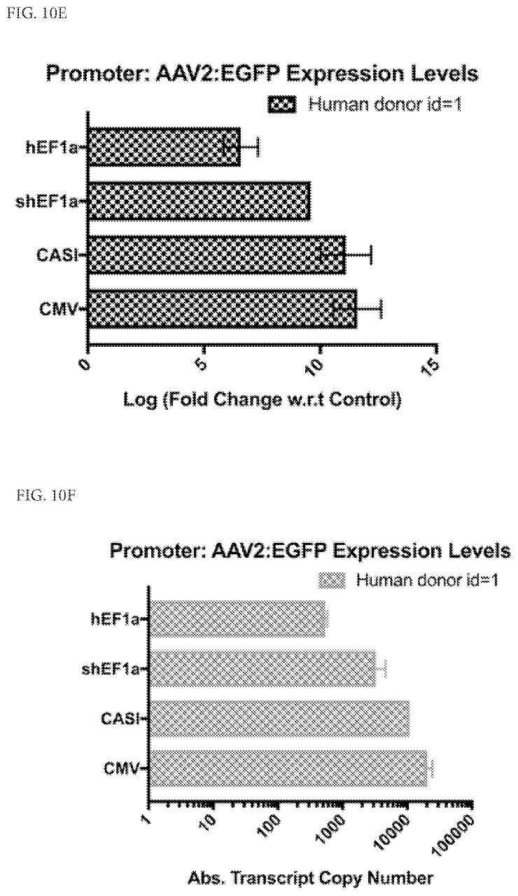

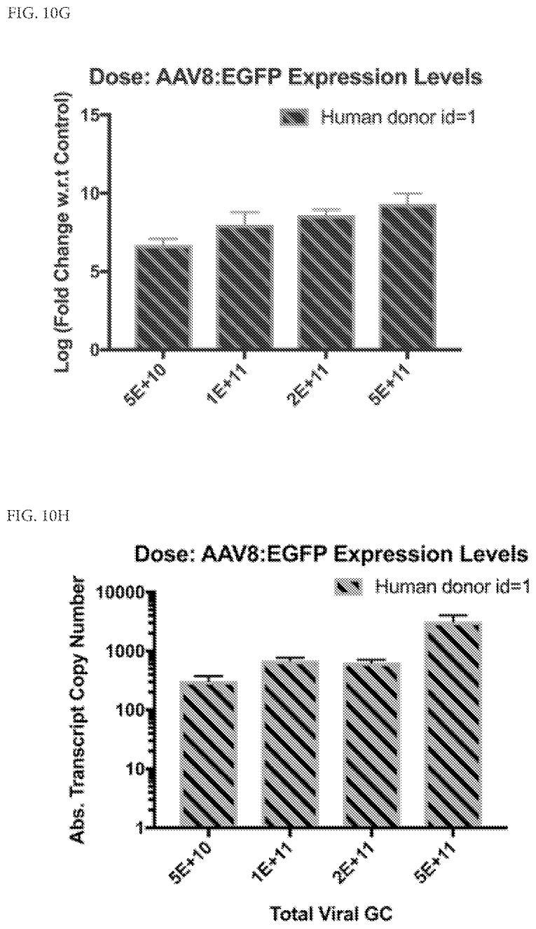

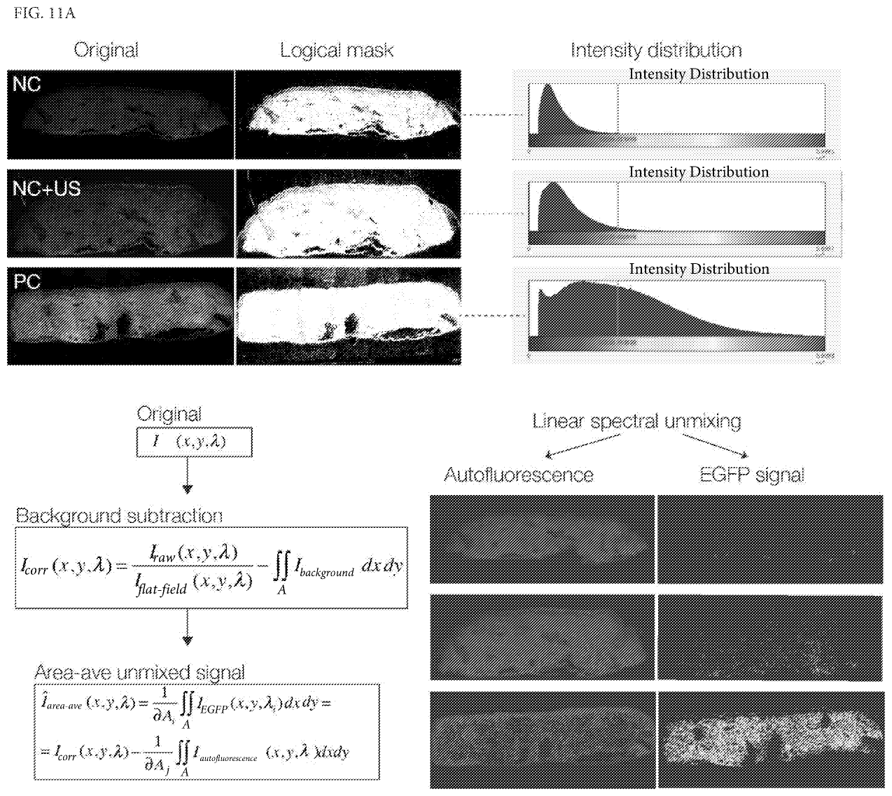

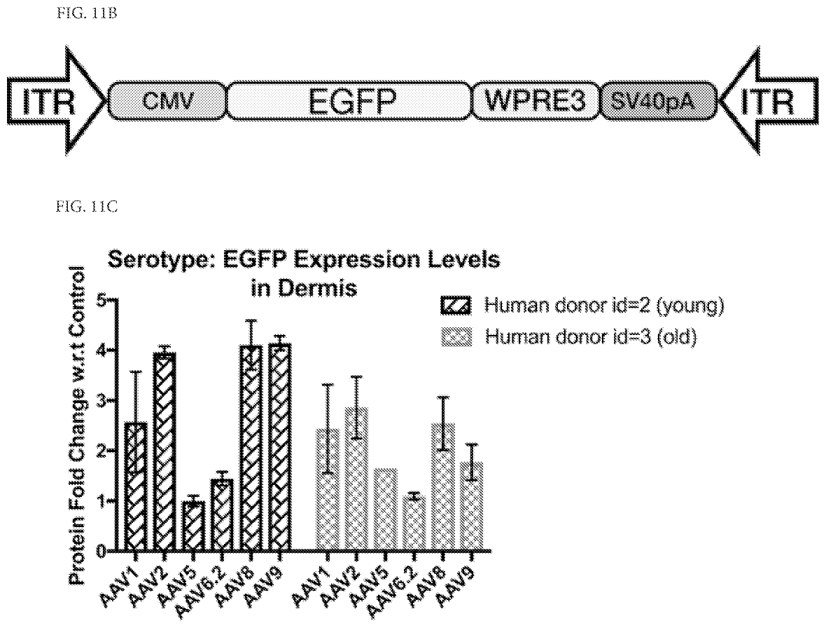

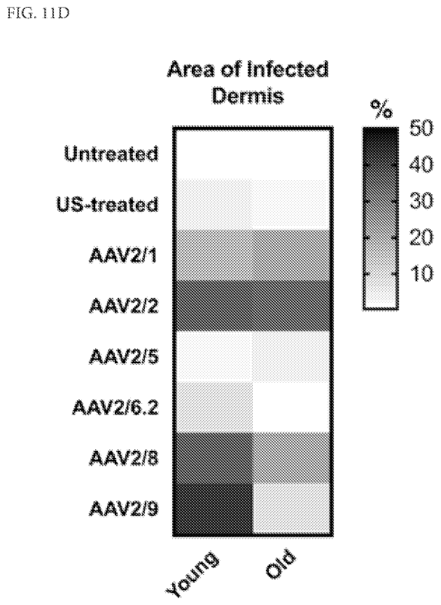

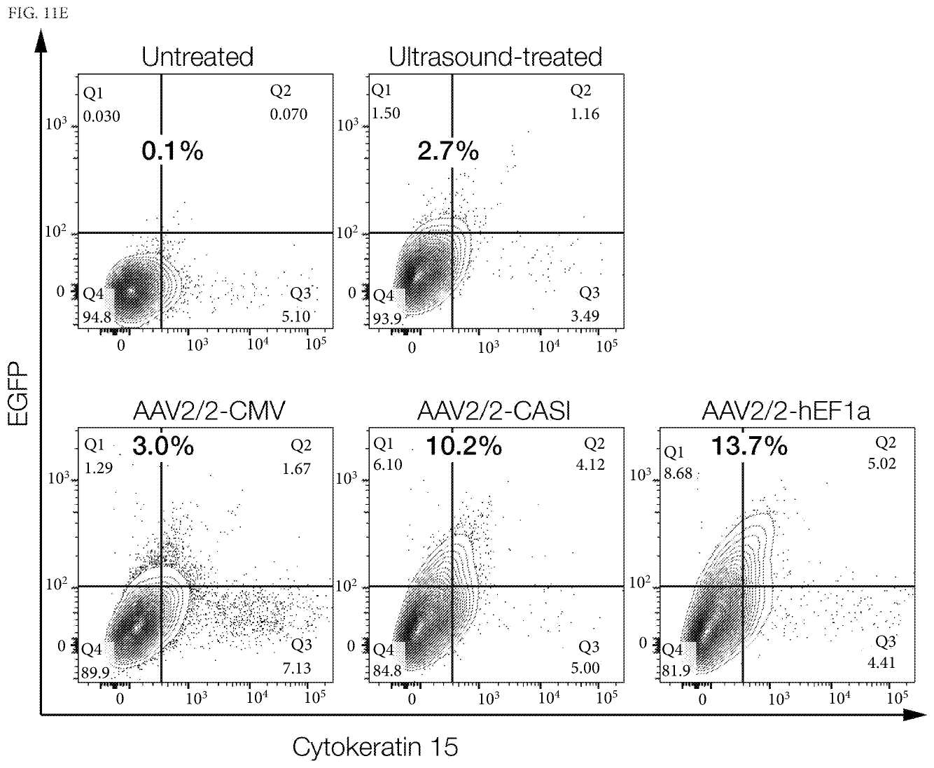

One of the hallmarks of skin stem cell aging is found that and deterioration in differentiation capacity is characterized with altered Carbon metabolism and TCA cycle, and aim to validate the functional properties of a herein reported regulator gene—Maleate dehydrogenase 2 (MDH2). MDH2 is a metabolic enzyme which is involved in processes associated with oxidation of malate to oxaloacetate by utilizing NAD/NADH cofactors in the Citrate cycle (TCA cycle), and affects energy consumption and metabolism between the mitochondria and cytosol. In FIGS. 9A-10B, protein production of MDH2 in aging skin progenitors of primary cultures were measured using Western blot, and it was confirmed that MDH2 levels gradually decrease with age, thus deeming it a valid biomarker for skin aging.

TABLE 3
Pathways id's: 1_Ribosome, 2_Oxidative phosphorylation,
3_Non-alcoholic fatty liver disease (NAFLD), 4_Protein processing in
endoplasmic reticulum, 5_Proteasome, 6_Metabolic pathways, 7_Protein export,
8_Carbon metabolism, 9_Citrate cycle (TCA cycle), 10_Glutathione
metabolism, and their associated gene members.

| | | Propagation | | | |
|---|---|---|---|---|---|
| ID | Pathway | score | Degree | Log2(Fold Change) | Label |
| ANPEP | 10 | 0.5518 | 44 | 1.5000 | ANPEP |
| ATF6B | 4 | 1.4319 | 66 | −0.5000 | ATF6B |
| ATP6V0C | 2 | 0.5240 | 174 | −0.7451 | ATP6V0C |
| CERS3 | 6 | 1.5163 | 14 | −0.5000 | CERS3 |
| COX4I1 | 2_3_6 | 1.2876 | 438 | −0.5000 | COX4I1 |
| COX5B | 2_3 | 0.9197 | 402 | −0.5000 | COX5B |
| COX6C | 2 | 0.5518 | 262 | −0.5000 | COX6C |
| DDIT3 | 3_4 | 0.9098 | 150 | −0.9325 | DDIT3 |
| DNAJC3 | 4 | 0.9098 | 78 | −0.9639 | DNAJC3 |
| G6PD | 8_10 | 0.3991 | 210 | −0.5000 | G6PD |
| GALNT5 | 6 | 1.0381 | 6 | −2.0850 | GALNT5 |
| GMDS | 6 | 2.7952 | 46 | 1.0850 | GMDS |
| GPT2 | 8 | 0.5518 | 24 | −0.5000 | GPT2 |
| GPX4 | 10 | 0.3033 | 426 | −1.9811 | GPX4 |
| GSTM1 | 10 | 0.3033 | 74 | −1.5473 | GSTM1 |
| GSTM3 | 10 | 0.3033 | 42 | −0.5000 | GSTM3 |
| GSTM4 | 10 | 0.5758 | 32 | −0.5000 | GSTM4 |
| GSTO1 | 10 | 0.3366 | 358 | −0.1374 | GSTO1 |
| GSTO2 | 10 | 0.3033 | 88 | −0.7035 | GSTO2 |
| HSPA5 | 7 | 0.5240 | 322 | 0.2655 | HSPA5 |
| IDH2 | 9 | 0.3008 | 236 | −0.5000 | IDH2 |
| IDH3B | 9 | 0.3033 | 324 | 1.0850 | IDH3B |
| IDH3G | 8_9 | 0.3991 | 244 | −1.9507 | IDH3G |
| INSR | 3 | 0.9098 | 260 | −1.2244 | INSR |
| MDH1 | 8_9 | 0.5986 | 412 | 0.8219 | MDH1 |
| MDH2 | 8_9 | 0.5692 | 570 | −0.9150 | MDH2 |
| MGST1 | 10 | 0.3033 | 54 | −0.5000 | MGST1 |
| MOGS | 4_6 | 1.2167 | 106 | 1.0850 | MOGS |
| NDUFA12 | 2_3_6 | 1.2876 | 500 | −0.7630 | NDUFA12 |
| NDUFS4 | 2_3 | 0.7288 | 342 | 0.5000 | NDUFS4 |
| NDUFS5 | 2_3 | 0.9197 | 576 | −0.5000 | NDUFS5 |
| NME2 | 6 | 1.0887 | 414 | −0.5000 | NME2 |
| NOS3 | 6 | 1.3995 | 240 | −0.5000 | NOS3 |
| OXA1L | 7 | 0.5240 | 208 | −0.5000 | OXA1L |
| PCK2 | 9 | 0.5518 | 68 | 3.0850 | PCK2 |
| PDIA3 | 4 | 1.0887 | 204 | −1.5995 | PDIA3 |
| PHGDH | 8 | 0.5240 | 142 | −0.5000 | PHGDH |
| POMP | 5 | 0.3746 | 474 | −0.8479 | POMP |
| PPA2 | 2 | 0.6260 | 204 | −1.0850 | PPA2 |
| PRDX6 | 6 | 1.2876 | 134 | −0.5000 | PRDX6 |
| PREB | 4 | 0.9098 | 172 | −0.5000 | PREB |
| PSMA5 | 5 | 0.3991 | 494 | −0.5000 | PSMA5 |
| PSMB4 | 5 | 2.0684 | 794 | −2.5000 | PSMB4 |
| PSMB6 | 5 | 0.3991 | 770 | −2.8219 | PSMB6 |
| PSMC3 | 5 | 2.0684 | 636 | −0.5000 | PSMC3 |
| PSMD14 | 5 | 0.3609 | 606 | −0.5000 | PSMD14 |
| PSMD2 | 5 | 2.0684 | 382 | 0.6890 | PSMD2 |
| PSMD4 | 5 | 0.5240 | 312 | −0.5000 | PSMD4 |
| PSMD8 | 5 | 0.3746 | 692 | −2.5000 | PSMD8 |
| PSMF1 | 5 | 0.9098 | 94 | 0.5000 | PSMF1 |
| RBX1 | 4 | 1.2876 | 386 | −0.5000 | RBX1 |
| RPL11 | 1 | 1.2876 | 548 | −0.5000 | RPL11 |
| RPL24 | 1 | 2.0684 | 530 | −0.5000 | RPL24 |
| RPL3 | 1 | 2.0684 | 376 | −1.4888 | RPL3 |
| RPL31 | 1 | 2.0684 | 362 | −1.0850 | RPL31 |
| RPL37 | 1 | 2.0684 | 318 | −0.5000 | RPL37 |
| RPL39 | 1 | 2.7952 | 318 | −0.7224 | RPL39 |
| RPL7 | 1 | 2.0684 | 366 | −1.5000 | RPL7 |
| RPS11 | 1 | 2.0684 | 328 | −1.0146 | RPS11 |
| RPS24 | 1 | 1.2876 | 392 | 0.5000 | RPS24 |
| RPS4X | 1 | 2.0684 | 280 | −3.0850 | RPS4X |
| RRM1 | 10 | 0.3033 | 308 | −1.0369 | RRM1 |
| SDHB | 9 | 0.3033 | 392 | 0.9854 | SDHB |
| SDHC | 9 | 0.3366 | 228 | −0.5000 | SDHC |
| SDHD | 2_3_8_9 | 0.9098 | 214 | −0.5000 | SDHD |
| SDSL | 8 | 0.5955 | 30 | −1.0850 | SDSL |
| SEC11C | 7 | 0.3033 | 238 | −0.5000 | SEC11C |
| SEC61A1 | 7 | 0.3033 | 392 | −0.5000 | SEC61A1 |
| SEC61B | 7 | 0.3746 | 328 | −0.5000 | SEC61B |
| SEC61G | 7 | 0.3991 | 530 | −0.5000 | SEC61G |
| SEL1L | 4 | 0.9098 | 102 | −0.6354 | SEL1L |
| SHMT2 | 8 | 0.5240 | 346 | −0.5000 | SHMT2 |
| SPCS1 | 7 | 0.3033 | 552 | −0.3074 | SPCS1 |
| SPCS2 | 7 | 0.3024 | 284 | −0.8536 | SPCS2 |
| SPR | 6 | 1.8711 | 16 | 0.1781 | SPR |
| SRP14 | 7 | 0.3033 | 424 | −0.5000 | SRP14 |
| SRP19 | 7 | 0.3033 | 402 | −0.4198 | SRP19 |
| SUCLG1 | 9 | 0.3173 | 312 | −0.5000 | SUCLG1 |
| TALDO1 | 8 | 0.5240 | 346 | 0.3301 | TALDO1 |
| TNF | 3 | 0.9098 | 1368 | −1.0850 | TNF |
| UQCRH | 2 | 0.5943 | 576 | 1.0850 | UQCRH |
| WFS1 | 4 | 0.9098 | 48 | −5.0850 | WFS1 |
| XBP1 | 3_4 | 1.0381 | 140 | −0.5000 | XBP1 |

According to certain embodiments, genes relating to skin aging as disclosed herein represent skin aging biomarkers and their expression can be modulated by the methods disclosed herein to promote skin function and health. According to certain embodiments, the disclosed method comprises delivery of genes comprising sequences of SEQ ID NOS 1-122 to the skin or delivery of genes that modulate the expression of the genes comprising sequences of SEQ ID NOS 1-122.

Lengthy table referenced here
US20200375868A1-20201203-T00001
Please refer to the end of the specification for access instructions.

### Example IV

### Optimization of Gene Transfer to Whole Skin

To create an optimal framework for delivery of transgenes to skin cells, fluorescent enhanced GFP reporter transgene was cloned in an AAV vector containing AAV2-derived inverted terminal repeat 1 (ITR1) in the flip direction and an inverted terminal repeat 2 (ITR2) in the flop direction. The ITR1 element is annealed to human hEF1a (human elongation factor-1 alpha) promoter, while ITR2 element is annealed to 134b-long SV40 late polyadenylation (truncated SV40 late poly(A)) element and 248b-long WPRE3 (truncated) element. FIG. 10A shows a schematic of a length-optimized modular vector with EGFP gene inserted. The gene is flanked by unique SpeI and NotI restriction sites. The gene is preceded by a Kozak sequence (GCCACC) and is terminated by a (TAA) stop codon, prior to the NotI restriction site.

To evaluate the efficacy of gene transfer to human skin cells, a variety of AAV capsids were used to make hybrid AAV viral serotypes. A typical workflow is shown on FIG. 10B. These vectors were delivered topically to human skin explants pre-treated with low frequency (20 kHz) ultrasound. Sonic wave with a period 30 sec and duration of 3 cycles was generated to permeabilize abdominal human skin by disrupting its cornified layer—the stratum corneum. Recombinant AAV viruses of serotypes 2/1, 2/2, 2/5, 2/6.2, 2/7, 2/8, 2/9, and 2/10 were administered at a dose of 2E+11 GC per 1.2 cm-dia full-thickness human skin. After AAV-treatment, human skin explants were cultured in 1 cm-transwells for 8 days after which tissues were analyzed for gene expression. As shown on FIG. 10C, the capsids of AAV2/5, AAV2/2, AAV2/6.2, and AAV2/8 gave the most robust gene expression characterized by gene expression of the transgene in whole skin lysate. Reported gene expression values were normalized to endogenous Active-beta (ACTB) levels relative to a control untreated tissue. Next, in FIG. 10D, the absolute gene expression copy number was evaluated based on a standard curve built upon known amounts of input transgene. Similarly, AAV2/5, AAV2/6.2, AAV2/2, and AAV2/8 presented with the highest expression values. FIGS. 10C and 10D show mean and standard error to the mean of N=2 replicates.

To determine the expression potential of a panel of viral promoters in human skin tissue, a group of ubiquitous and tissue-specific promoters was tested in human skin explants as represented in the workflow of FIG. 10B. The tested panel included cytomegalovirus immediate early promoter (CMV), CASI promoter (a fusion of cytomegalovirus immediate early promoter (CMV) followed by a fragment of chicken-f-actin (CAG) promoter), short human elongation factor-1 alpha (shEF1a), and human elongation factor-1 alpha (hEF1a). Recombinant AAV2 serotype at a dose of 2E+11 GC per 1.2 cm-dia full-thickness human skin was administered to all tissue explants. The strength of human skin cell expression of each promoter was evaluated by the gene expression of reporter gene, EGFP both in terms of relative (to negative control) expression (FIG. 10E) and absolute copy number expression (FIG. 10F). FIGS. 10E and 10F show mean and standard error to the mean of N=2 replicates. Within the duration of the experiment at day 8, CMV and CASI presented with the highest expression potential while shEF1a presented with levels on the same order.

To confirm dose-dependency within the range of use, AAV2/8-hEF1a-EGFP was administered to human skin explants at the doses of 5E+10, 1E+11, 2E+11, and 5E+11 GC. The strength of cell expression was evaluated by the gene expression of reporter gene, EGFP both in terms of relative (to negative control) expression (FIG. 10G) and absolute copy number expression (FIG. 10H). A typical dose of 2E+11 yielded a total of 642 EGFP transcripts.

### Example V

### Optimization of Gene Transfer to Human Skin Dennis

To establish delivery efficiency selectively to dermal skin cells, the native fluorescence of a reporter gene, EGFP was measured over a large surface area in full thickness human breast skin tissues (16 mm×2 mm in cross-sectional area) maintained in culture conditions post-treatment. Human skin explants were harvested 24 hours after the treatment, and embedded in OCT. To determine the total signal over the cross-sectional area of the dermis (16 mm×1 mm×20 μm), native GFP fluorescence was quantified using a custom image post-processing pipeline in MatLab. The algorithm executes flat-field and background corrections, creates a logical mask of the image, and performs linear un-mixing of the total fluorescence intensity based upon different emission spectra of tissue auto-fluorescence and signal due to the expression of EGFP. The process is shown in FIG. 11A for one untreated, one ultrasound-treated, and one AAV-treated tissue sample. A schematic illustration of AAV-CMV-EGFP vector is shown in FIG. 11B Recombinant AAV viruses of serotypes 2/1, 2/2, 2/5, 2/6.2, 2/8, 2/9 were administered at a dose of 2E+11 GC per tissue explant and the fluorescence signal is reported for two donors, one young (of ages 30) and one old (of age 52) in FIG. 11C. While expression levels differed between the two human donors, the optimal gene expression in dermal cells was consistent and the highest for AAV2/8, AAV2/2, AAV2/9, and AAV2/1. The highest amount of protein expression reached 4-fold over that of an untreated-tissue, and covered nearly 50% of the cross-sectional dermal area in the young donor, as shown on the heatmap of FIG. 11D. Large variation was observed between the two donors.

The infectivity of the CMV promoter in human dermal cells shows a transient response over time and is the highest during the first week of infection. To quantify longer term expression response in the human dermis, skin explants (from human donor id=4 of medium age) were AAV2-infected with CMV, CASI, and hEF1a promoters and harvested at Day 12. To separate epidermis from dermis, explants were treated with a protease (dispase II at 5U/ml, overnight) to facilitate peeling off the epidermis. A population of dermal cells (predominantly skin fibroblasts) was then dissociated using Collagenase I at 1 mg/ml in DMEM/Serum (20%) solution at 37 C. The isolated cells were stained with anti-EGFP and anti-Cytokeratin 15 antibodies for processing with FACS. A population of ˜30,000 cells was analyzed. As shown on FIG. 11E, the populations of single EGFP-positive cells and double EGFP/K15-positive cells were summed and the highest infectivity capacity yielded a total of 13.7% for the hEF1a-driven AAV vector.

### Example VI

### Optimization of Gene Transfer to Human Skin Epidermis

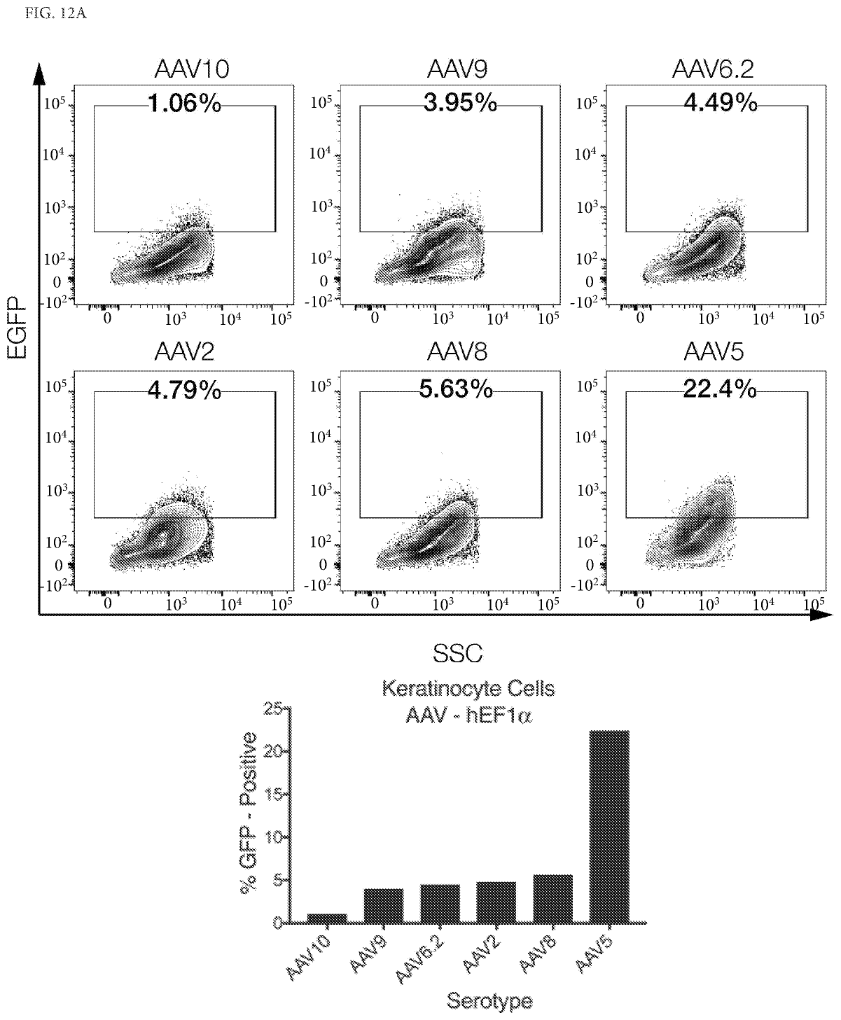

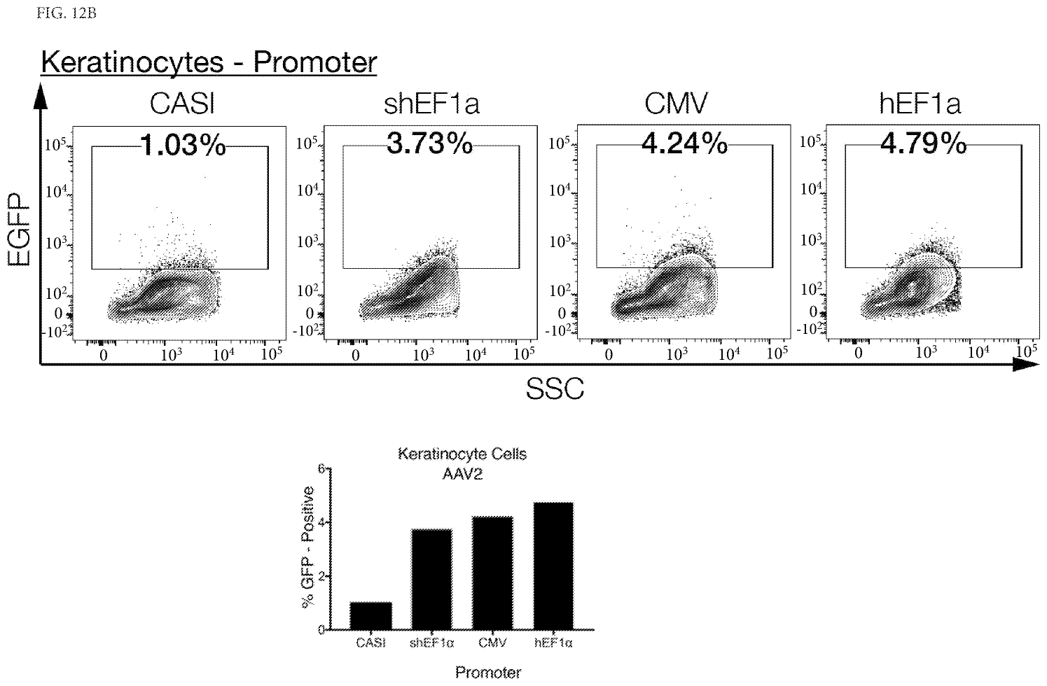

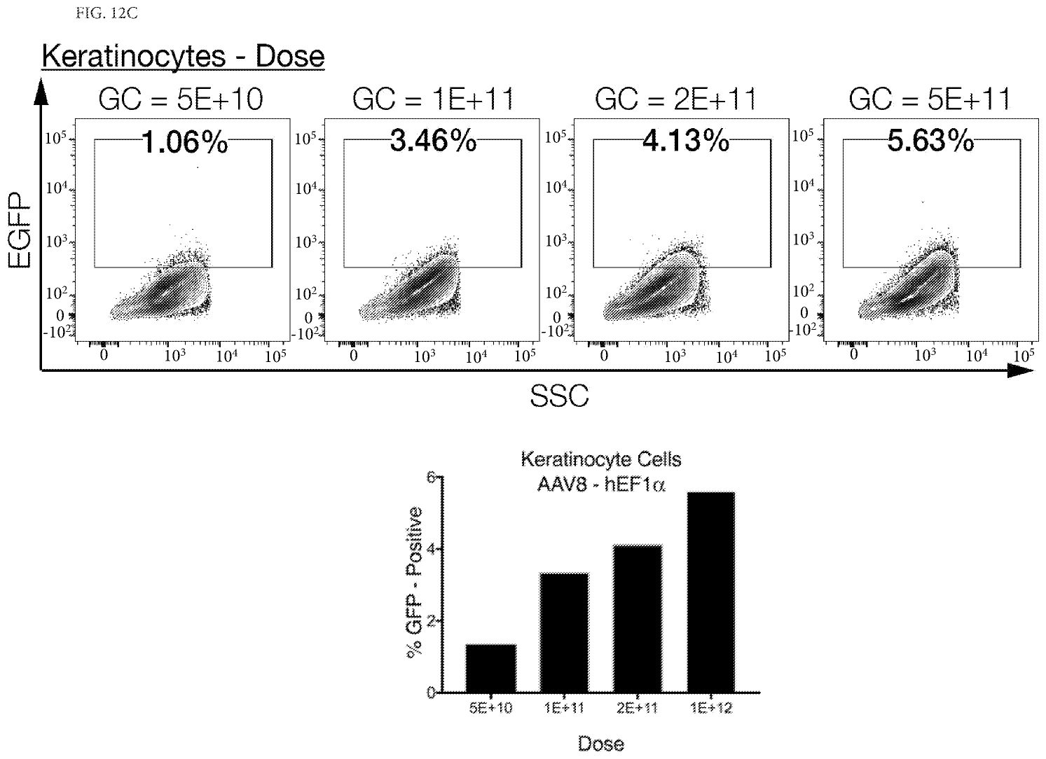

The expression potential of recombinant AAV virus to infect and deliver genes to human epidermis was quantified by flow cytometry. Whole skin was permeabilized using topical ultrasonic treatment, after which it was spot-treated with therapy. In one instance, skin explants (from human donor id=4 of medium age) were AAV-treated with hybrid serotypes of AAV2/2, AAV2/5, AAV2/6.2, AAV2/8, AAV2/9, and AAV2/10 at a dose of 2E+11 GC per explant, and cultured for 12 days. All vectors were driven by the hEF1a promoter. The epidermis of the explants was separated from the dermis using an overnight protease treatment (dispase II at 5U/ml), and keratinocyte cells were dissociated with Trypsin-EDTA (0.25%) for 15 min at 37 C. The dissociated cells were stained with an anti-EGFP antibody and quantified for expression of GFP. As shown in FIG. 12A, AAV2/5 provided the highest GFP signal and the most robust expression of 22.4% in total epidermal keratinocyte cells. In another instance, the efficacy potential of CMV, CASI, shEF1a, and hEF1a promoters was evaluated using AAV2/2 at a dose of 2E+11 GC per explant. hEF1a, CMV, shEF1a (truncated version) presented comparable efficiencies (FIG. 12B). Dose dependency response was evaluated using AA8-hEF1a from 5E+10 to 5E+11 GC per explant. As seen on FIG. 12C, dose response did not yield a linear response in the epidermis.

### Example VII

### Optimization of Gene Transfer to Human Skin Stem and Progenitor Cells

Skin is maintained through a balance of proliferation, differentiation, and self-renewal of stem cells that take place during normal tissue homeostasis or tissue repair. The epidermis relies on a population of stem cells and proliferating progenitors to continuously maintain its barrier-protective function. While the epidermal differentiated population (mature keratinocytes) has a lifespan of ˜4 weeks, the stem and progenitor cell populations have a nearly life-long span.

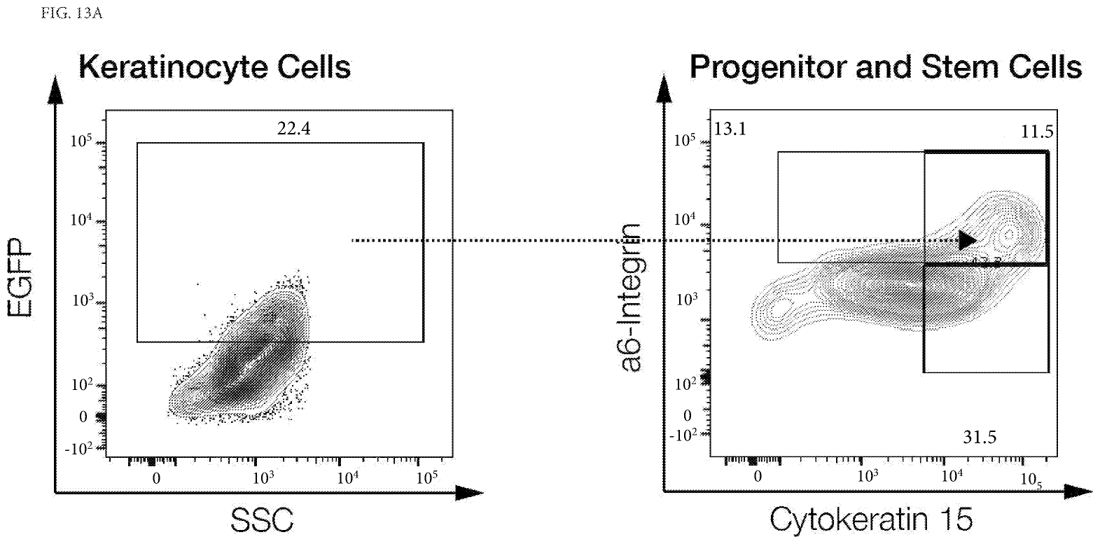

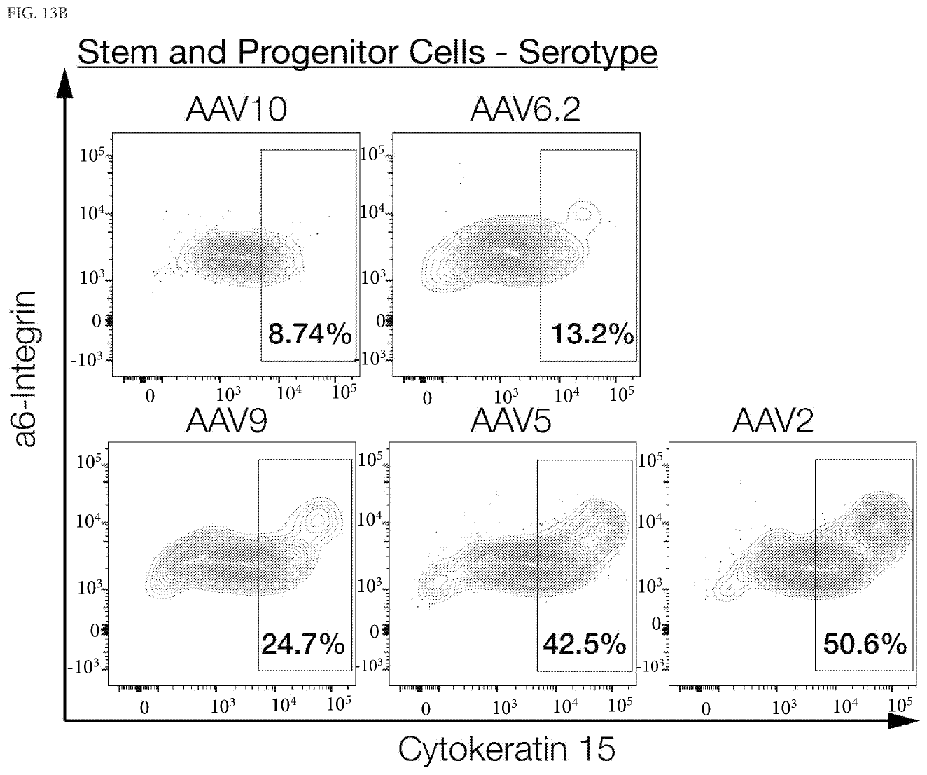

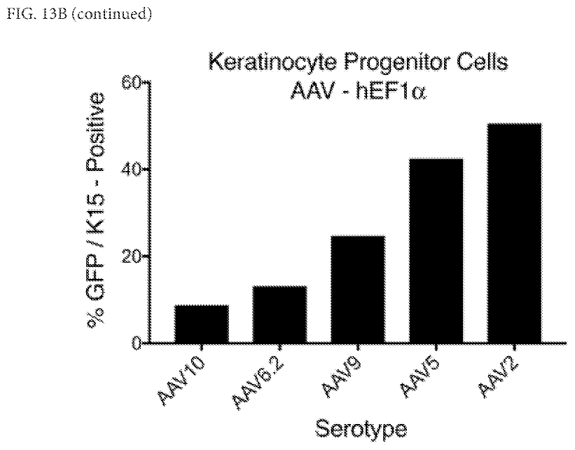

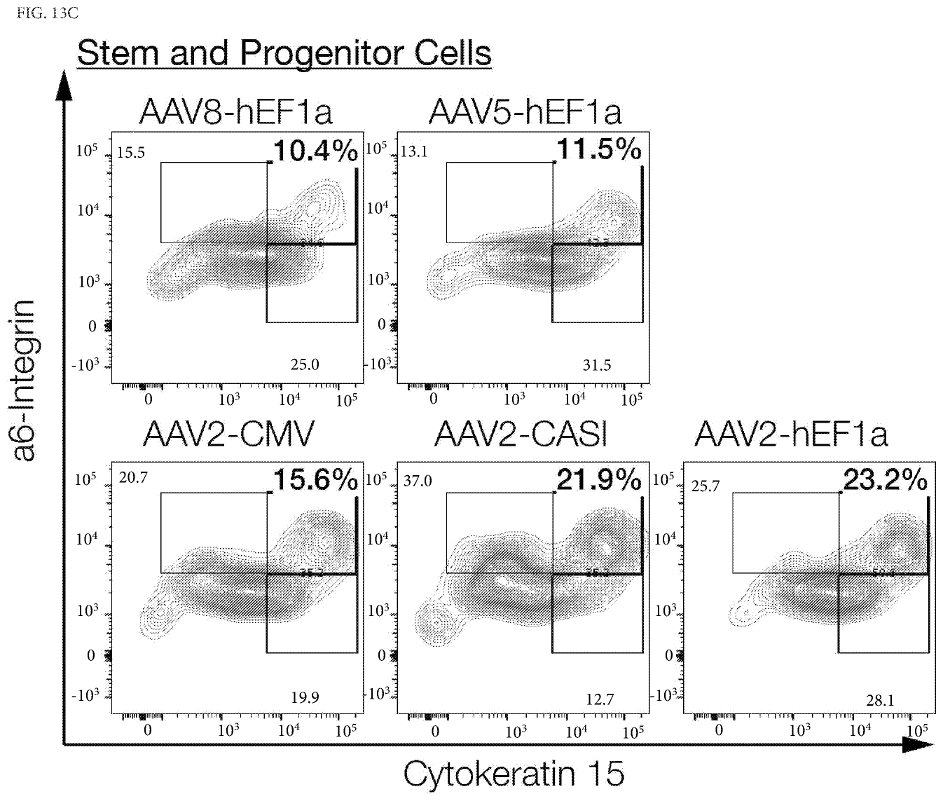

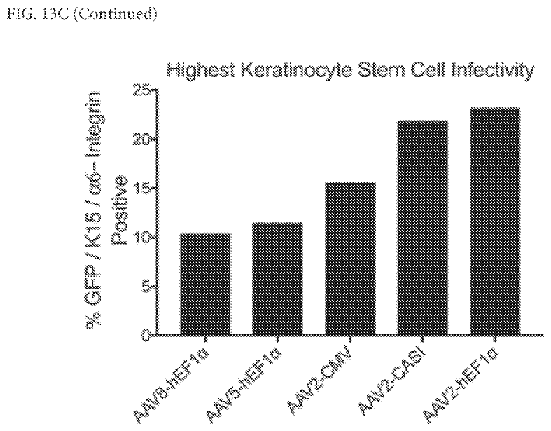

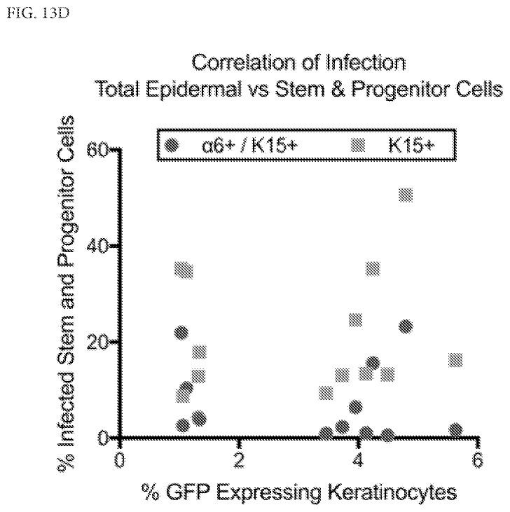

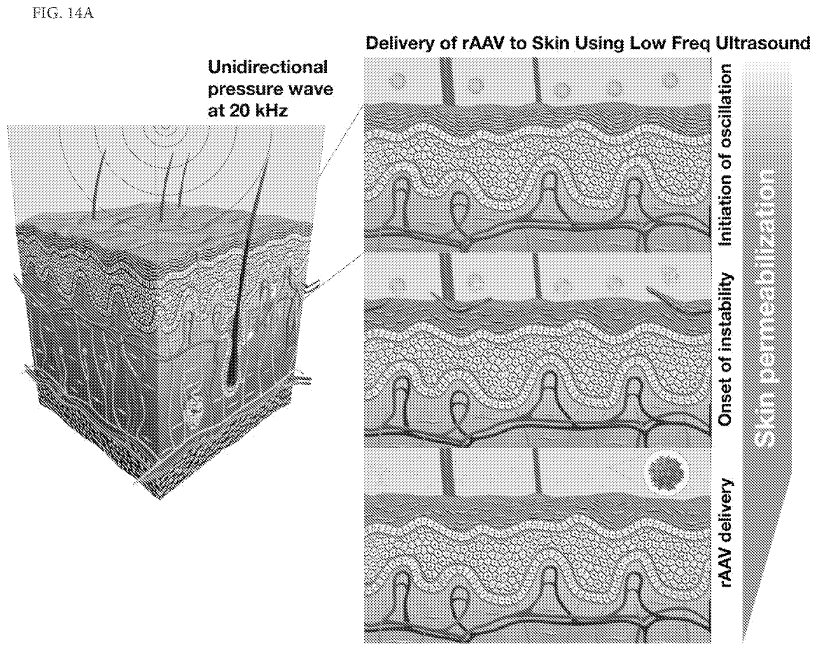

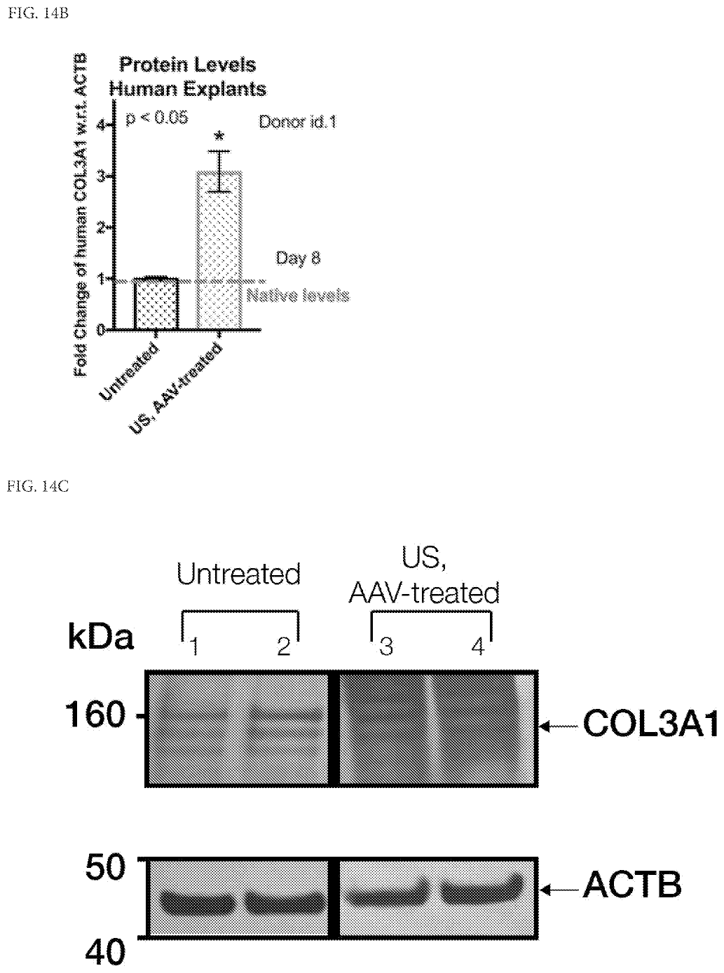

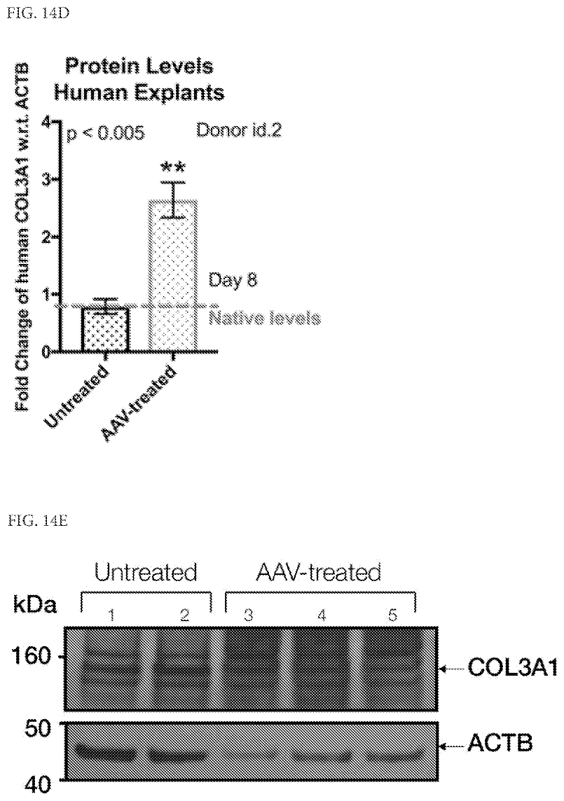

To achieve long-term expression of genes in skin tissues, gene transfer to the populations of stem cells located within the basal membrane (slow cycling stem cells and their transiently amplifying progenitors) was optimized. As shown in FIG. 14A, the differentiated keratinocyte population was further analyzed for therapy efficacy towards progenitor stem cells expressing markers either for Cytokeratin 15, a6-Integrin, or both. Based on their ability to infect progenitor and stems cells, the top 5 most efficacious AAV-serotypes measured by GFP and K15 signal are listed in FIG. 13B. In the K15+progenitor and stem cell populations, AAV2/2 and AAV2/5 presented with 50.6% and 42.5% infectivity efficiency, respectively.

Across all examined viral vectors, the ones with the highest infectivity capacity in the epidermal progenitor and stem cell populations expressing K15 and a6-integrin were AAV2/2-hEF1a, AAV2/2-CASI, AAV2/2-CMV, AAV2/5-hEF1a, and AAV2/8-hEF1a. As shown in FIG. 13C, the best performing vector AAV2/2-hEF1a which stained for 50.6% K15, stained for 23.2% of K15 and a6-integrin, while AAV2/5-hEF1a (42.5% K15) showed 11.5% signal for K15 and a6-integrin. Both AAV2/2 and AAV2/5 serotypes, driven by hEF1a promoter presented with high infectivity towards epidermal stem and progenitor cells, but AAV2/5 presented with higher infectivity towards differentiated keratinocytes. The correspondence between % GFP-positive epidermal cells and % GFP-positive stem and progenitor cells was mapped in FIG. 13D, and no correlation was determined between total capacity of infection, and stem cell capacity of infectivity.

### Example VIII

### Ex Vivo Human Expression of Collagen Transgenes

Recombinant AAV2/2 virus that expresses human collagen III (alpha domain) driven by a truncated hEF1a promoter was administered to human skin explants at a dose of 2E+11 GC per sample. Ultrasound-mediated gene delivery was executed in a single step, and the process of skin permeabilization required ultrasonic initiation of vibrating cavitational bubbles, active oscillation, instability and bursting of bubbles followed by topical, passive-diffusion delivery of a single therapy dose (FIG. 14A). Type III collagen is a human gene encoding collagen III fibrils, which serve as a major component of the skin extracellular matrix and is primarily produced by dermal fibroblast cells. Within 8 days of administration, Collagen III levels started to increase over the native amounts present in the skin explants, and reached significant amounts (p<0.05) of as high as 3.5-fold overproduction compared to the negative, untreated control (FIG. 14B). FIG. 14B shows the mean and standard error for N=2 human explants. Protein levels for Collagen III were analyzed by Western blot (FIG. 14C).

To determine the robustness of dermal matrix remodeling and age-associated thinning of the dermis by modulation of collagen III, three human explant from another donor were treated with the same rAAV virus encoding collagen III protein. rAAV2/2 virus was administered to human skin which was cultured for 8 days and analyzed for levels of collagen III using Western blot. The highest amount of collagen III expression reached 3.2-fold overproduction compared to the native levels in the control tissue. FIG. 14D shows significant levels of overexpression (p<0.005) and presents the mean and standard error for N=3 human explants. Western blot images are shown in FIG. 14E.

This example shows that the recombinant AAV virus expressing collagen III can be effectively used to provide consistent protein overexpression with the human dermis.

### Example IX

### In Vivo Skin Rejuvenation by Modulation of 4 Age-Related Genes

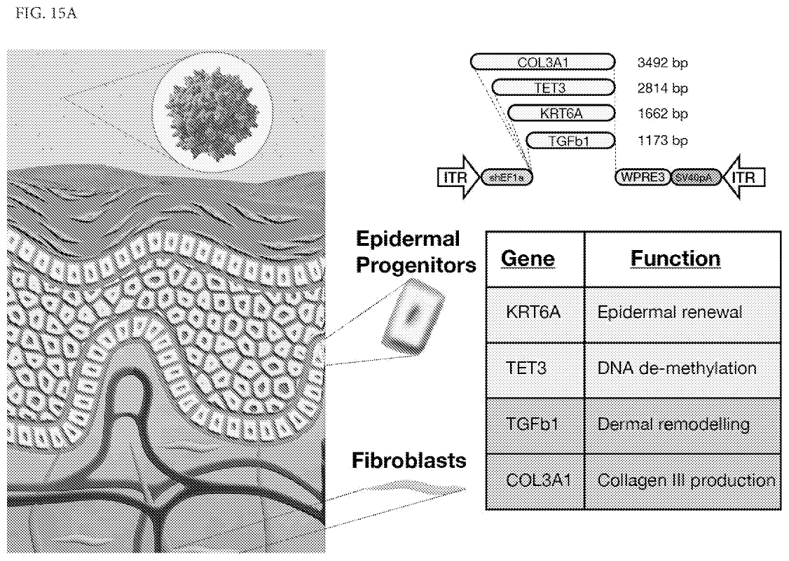

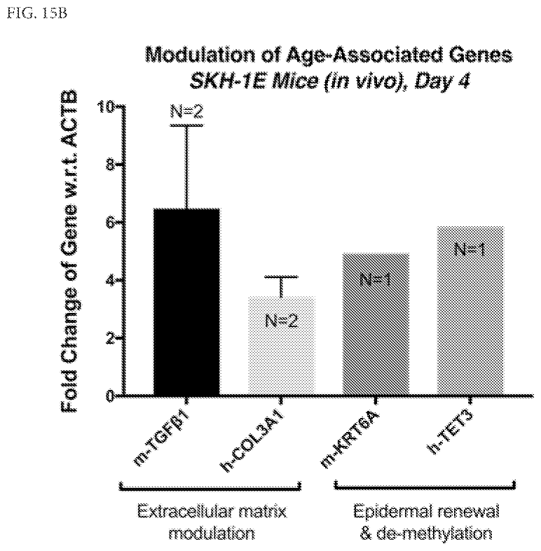

This example describes modulation of 4 age-related genes—(mouse) KRT6A, (human) TET3, (mouse) TGFb1, and (human) COL3A1 in SKH-1E hairless mice. KRT6 Å and TET3 target the epidermis and improve functions related to stem and progenitor cell renewal and DNA de-methylation, while TGFb1 and COL3A1 target the dermis and modulate production of extracellular proteins (as shown in FIG. 15A). Recombinant AAV virus was produced—recombinant AAV of serotype 2 was used to express COL3A1, TET3, and KRT6A, while recombinant AAV of serotype 8 was used to express TGFb1. The rAAV vector consisted of swappable transgene flanked by unique NheI and NotI restriction sites. The rAAV vector is driven by a length-optimized promoter shEF1a (truncated hEF1a), WPRE3 and SV40 pA of sizes 231b, 248b, and 134b, respectively. These length optimizations permitted packaging and expression of larger transgene as in the case of COL3A1. All vectors were administered at one location via a single US-permeabilization treatment at a dose of 2E+11 GC per animal. At Day 4, skin tissues were harvested and whole tissue lysates were analyzed by RT-qPCR to measure expression levels of the transferred transgenes. As shown on FIG. 15B, all modulated transgenes increase in expression ranging from 3.4-fold to 6.5-fold relative to a negative (untreated) control tissue.

### Example X

### In Vivo Skin Rebuilding of Skin's Extracellular Matrix by Long-Term Expression of Collagen III

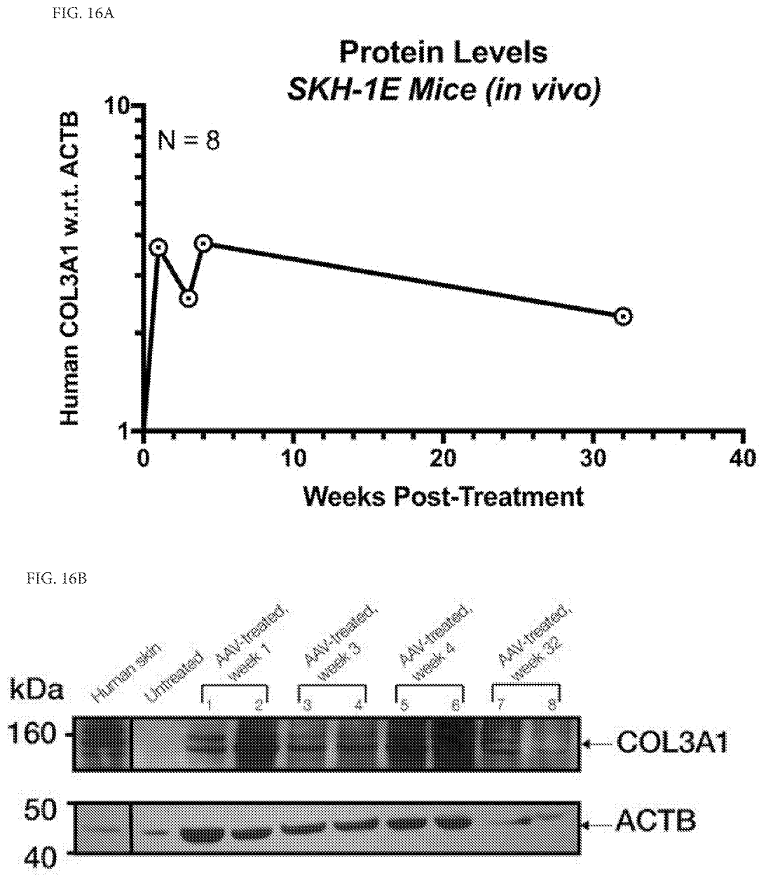

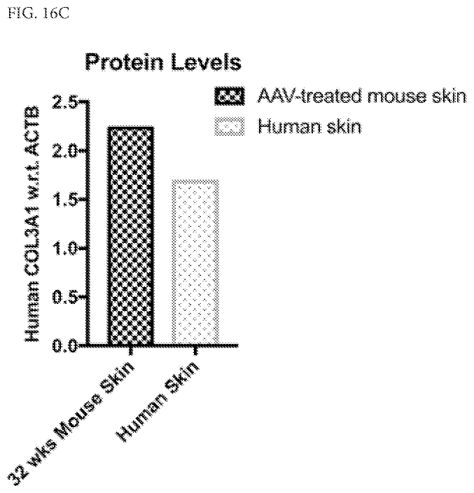

To determine expression capabilities of rAAV in hairless mice as a function of time, rAAV vector expressing collagen III was administered at a dose of 2E+11 GC per animal. FIG. 16A shows a protein expression curve as a function of time from 1 week to 32 weeks. Protein expression started to rise reaching 4-fold overexpression one week after administration and up to 3 weeks, after which it decreases one-fold to 2.25-fold. FIG. 16A represents a time curve of collagen III production which was maintained for at least 32 weeks after which the experiment was stopped. Expression protein levels were determined by Western blot on mouse skin lysates for N=8 mice (FIG. 16B). In parallel, collagen III levels were analyzed in human skin and levels were compared relative to the last data point in the mouse in vivo experiment (FIG. 16C). The native amounts of collagen III in human skin were lower than the newly produced amount in mouse skin after 32 weeks-1.7-vs 2.25-fold, respectively.

This example shows that the recombinant AAV virus disclosed here can be used to drive effective and robust expression of proteins over long periods of time.

### Example XI

### Ultraclean Production and Purification of Recombinant AAV

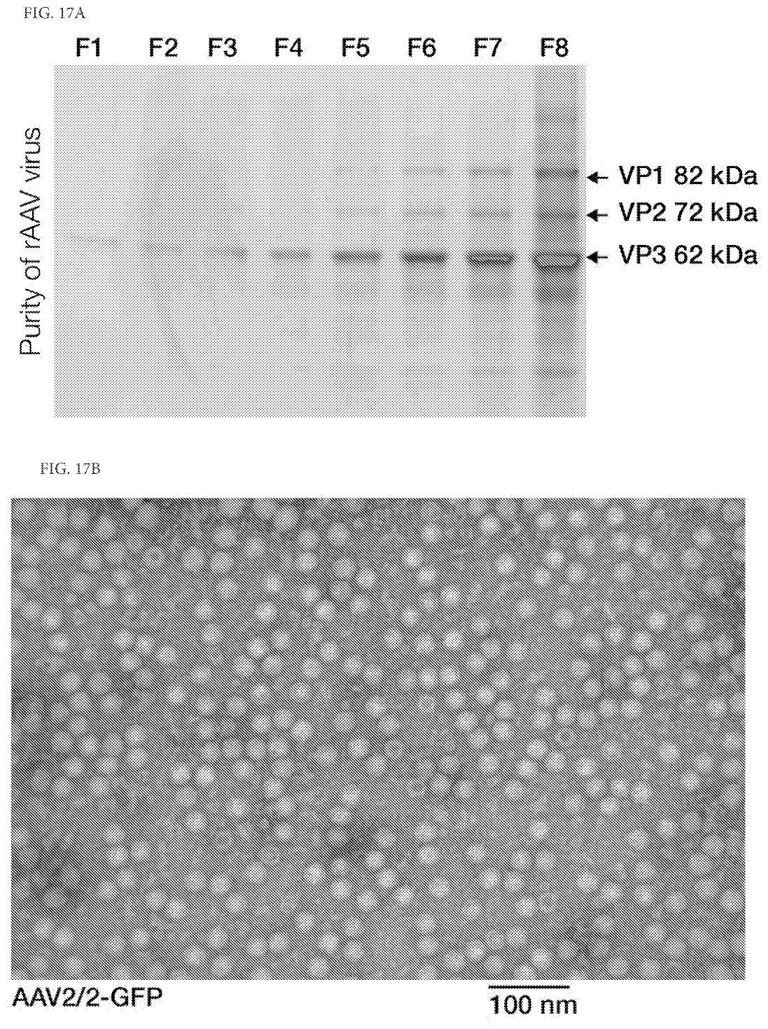

HEK293T Cells (ATCC) were expanded in DMEM (Corning) with 10% FBS (Genessee) and 1% Pen/Strep (Life) and cultured on 5-Layer Flasks (Corning) until 70-80% confluent. On Day 0, helper plasmid, capsid plasmid, and transgene ITR plasmid were combined with PEI Max (Polysciences) in a triple plasmid transfection. On Day 3, additional complete media was added to the culture (50% of original volume). On Day 6, NaCl was added to each flask to a final concentration of 0.5M and incubated for 2 hours. Lysed and dissociated cells were then collected and stored overnight at 4 C. On Day 7, the supernatant of the cell lysate was collected and 0.22 μM sterile filtered before addition of PEG 8000 (Calbiochem) to a final concentration of 8% and left at 4 C overnight on a stirplate. On Day 8, PEG mixture was centrifuged at 4000G for 20 minutes. Supernatant was discarded, and pellet resuspended with PBS to a final volume of 8 mL. Benzonase (Millipore) was added and incubated at 37 C for 45 minutes. An ultracentrifuge gradient in optiseal tubes (Beckman-Coulter) was then created by layering resuspended PEG pellet, then 15%, 25%, 40%, and 60% iodixanol (Sigma) from the bottom-up. Samples were then balanced before ultracentrifugation at 240,000G for 1 hour. Tubes were then punctured on the bottom before collecting 500 μL fractions, stopping at the 40-25% interface. Samples were run on a protein gel and fractions with high VP protein purity (FIG. 17A), after which pooled and concentrated using Amicon 100 kDa spin filters (Millipore). PBS with 5% sorbitol and 0.001% Pluronic F68 (Gibco) was added to each tube before an additional spin to wash virus. Concentrated and washed virus was then titered via probe-based qPCR against the WPRE3 region on the capsid. The quality of the virus was visually inspected using transmission electron microscopy, and it was determined that more than 95% of the viral capsids were fully packaged, as shown on FIG. 17B for AAV2/2 expressing EGFP.

### Example XII

Ex Vivo Human Immune Response to rAAV

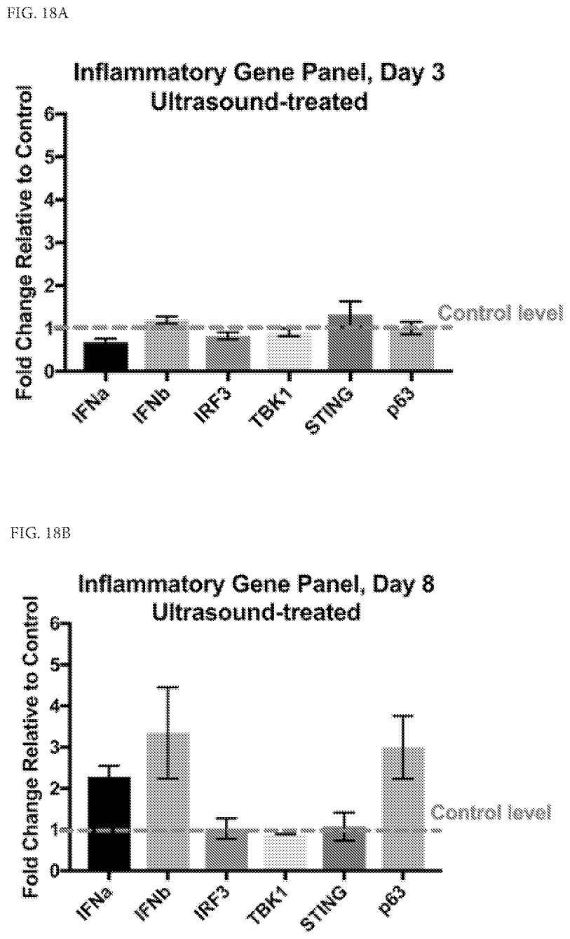

This example illustrates the therapeutic use of genetically engineered skin patch for in vivo immunomodulation, immunoprophylaxis, and passive delivery of therapies for diseases such as cancers, autoimmune diseases, metabolic disorders and viral infections. The virtual therapeutic skin patch is primed with transgenes serving preventative or therapeutic use by non-invasive delivery of recombinant AAV virus whose penetration to the epidermal and dermal layers is enabled by topical cavitational, low-frequency ultrasound method. Ultrasonic disruption of the stratum corneum is reversible and facilitates rAAV transport into the epidermis, the papillary and reticulous dermis avoiding injury and inflammation of the treated and surrounding tissues. FIGS. 18A-18B show an inflammatory panel at Day 3 and Day 8, respectively, run on epidermal cells dissociated from human skin explants after treatment with rAAV-GFP therapy via ultrasound. At Day 3 (FIG. 18A), no inflammatory response above the baseline levels was detected, while at Day 8 (FIG. 18B) a minor transient response was observed as evidenced by slightly increased gene expression levels of Interferon alpha-1 (INFa1) and Interferon beta-1 (INFb1). However, no acute innate response to the virus was detected as visible from the stable levels of Interferon regulatory factor 3 (IRF3), Serine/threonine-protein kinase (TBK1), and Stimulator of interferon genes protein (STINK). Moderately elevated levels of Tumor protein P63 (p63) is indicative of normal cell proliferation activity in the keratinocyte population.

The contents of all references, patents and published patent applications cited throughout this application are hereby incorporated by reference in their entirety for all purposes.

### OTHER EMBODIMENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

LENGTHY TABLES
The patent application contains a lengthy table section. A copy of the table is available in electronic form from the USPTO web site (https://seqdata.uspto.gov/?pageRequest=docDetail&DocID=US20200375868A1). An electronic copy of the table will also be available from the USPTO upon request and payment of the fee set forth in 37 CFR 1.19(b)(3).

1. A method of delivering a recombinant virus to a skin tissue comprising
applying ultrasound to the skin tissue, and
administering the recombinant virus to the skin tissue.

2. The method of claim 1 wherein the recombinant virus is delivered to the skin tissue of a subject in vivo.

3. The method of claim 2 wherein the skin tissue comprises native and autogeneic, isogeneic, xenogeneic and allogeneic skin tissue.

4. The method of claim 1 wherein the recombinant virus is delivered to the skin tissue in vitro.

5. The method of claim 4 wherein the skin tissue comprises skin explants and artificial skin tissues.

6. The method of claim 1 wherein the ultrasound is applied prior to administering the recombinant virus.

7. The method of claim 1 wherein the ultrasound is stopped prior to administering the recombinant virus.

8. The method of claim 1 wherein the ultrasound is applied at a frequency between about 20 kHz and about 100 kHz.

9. The method of claim 1 wherein the ultrasound is applied at an intensity between about 1 W/cm² and about 10 W/cm².

10. The method of claim 1 wherein the ultrasound is applied for a duration between about one minute to about 10 minutes.

11. The method of claim 1 wherein the ultrasound is applied at duty cycles in the range of 25%, 50%, 75% or 100%.

12. The method of claim 1 wherein the ultrasound is applied topically or intra-dermally.

13. The method of claim 1 further comprising delivering the recombinant virus to the skin tissue via electroporation, heat, needleless injections, pressure waves generated by laser radiation, fraction laser, or radiofrequency (100 kHz), magnetophoresis by external magnetic field, iontophoresis, chemical peels, abrasion techniques including diamond or sand paper abrasion, tape stripping, and the like.

14. The method of claim 1 wherein the recombinant virus is selected from retrovirus, adenovirus, adeno-associated virus (AAV), vaccinia virus and herpes simplex virus.

15. The method of claim 14 wherein the recombinant AAV includes serotypes 1-9.

16. The method of claim 1 wherein the recombinant virus comprises a heterologous nucleic acid sequence.

17. The method of claim 16 wherein the nucleic acid sequence encodes a gene which is expressible in the skin tissue.

18. The method of claim 17 wherein expression of the gene effects treatment of a skin disease or condition.

19. The method of claim 17 wherein the gene is selected from COL1A1, COL3A1, TIMP1, TIMP2, SMAD2, SMAD3, CTGF, TGF-beta1, KRT6A, NOTCH1(icd), TET2, TET3, Sirt1, Sirt6, HIF-1a, Pten, Pck1, Pparg, and Cisd2, MDH1/2, Aco1/2, IDH1/2/3, Enolase, GOT1/2, MUC1, and MCU.

20. (canceled)

21. The method of claim 1 wherein the skin disease or condition includes Epidermolysis Bullosa, Recessive Dystrophic Epidermolysis Bullosa, Junctional Epidermolysis Bullosa, Epidermolysis Bullosa Simplex, Pachyonychia Congenita, Melanoma, non-melanoma skin cancer, Ichthyosis, Harlequin Ichthyosis, Sjogren-Larsson Syndrome, Xeroderma Pigmentosum, Wound Healing, Netherton Syndrome, age-associated skin pathologies, benign and malignant skin lesions, inflammatory and autoimmune skin disorders.

22. The method of claim 1 wherein the recombinant virus is delivered to keratinocytes, epidermal stem cells, fibroblast cells, mesenchymal stem cells, immune cells, melanocytes, vascular endothelial cells, adipocytes, Merkel cells and peripheral neural cells of the skin tissue.

23. The method of claim 1 wherein the recombinant virus is delivered to skin tissue layers and structures including stratum corneum, epidermis, basement membrane, dermis, hair follicles, blood vessels and sebaceous and eccrine glands.

24. The method of claim 1 wherein multiple recombinant viruses comprising multiple genes are delivered to the skin tissue.

25. The method of claim 2 wherein the subject is human or non-human mammal.

26. (canceled)

27. A recombinant virus comprising a heterologous nucleic acid sequence.

28. The recombinant virus of claim 27 wherein the nucleic acid sequence encodes a gene which is expressible in a skin tissue.

29. The recombinant virus of claim 28 wherein expression of the gene effects treatment of a skin disease or condition.

30. The recombinant virus of claim 28 wherein the gene is selected from COL1A1, COL3A1, TIMP1, TIMP2, SMAD2, SMAD3, CTGF, TGF-beta1, KRT6A, NOTCH1(icd), TET2, TET3, Sirt1, Sirt6, HIF-1a, Pten, Pck1, Pparg, Cisd2, MDH1/2, Aco1/2, IDH1/2/3, Enolase, GOT1/2, MUC1, and MCU.

31. (canceled)

32. The recombinant virus of claim 27 wherein the recombinant virus is selected from retrovirus, adenovirus, adeno-associated virus (AAV), vaccinia virus and herpes simplex virus.

33. The recombinant virus of claim 32 wherein the recombinant AAV includes serotypes 1-9.

34. A method of delivering a polypeptide to a skin tissue comprising
applying ultrasound to the skin tissue, and
administering a nucleic acid sequence encoding the polypeptide to the skin tissue.

35.-58. (canceled)

59. A heterologous nucleic acid sequence encoding a gene which is expressible in a skin tissue.

60. (canceled)

61. The heterologous nucleic acid sequence of claim 59 wherein the gene is selected from COL1A1, COL3A1, TIMP1, TIMP2, SMAD2, SMAD3, CTGF, TGF-beta1, KRT6A, NOTCH1(icd), TET2, TET3, Sirt1, Sirt6, HIF-1a, Pten, Pck1, Pparg, Cisd2, MDH1/2, Aco1/2, IDH1/2/3, Enolase, GOT1/2, MUC1, and MCU.

62. (canceled)

63. The method of claim 17 wherein the gene further comprises sequences of SEQ ID NOS 1-122.
